(19) United States
(12) Patent Application Publication
(10) Pub. No.: US 2013/0105769 A1
(43) Pub. Date: May 2, 2013

(54) CONDENSED-CYCLIC COMPOUND AND ORGANIC LIGHT-EMITTING DEVICE INCLUDING THE SAME

(71) Applicants: Jin-O Lim, Yongin-City (KR); Seok-Hwan Hwang, Yongin-City (KR); Young-Kook Kim, Yongin-City (KR); Hye-Jin Jung, Yongin-City (KR); Sang-Hyun Han, Yongin-City (KR); Soo-Yon Kim, Yongin-City (KR); Hee-Joo Ko, Yongin-City (KR)

(72) Inventors: Jin-O Lim, Yongin-City (KR); Seok-Hwan Hwang, Yongin-City (KR); Young-Kook Kim, Yongin-City (KR); Hye-Jin Jung, Yongin-City (KR); Sang-Hyun Han, Yongin-City (KR); Soo-Yon Kim, Yongin-City (KR); Hee-Joo Ko, Yongin-City (KR)

(73) Assignee: SAMSUNG MOBILE DISPLAY CO., LTD., Yongin-City (KR)

(21) Appl. No.: 13/477,962

(22) Filed: May 22, 2012

(30) Foreign Application Priority Data
Nov. 2, 2011 (KR) 10-2011-0113592

(51) Int. Cl. H01L 51/54; C07D 491/048; C07D 493/04; C07D 487/04

(52) U.S. Cl. 257/40; 548/433; 546/256; 544/212; 546/171; 544/296; 548/430; 548/442; 549/458; 548/305.4; 257/E51.026

(57) ABSTRACT

A condensed-cyclic compound represented by Formula 1, and an organic light-emitting device including the condensed-cyclic compound.

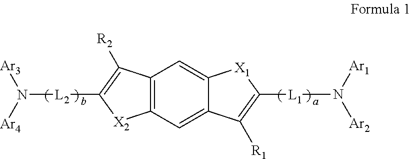

wherein R₁ and R₂, Ar₁ through Ar₄, L₁ and L₂, X₁ and X₂, and a and b are defined as in the specification.

### CLAIM OF PRIORITY

This application makes reference to, incorporates the same herein, and claims all benefits accruing under 35 U.S.C. §119 from an application for CONDENSED-CYCLIC COMPOUND AND ORGANIC LIGHT-EMITTING DEVICE INCLUDING THE SAME earlier filed in the Korean Intellectual Property Office on 2 Nov. 2011 and there duly assigned Serial No. 10-2011-0113592.

### BACKGROUND OF THE INVENTION

1. Field of the Invention

The present invention relates to a condensed-cyclic compound represented by Formula 1 and an organic light-emitting device including the same.

2. Description of the Related Art

Organic light-emitting diodes (OLEDs) are self-emitting devices. The OLEDs have advantages such as a wide viewing angle, excellent contrast, quick response, high brightness, and excellent driving voltage. The OLEDs can provide multicolored images.

A general OLED has a structure including a substrate, and an anode, a hole transport layer (HTL), an emission layer (EML), an electron transport layer (ETL), and a cathode which are sequentially stacked on the substrate. In this regard, the HTL, the EML, and the ETL are organic layers formed of organic compounds.

An operating principle of an OLED having the above-described structure is as follows.

When a voltage is applied between the anode and the cathode, holes injected from the anode move to the EML via the HTL, and electrons injected from the cathode move to the EML via the ETL. The holes and electrons recombine in the EML to generate excitons. When the excitons drop from an excited state to a ground state, light is emitted.

### SUMMARY OF THE INVENTION

The present invention provides a condensed-cyclic compound having a novel structure.

The present invention also provides an organic light-emitting device including the condensed-cyclic compound same.

According to an aspect of the present invention, there is provided a condensed-cyclic compound represented by Formula 1 below:

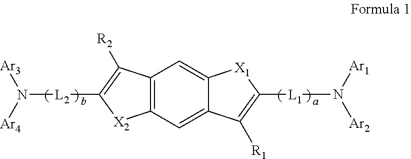

Wherein X₁ may be N(R₁₁) or O; X₂ may be N(R₁₂) or O; R₁, R₂, R₁₁, and R₁₂ may be each independently hydrogen, deuterium, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ aryl group, a substituted or unsubstituted C₅-C₆₀ aryloxy group, a substituted or unsubstituted C₅-C₆₀ arylthio group, a substituted or unsubstituted C₂-C₆₀ heteroaryl group, —N(R₃₁)(R₃₂), or —Si(R₃₃)(R₃₄)(R₃₅); L₁ and L₂ may be each independently a substituted or unsubstituted C₅-C₆₀ arylene group or a substituted or unsubstituted C₂-C₆₀ heteroarylene group; Ar₁ through Ar₄ may be each independently a substituted or unsubstituted C₅-C₆₀ aryl group or a substituted or unsubstituted C₂-C₆₀ heteroaryl group; a and b may be each independently an integer of 1 to 5; and R₃₁ through R₃₅ may be each independently hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ aryl group, a substituted or unsubstituted C₅-C₆₀ aryloxy group, a substituted or unsubstituted C₅-C₆₀ arylthio group, or a substituted or unsubstituted C₂-C₆₀ heteroaryl group.

According to another aspect of the present invention, there is provided an organic light-emitting device comprising a first electrode; a second electrode facing the first electrode; and an organic layer interposed between the first electrode and the second electrode, wherein the organic layer comprises at least one of the condensed-cyclic compounds described above.

### BRIEF DESCRIPTION OF THE DRAWINGS

A more complete appreciation of the invention, and many of the attendant advantages thereof, will be readily apparent as the preent invention becomes better understood by reference to the following detailed description when considered in conjunction with accompanying drawings in which like reference symbols indicate the same or similar components, wherein:

FIG. 1 is a schematic diagram illustrating an organic light-emitting diode (OLED) according to an embodiment of the present invention.

### DETAILED DESCRIPTION OF THE INVENTION

Hereinafter, the present invention will be described more fully with reference to the accompanying drawing, in which exemplary embodiments of the present invention are shown.

As used herein, the term “and/or” includes any and all combinations of one or more of the associated listed items. Expressions such as “at least one of” when preceding a list of elements, modify the entire list of elements and do not modify the individual elements of the list.

According to an embodiment of the present invention, there is provided a condensed-cyclic compound represented by Formula 1 below:

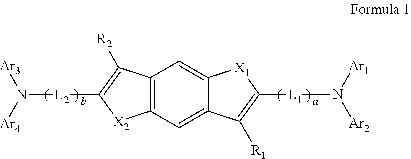

The condensed-cyclic compound may be represented by one of Formulae 1A through 1C below:

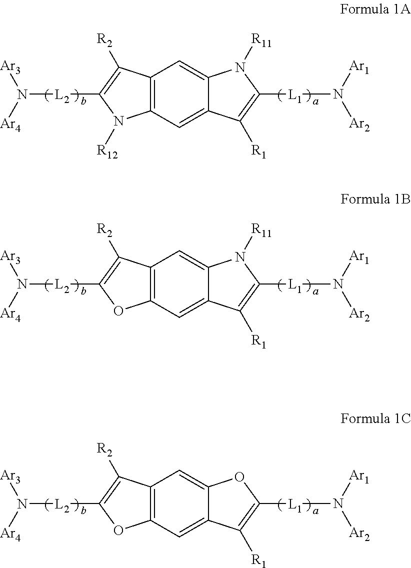

Wherein X₁ may be N(R₁₁) or O, and X₂ may be N(R₁₂) or O.

Wherein R₁, R₂, R₁₁, and R₁, may be each independently hydrogen, deuterium, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ aryl group, a substituted or unsubstituted C₅-C₆₀ aryloxy group, a substituted or unsubstituted C₅-C₆₀ arylthio group, a substituted or unsubstituted C₂-C₆₀ heteroaryl group, —N(R₃₁)(R₃₂), or —Si(R₃₃)(R₃₄)(R₃₅). In this regard, R₃₁ through R₃₅ may be each independently hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ aryl group, a substituted or unsubstituted C₅-C₆₀ aryloxy group, a substituted or unsubstituted C₅-C₆₀ arylthio group, or a substituted or unsubstituted C₂-C₆₀ heteroaryl group.

For example, in Formula 1 above, at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₆₀ cycloalkyl group, the substituted C₅-C₆₀ aryl group, the substituted C₅-C₆₀ aryloxy group, the substituted C₅-C₆₀ arylthio group, the substituted C₂-C₆₀ heteroaryl group, the substituted C₅-C₆₀ arylene group, and the substituted C₂-C₆₀ heteroarylene group may be selected from deuterium; a halogen atom; a hydroxyl group; a nitro group; a cyano group; an amino group; an amidino group; hydrazine; hydrazone; a carboxyl group or a salt thereof; a sulfonic acid group or a salt thereof; a phosphoric acid or a salt thereof; a C₁-C₆₀ alkyl group; a C₂-C₆₀ alkenyl group; a C₂-C₆₀ alkynyl group; a C₆-C₆₀ aryl group; a C₂-C₆₀ heteroaryl group; a C₁-C₆₀ alkyl group, a C₂-C₆₀ alkenyl group, a C₂-C₆₀ alkynyl group, a C₅-C₆₀ aryl group, a C₂-C₆₀ heteroaryl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a phenyl group, and a naphthyl group; —N(Q₁₁)(Q₁₂); and —Si(Q₁₃)(Q₁₄)(Q₁₅); and wherein Q₁₁ through Q₁₅ may be each independently hydrogen; a C₁-C₆₀ alkyl group; a C₅-C₆₀ aryl group; a C₅-C₆₀ aryl group that is substituted with deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₆₀ alkyl group, or a C₁-C₆₀ alkoxy group; a C₂-C₆₀ heteroaryl group; or a C₂-C₆₀ heteroaryl group that is substituted with deuterium, a halogen atom, a hydroxyl group, a cyano group, a nitro group, an amino group, an amidino group; hydrazine, hydrazone, a carboxyl group or a salt thereof, a sulfonic acid group or a salt thereof, a phosphoric acid or a salt thereof, a C₁-C₆₀ alkyl group, or a C₁-C₆₀ alkoxy group.

For example, in Formula 1 above, R₁, R₂, R₁₁, and R₁₂ may be each independently, but are not limited to, hydrogen, deuterium, a substituted or unsubstituted C₁-C₂₀ alkyl group, a substituted or unsubstituted C₁-C₂₀ alkoxy group, a substituted or unsubstituted phenyl group; a substituted or unsubstituted pentalenyl group, a substituted or unsubstituted indenyl group, a substituted or unsubstituted naphtyl group, a substituted or unsubstituted azulenyl group, a substituted or unsubstituted heptalenyl group, a substituted or unsubstituted indacenyl group, a substituted or unsubstituted acenaphtyl group, a substituted or unsubstituted fluorenyl group, a substituted or unsubstituted spiro-fluorenyl group, a substituted or unsubstituted phenyllenyl group, a substituted or unsubstituted phenanthrenyl group, a substituted or unsubstituted anthryl group, a substituted or unsubstituted fluoranthenyl group, a substituted or unsubstituted triphenylenyl group, a substituted or unsubstituted pyrenyl group, a substituted or unsubstituted chrysenyl group, a substituted or unsubstituted naphthacenyl group. a substituted or unsubstituted picenyl group, a substituted or unsubstituted perylenyl group, a substituted or unsubstituted pentaphenyl group, a substituted or unsubstituted hexacenyl group, a substituted or unsubstituted pyrrolyl group, a substituted or unsubstituted imidazolyl group, a substituted or unsubstituted pyrazolyl group, a substituted or unsubstituted pyridinyl group, a substituted or unsubstituted pyrazinyl group, a substituted or unsubstituted pyrimidinyl group, a substituted or unsubstituted pyridazinyl group, a substituted or unsubstituted isoindolyl group, a substituted or unsubstituted indolyl group, a substituted or unsubstituted indazolyl group, a substituted or unsubstituted purinyl group, a substituted or unsubstituted quinolinyl group, a substituted or unsubstituted benzoquinolinyl group, a substituted or unsubstituted phthalazinyl group, a substituted or unsubstituted naphthyridinyl group, a substituted or unsubstituted quinoxalinyl group, a substituted or unsubstituted quinazolinyl group, a substituted or unsubstituted cinnolinyl group, a substituted or unsubstituted carbazolyl group, a substituted or unsubstituted phenanthridinyl group, a substituted or unsubstituted acridinyl group, a substituted or unsubstituted phenanthrolinyl group, a substituted or unsubstituted phenazinyl group, a substituted or unsubstituted benzooxazolyl, a substituted or unsubstituted benzoimidazolyl group, a substituted or unsubstituted furanyl group, a substituted or unsubstituted benzofuranyl group, a substituted or unsubstituted thiophenyl group, a substituted or unsubstituted benzothiophenyl group, a substituted or unsubstituted thiazolyl group, a substituted or unsubstituted isothiazolyl group, a substituted or unsubstituted benzothiazolyl group, a substituted or unsubstituted isoxazolyl group, a substituted or unsubstituted oxazolyl group, a substituted or unsubstituted triazolyl group, a substituted or unsubstituted tetrazolyl group, a substituted or unsubstituted oxadiazolyl group, a substituted or unsubstituted triazinyl group, a substituted or unsubstituted benzooxazolyl group, a substituted or unsubstituted dibenzopuranyl group, a substituted or unsubstituted dibenzothiophenyl group, or a substituted or unsubstituted benzocarbazolyl group.

For example, in Formula 1 above, R₁₁ and R₁₂ may be each independently, but are not limited to, a substituted or unsubstituted C₁-C₂₀ alkyl group or one of Formulae 3-1 through 3-27 below:

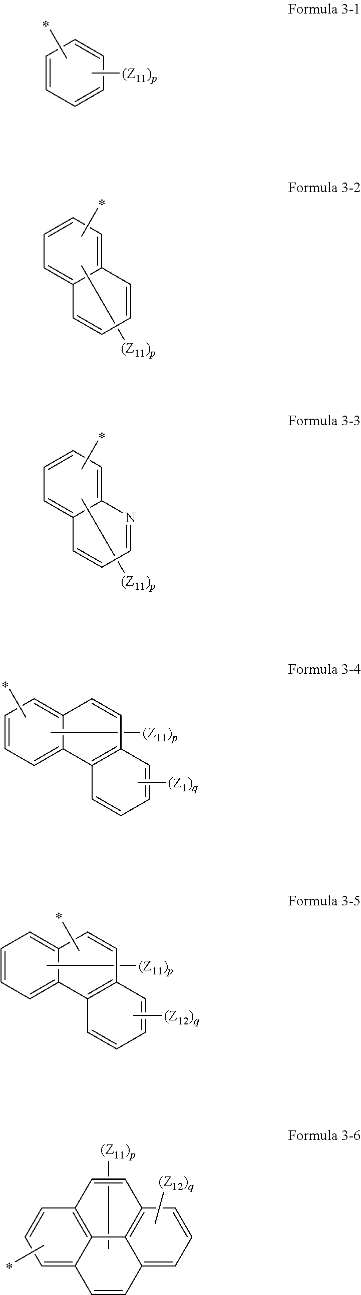

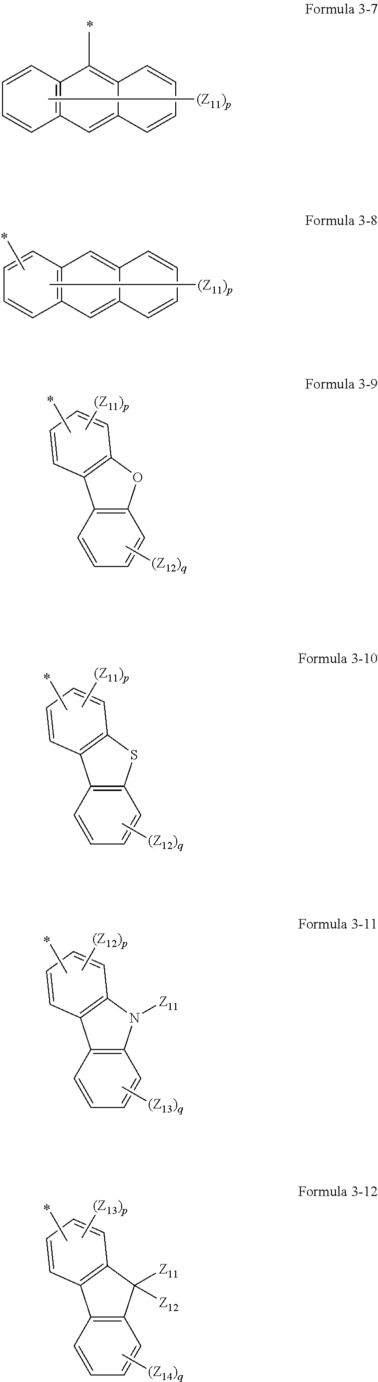

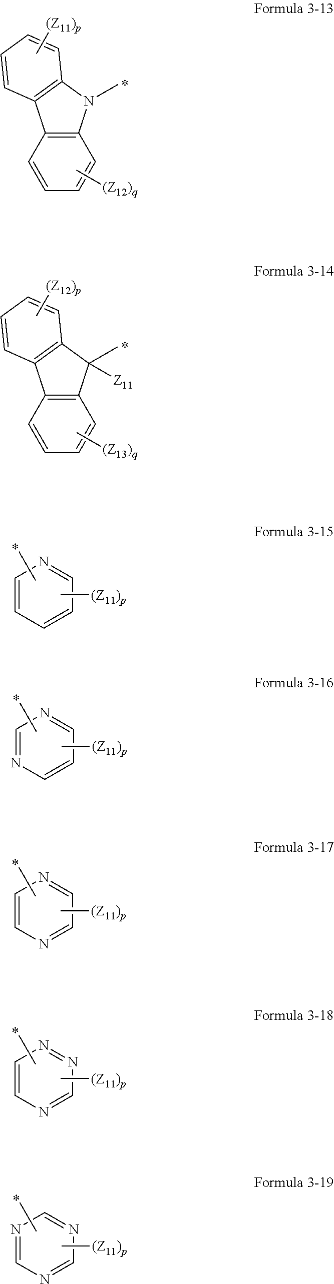

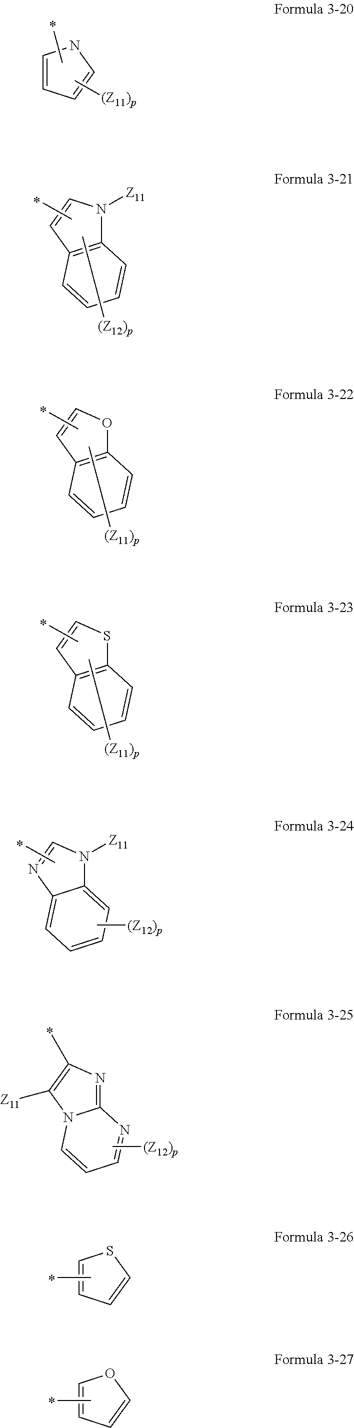

Wherein Z₁₁ through Z₁₄ may be each independently one of hydrogen; deuterium; a halogen atom; a hydroxyl group; a cyano group; a nitro group; an amino group; an amidino group; hydrazine; hydrazone; a carboxyl group or a salt thereof; a sulfonic acid group or a salt thereof; a phosphoric acid or a salt thereof; a C₁-C₁₀ alkyl group; a C₁-C₁₀ alkoxy group; a C₁-C₁₀ alkyl group and a C₁-C₁₀ alkoxy group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof; a phenyl group; a naphthyl group; a fluorenyl group; a phenanthrenyl group; an anthryl group; a pyrenyl group; a chrysenyl group; a phenyl group, a naphthyl group, a fluorenyl group, a phenanthrenyl group, an anthryl group, a pyrenyl group, and a chrysenyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, and a C₁-C₁₀ alkoxy group; an indolyl group; a benzoimidazolyl group; a carbazolyl group; an imidazolyl group; an imidazolinyl group; an imidazopyridinyl group; an imidazopyrimidinyl group; a pyridinyl group; a pyrimidinyl group; a triazinyl group; a quinolinyl group; an indolyl group, a benzoimidazolyl group, a carbazolyl group, an imidazolyl group, an imidazolinyl group, an imidazopyridinyl group, an imidazopyrimidinyl group, a pyridinyl group, a pyrimidinyl group, a triazinyl group, and a quinolinyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a phenyl group, and a naphthyl group; and —N(Q₁₁)(Q₁₂), p may be an integer of 1 to 9, and q may be an integer of 1 to 4.

Wherein Q₁₁ and Q₁₂ may be each independently one of a phenyl group; a naphthyl group; a fluorenyl group; a phenanthrenyl group; an anthryl group; a pyrenyl group; a chrysenyl group; and a phenyl group, a naphthyl group, a fluorenyl group, a phenanthrenyl group, an anthryl group, a pyrenyl group, and a chrysenyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, and a C₁-C₁₀ alkoxy group.

In Formula 1 above, R₁₁ and R₁₂ may be each independently one of a methyl group; an ethyl group; a propyl group; a butyl group; a pentyl group; a hexyl group; a heptyl group; a methyl group, an ethyl group, a propyl group, a butyl group, a pentyl group, a hexyl group, and a heptyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof; and Formulae 4-1 through 4-41:

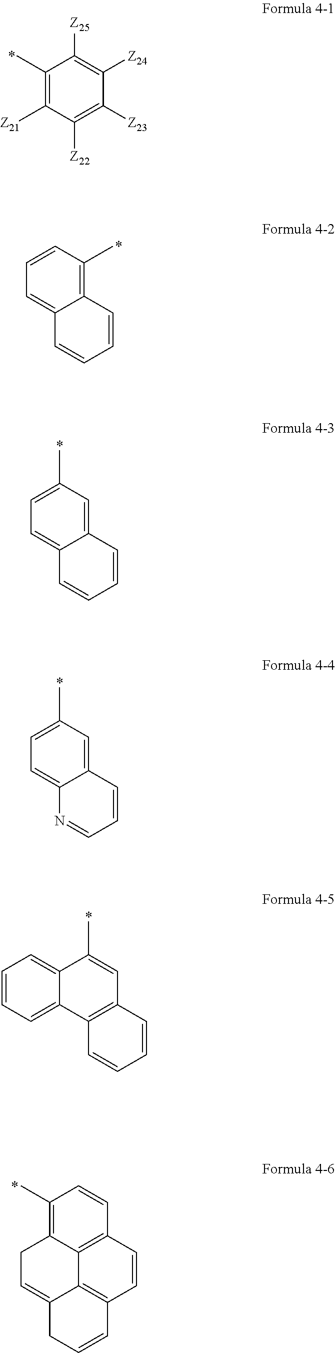

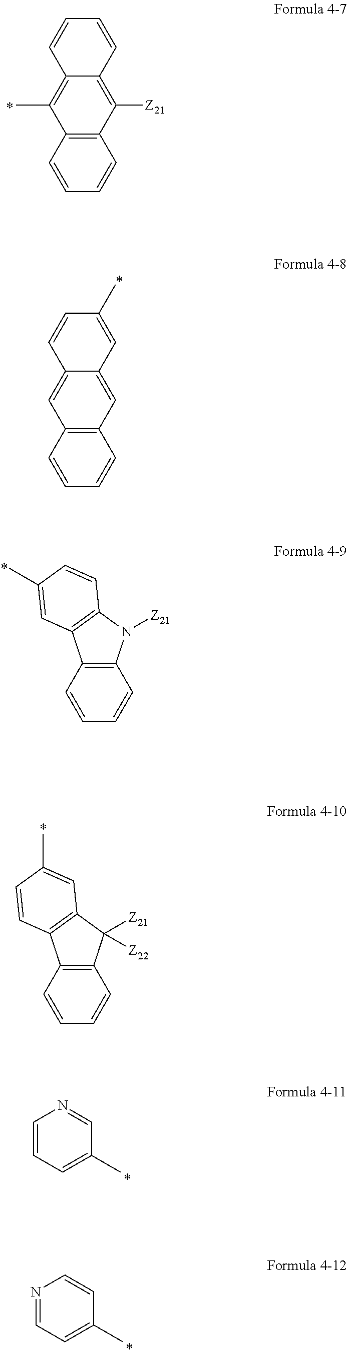

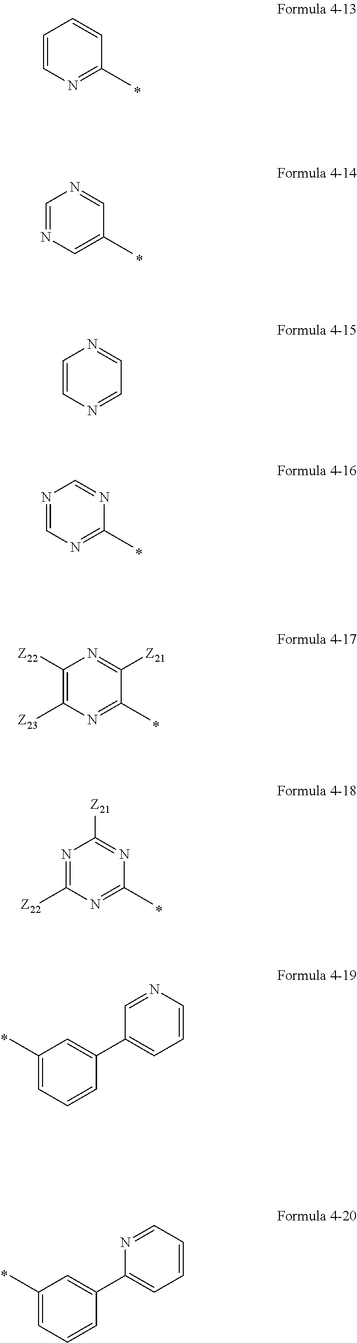

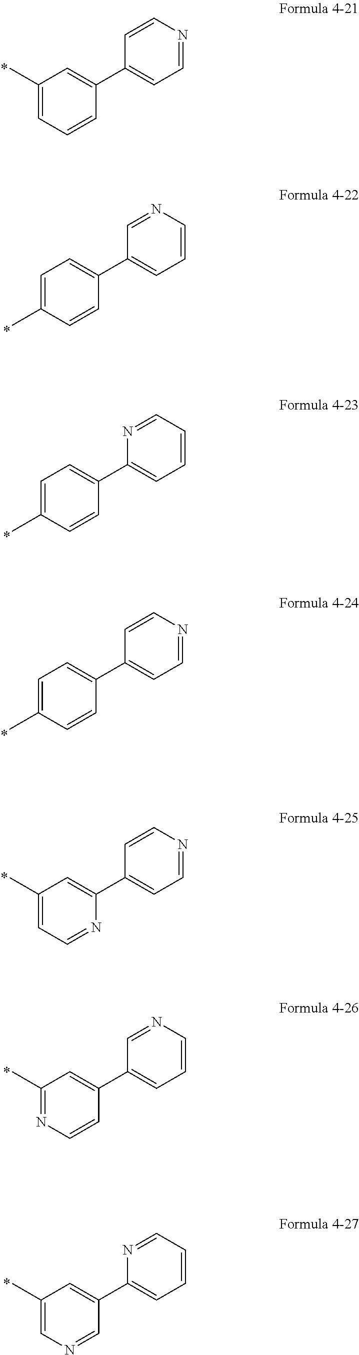

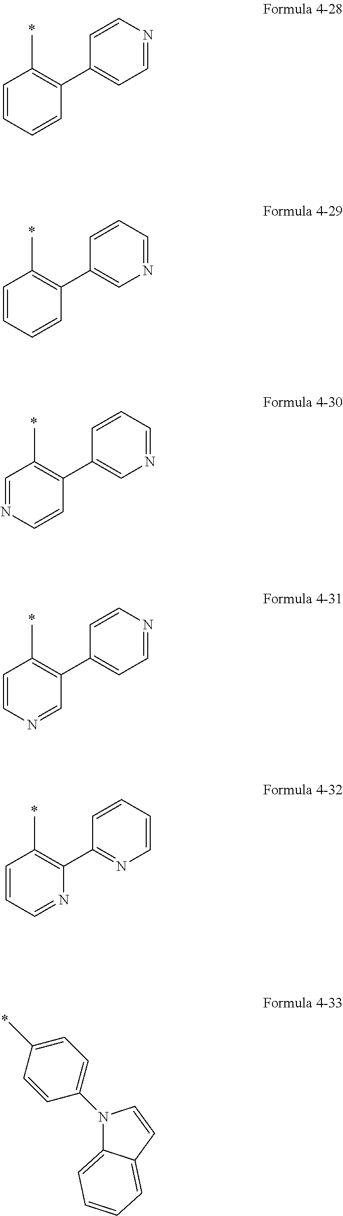 [SMILES: Cc1ccccc1-c1ccncc1 ; Cc1ccccc1-c1cccnc1 ; Cc1cnccc1-c1cccnc1 ; Cc1ccncc1-c1ccncc1 ; Cc1cccnc1-c1ccccn1 ; Cc1ccc(-n2ccc3ccccc32)cc1]

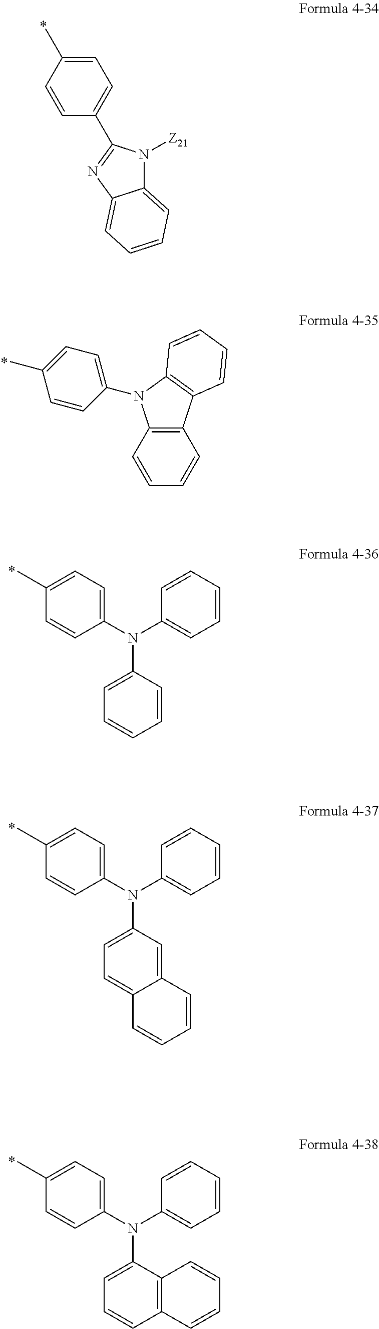

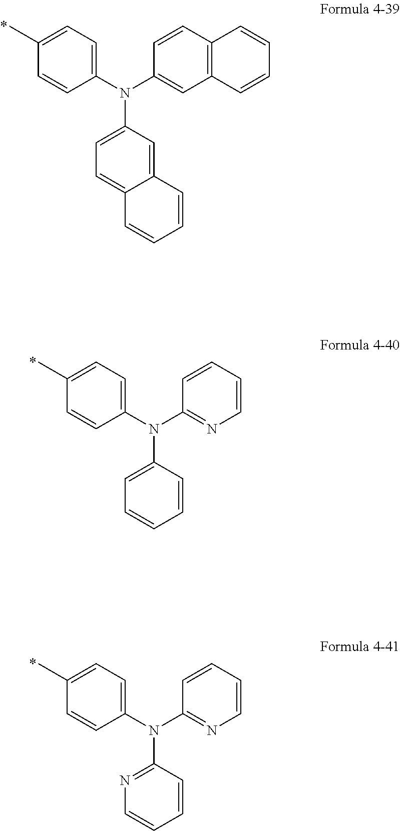 [SMILES: Cc1ccc(N(c2ccc3ccccc3c2)c2ccc3ccccc3c2)cc1 ; Cc1ccc(N(c2ccccc2)c2ccccn2)cc1 ; Cc1ccc(N(c2ccccn2)c2ccccn2)cc1]

Wherein Z₂₁ through Z₂₅ may be each independently hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a phenyl group, a naphthyl group, a fluorenyl group, a phenanthrenyl group, an anthryl group, a pyrenyl group, a chrysenyl group, an indolyl group, a benzoimidazolyl group, a carbazolyl group, an imidazolyl group, an imidazolinyl group, an imidazopyridinyl group, an imidazopyrimidinyl group, a pyridinyl group, a pyrimidinyl group, a triazinyl group, or a quinolinyl group.

For example, in Formula 1 above, R₁₁ and R₁₂ may be each independently, but are not limited to, one of a methyl group; an ethyl group; a propyl group; a butyl group; a pentyl group; a hexyl group; a heptyl group; a methyl group, an ethyl group, a propyl group, a butyl group, a pentyl group, a hexyl group, and a heptyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof; and Formulae 4-1, 4-2, 4-3,4-5, 4-6, 4-9, 4-10, 4-11, 4-18, 4-19, 4-33, 4-34, and 4-36.

In Formula 1 above, R₁ and R₂ may be each independently, but are not limited to, hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a phenyl group, a naphthyl group, a fluorenyl group, a phenanthrenyl group, an anthryl group, or a pyrenyl group. For example, in Formula 1 above, R₁ and R₂ may be each independently hydrogen or a phenyl group, but are not limited thereto.

In Formula 1 above, L₁ and L₂ may be each independently a substituted or unsubstituted C₅-C₆₀ arylene group or a substituted or unsubstituted C₂-C₆₀ heteroarylene group.

In Formula 1 above, L₁ and L₂ may be each independently a substituted or unsubstituted phenylenyl group, a substituted or unsubstituted pentalenylene group, a substituted or unsubstituted indenylene group, a substituted or unsubstituted naphthylene group, a substituted or unsubstituted azulenylene group, a substituted or unsubstituted heptalenylene group, a substituted or unsubstituted indacenylene group, a substituted or unsubstituted acenaphthylene group, a substituted or unsubstituted fluorenylene group, a substituted or unsubstituted phenalenylene group, a substituted or unsubstituted phenanthrenylene group, a substituted or unsubstituted anthrylene group, a substituted or unsubstituted fluoranthenylene group, a substituted or unsubstituted triphenylenylene group, a substituted or unsubstituted pyrenylene group, a substituted or unsubstituted chrysenylene group, a substituted or unsubstituted naphthacenylene group, a substituted or unsubstituted picenylene group, a substituted or unsubstituted perylenylene group, a substituted or unsubstituted pentaphenylene group, a substituted or unsubstituted hexacenylene group, a substituted or unsubstituted pyrrolylene group, a substituted or unsubstituted pyrazolylene group, a substituted or unsubstituted imidazolylene group, a substituted or unsubstituted imidazolinylene group, a substituted or unsubstituted imidazopyridinylene group, a substituted or unsubstituted imidazopyrimidinylene group, a substituted or unsubstituted pyridinylene group, a substituted or unsubstituted pyrazinylene group, a substituted or unsubstituted pyrimidinylene group, a substituted or unsubstituted indolylene group, a substituted or unsubstituted purinylene group, a substituted or unsubstituted quinolinylene group, a substituted or unsubstituted phthalazinylene group, a substituted or unsubstituted indolizinylene group, a substituted orunsubstituted naphthyridinylene group, a substituted or unsubstituted quinazolinylene group, a substituted or unsubstituted cinnolinylene group, a substituted or unsubstituted indazolylene group, a substituted or unsubstituted carbazolylene group, a substituted or unsubstituted phenazinylene group, a substituted or unsubstituted phenanthridinylene group, a substituted or unsubstituted pyranylene group, a substituted or unsubstituted chromenylene group, a substituted or unsubstituted furanylene group, a substituted or unsubstituted benzofuranylene group, a substituted or unsubstituted thiophenylene group, a substituted or unsubstituted benzothiophenylene group, a substituted or unsubstituted isothiazolylene group, a substituted or unsubstituted benzoimidazolylene group, a substituted or unsubstituted isoxazolylene group, a substituted or unsubstituted dibenzothiophenylene group, a substituted or unsubstituted dibenzofuranylene group, a substituted or unsubstituted triazinylene group, or a substituted or unsubstituted oxadiazolylene group.

In Formula 1 above, L₁ and L₂ may be each independently selected from Formulae 2-1 through 2-8 below:

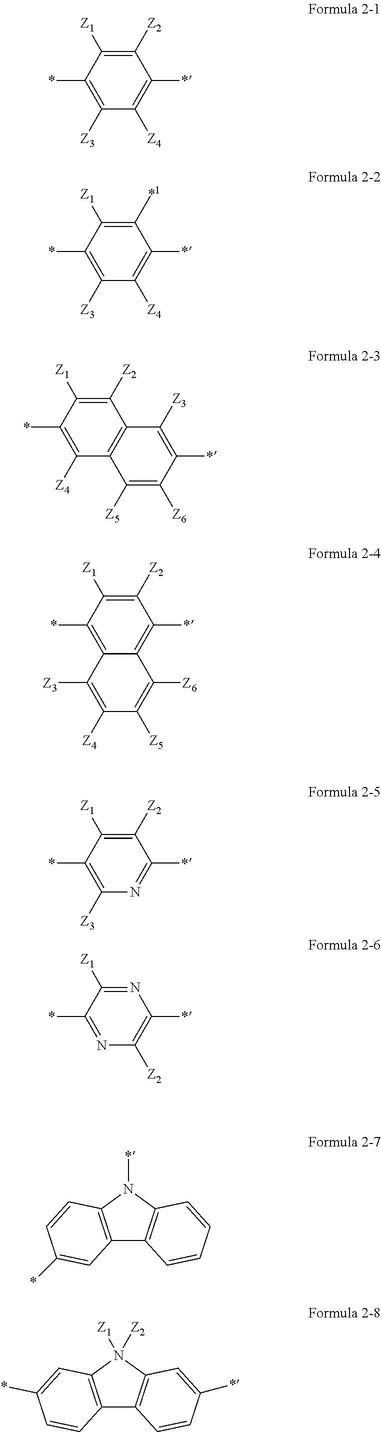

Wherein Z₁ through Z₆ may be each independently one of hydrogen; deuterium; a halogen atom; a hydroxyl group; a cyano group; a nitro group; an amino group; an amidino group; hydrazine; hydrazone; a carboxyl group or a salt thereof; a sulfonic acid group or a salt thereof; a phosphoric acid or a salt thereof; a C₁-C₁₀ alkyl group; a C₁-C₁₀ alkoxy group; a C₁-C₁₀ alkyl group and a C₁-C₁₀ alkoxy group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof; a phenyl group; a naphthyl group; a fluorenyl group; a phenanthrenyl group; an anthryl group; a pyrenyl group; a chrysenyl group; a phenyl group, a naphthyl group, a fluorenyl group, a phenanthrenyl group, an anthryl group, a pyrenyl group, and a chrysenyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, and a C₁-C₁₀ alkoxy group; an indolyl group; a benzoimidazolyl group; a carbazolyl group; an imidazolyl group; an imidazolinyl group; an imidazopyridinyl group; an imidazopyrimidinyl group; a pyridinyl group; a pyrimidinyl group; a triazinyl group; a quinolinyl group; and an indolyl group, a benzoimidazolyl group, a carbazolyl group, an imidazolyl group, an imidazolinyl group, an imidazopyridinyl group, an imidazopyrimidinyl group, a pyridinyl group, a pyrimidinyl group, a triazinyl group, and a quinolinyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a phenyl group, and a naphthyl group.

For example, Z₁ through Z₆ may be each independently, but are not limited to, hydrogen; deuterium; a halogen atom; a hydroxyl group; a cyano group; a nitro group; an amino group; an amidino group; hydrazine; hydrazone; a carboxyl group or a salt thereof; a sulfonic acid group or a salt thereof; a phosphoric acid or a salt thereof; a C₁-C₁₀ alkyl group; a C₁-C₁₀ alkoxy group; a phenyl group; or a naphthyl group.

For example, in Formula 1 above, L₁ and L₂ may be each independently, but are not limited to, a phenylene group, a naphthylene group, a pyridinylene group, a pyrazinylene group, a carbazolylene group, or a fluorenylene group.

In Formula 1 above, L₁ and L₂ may be identical to each other, but are not limited thereto.

In Formula 1 above, Ar₁ through Ar₄ may be each independently a substituted or unsubstituted C₅-C₆₀ aryl group or a substituted or unsubstituted C₂-C₆₀ heteroaryl group.

For example, Ar₁ through Ar₄ may be each independently, but are not limited to, a substituted or unsubstituted phenyl group, a substituted or unsubstituted pentalenyl group, a substituted or unsubstituted indenyl group, a substituted or unsubstituted naphtyl group, a substituted or unsubstituted azulenyl group, a substituted or unsubstituted heptalenyl group, a substituted or unsubstituted indacenyl group, a substituted or unsubstituted acenaphtyl group, a substituted or unsubstituted fluorenyl group, a substituted or unsubstituted spiro-fluorenyl group, a substituted or unsubstituted phenalenyl group, a substituted or unsubstituted phenanthrenyl group, a substituted or unsubstituted anthryl group, a substituted or unsubstituted fluoranthenyl group, a substituted or unsubstituted triphenylenyl group, a substituted or unsubstituted pyrenyl group, a substituted or unsubstituted chrysenyl group, a substituted or unsubstituted naphthacenyl group, a substituted or unsubstituted picenyl group, a substituted or unsubstituted perylenyl group, a substituted or unsubstituted pentaphenyl group, a substituted or unsubstituted hexacenyl group, a substituted or unsubstituted pyrrolyl group, a substituted or unsubstituted imidazolyl group, a substituted or unsubstituted pyrazolyl group, a substituted or unsubstituted pyridinyl group, a substituted or unsubstituted pyrazinyl group, a substituted or unsubstituted pyrimidinyl group, a substituted or unsubstituted pyridazinyl group, a substituted or unsubstituted isoindolyl group, a substituted or unsubstituted indolyl group, a substituted or unsubstituted indazolyl group, a substituted or unsubstituted purinyl group, a substituted or unsubstituted quinolinyl group, a substituted or unsubstituted benzoquinolinyl group, a substituted or unsubstituted phthalazinyl group, a substituted or unsubstituted naphthyridinyl group, a substituted or unsubstituted quinoxalinyl group, a substituted or unsubstituted quinazolinyl group, a substituted or unsubstituted cinnolinyl group, a substituted or unsubstituted carbazolyl group, a substituted or unsubstituted phenanthridinyl group, a substituted or unsubstituted acridinyl group, a substituted or unsubstituted phenanthrolinyl group, a substituted or unsubstituted phenazinyl group, a substituted or unsubstituted benzooxazolyl group, a substituted or unsubstituted benzoimidazolyl group, a substituted or unsubstituted furanyl group, a substituted or unsubstituted benzofuranyl group, a substituted or unsubstituted thiophenyl group, a substituted or unsubstituted benzothiophenyl group, a substituted or unsubstituted thiazolyl, a substituted or unsubstituted isothiazolyl group, a substituted or unsubstituted benzothiazolyl group, a substituted or unsubstituted isoxazolyl group, a substituted or unsubstituted oxazolyl group, a substituted or unsubstituted triazolyl group, a substituted or unsubstituted tetrazolyl group, a substituted or unsubstituted oxadiazolyl group, a substituted or unsubstituted triazinyl group, a substituted or unsubstituted benzooxazolyl group, a substituted or unsubstituted dibenzopuranyl group, a substituted or unsubstituted dibenzothiophenyl group, or a substituted or unsubstituted benzocarbazolyl group.

In Formula 1 above, for example, Ar₁ through Ar₄ may be each independently one of Formulae 3-1 through 3-27.

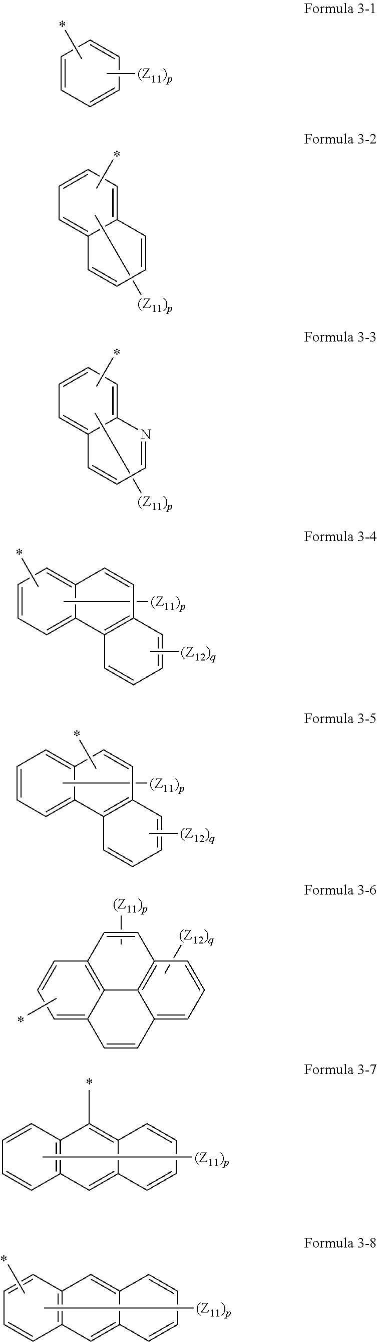

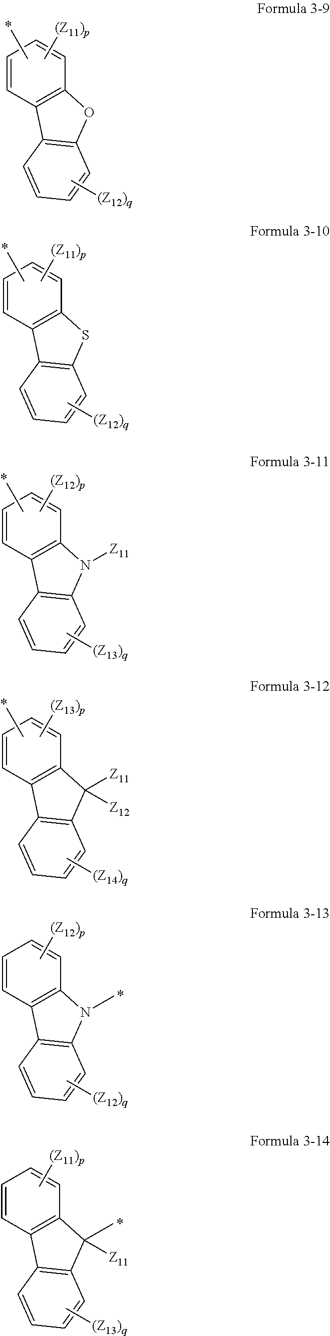

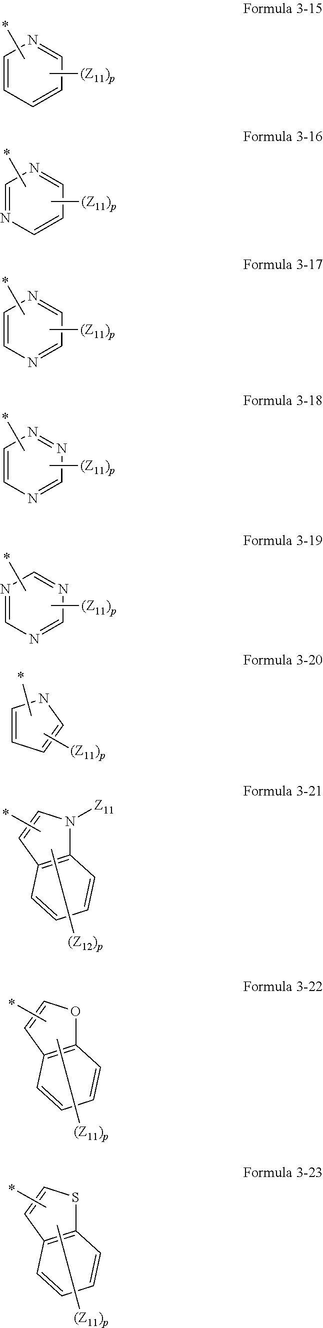

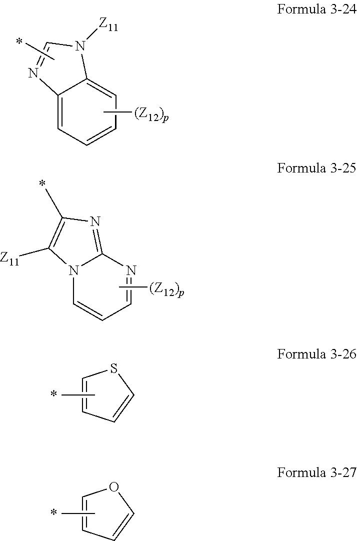

Wherein Z₁₁ through Z₁₄ may be each independently one of hydrogen; deuterium; a halogen atom; a hydroxyl group; a cyano group; a nitro group; an amino group; an amidino group; hydrazine; hydrazone; a carboxyl group or a salt thereof; a sulfonic acid group or a salt thereof; a phosphoric acid or a salt thereof; a C₁-C₁₀ alkyl group; a C₁-C₁₀ alkoxy group; a C₁-C₁₀ alkyl group and a C₁-C₁₀ alkoxy group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof; a phenyl group; a naphthyl group; a fluorenyl group; a phenanthrenyl group; an anthryl group; a pyrenyl group; a chrysenyl group; a phenyl group, a naphthyl group, a fluorenyl group, a phenanthrenyl group, an anthryl group, a pyrenyl group, and a chrysenyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, and a C₁-C₁₀ alkoxy group; an indolyl group; a benzoimidazolyl group; a carbazolyl group; an imidazolyl group; an imidazolinyl group; an imidazopyridinyl group; an imidazopyrimidinyl group; a pyridinyl group; a pyrimidinyl group; a triazinyl group; a quinolinyl group; and an indolyl group, a benzoimidazolyl group, a carbazolyl group, an imidazolyl group, an imidazolinyl group, an imidazopyridinyl group, an imidazopyrimidinyl group, a pyridinyl group, a pyrimidinyl group, a triazinyl group, and a quinolinyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a phenyl group, and a naphthyl group; p may be an integer of 1 to 9; and q may be an integer of 1 to 4.

In another embodiment, Ar₁ through Ar₄ may be each independently, but are not limited to, one of Formulae 4-1 through 4-35 above.

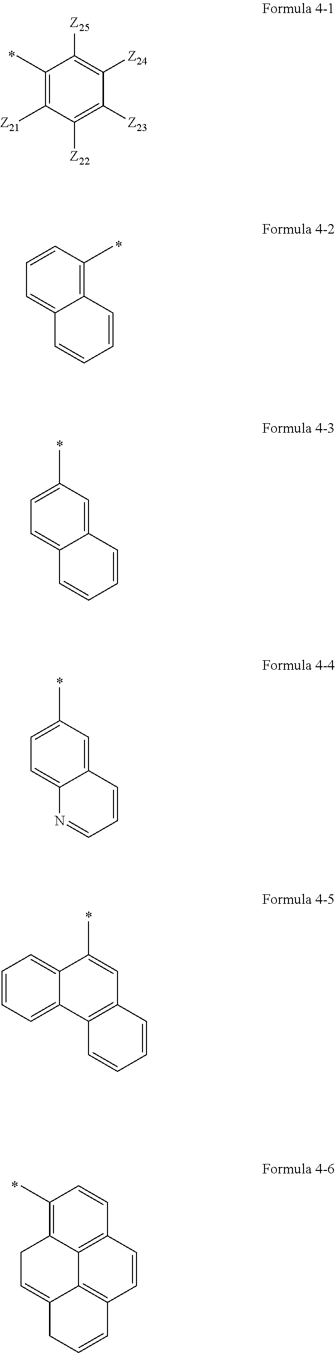

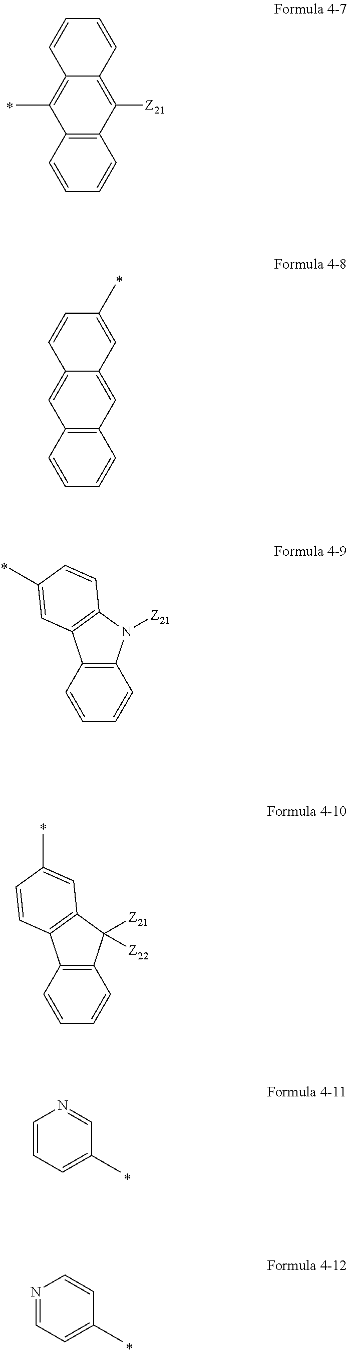

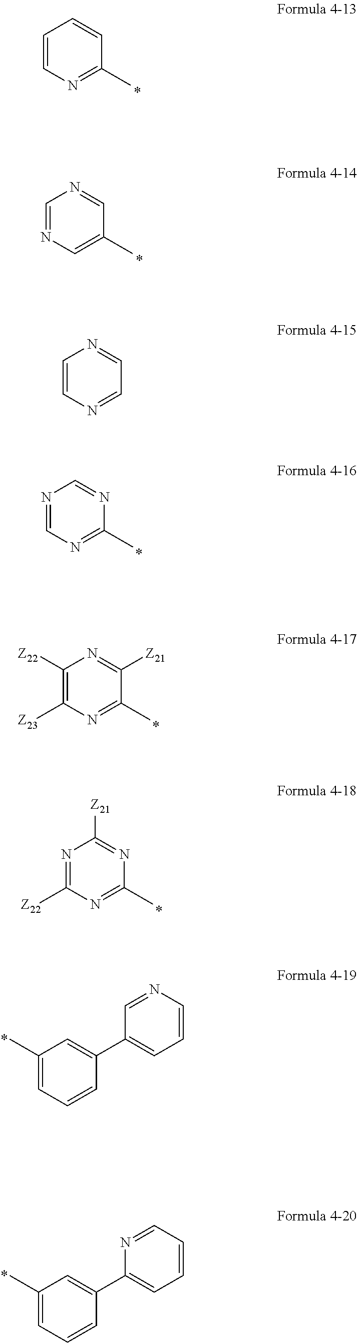

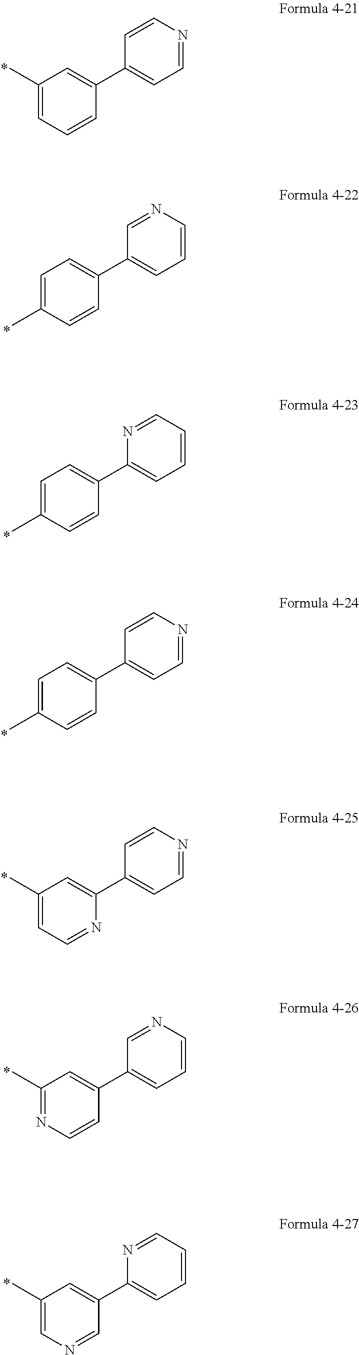

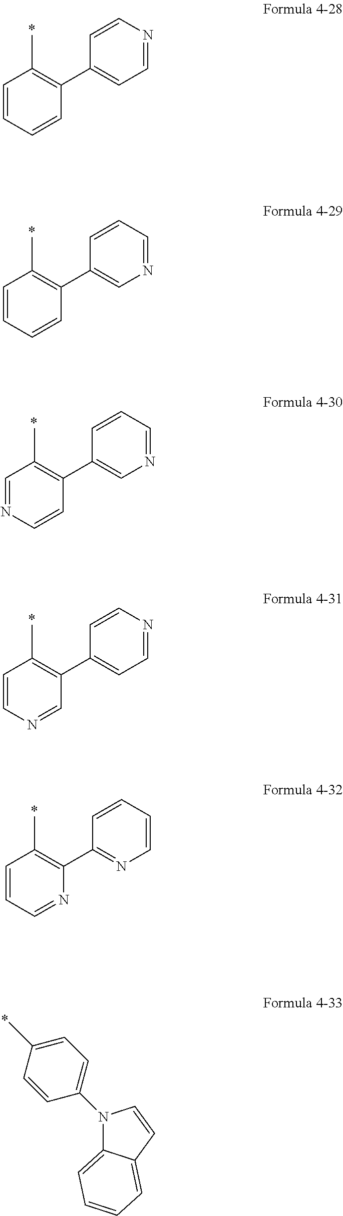 [SMILES: Cc1ccccc1-c1ccncc1 ; Cc1ccccc1-c1cccnc1 ; Cc1cnccc1-c1cccnc1 ; Cc1ccncc1-c1ccncc1 ; Cc1cccnc1-c1ccccn1 ; Cc1ccc(-n2ccc3ccccc32)cc1]

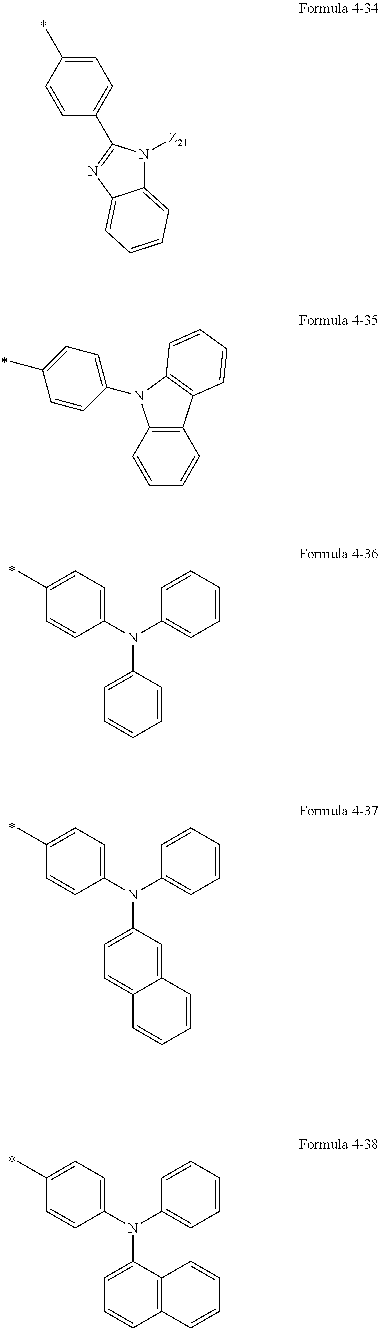

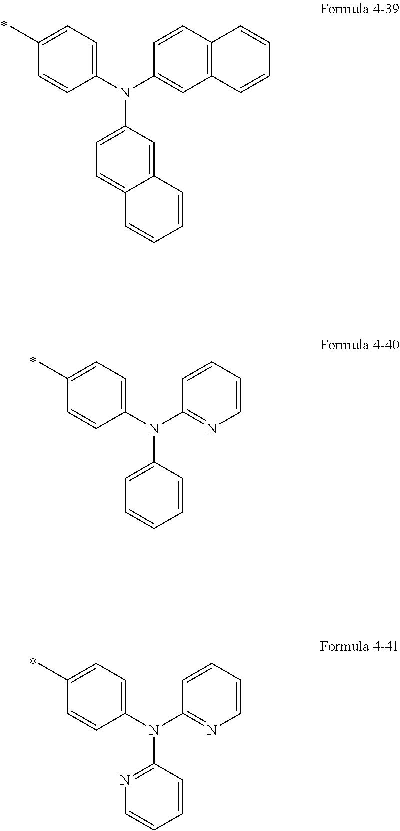 [SMILES: Cc1ccc(N(c2ccc3ccccc3c2)c2ccc3ccccc3c2)cc1 ; Cc1ccc(N(c2ccccc2)c2ccccn2)cc1 ; Cc1ccc(N(c2ccccn2)c2ccccn2)cc1]

Wherein Z₂₁ through Z₂₅ are each independently hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a phenyl group, a naphthyl group, a fluorenyl group, a phenanthrenyl group, an anthryl group, a pyrenyl group, a chrysenyl group, an indolyl group, a benzoimidazolyl group, a carbazolyl group, an imidazolyl group, an imidazolinyl group, an imidazopyridinyl group, an imidazopyrimidinyl group, a pyridinyl group, a pyrimidinyl group, a triazinyl group, or a quinolinyl group.

In Formula 1 above, a and b may be each independently an integer of 1 to 5. In some embodiments, a and b may be each independently an integer of 1 or 2. If a is 2 or more, at least two L₁ groups may be identical to or different from each other, and if b is 2 or more, at least two L₂ groups may be identical to or different from each other.

The condensed-cyclic compound may be represented by Formula 1A above wherein R₁₁ and R₁₂ may be each independently one of a methyl group; an ethyl group; a propyl group; a butyl group; a pentyl group; a hexyl group; a heptyl group; a methyl group, an ethyl group, a propyl group, a butyl group, a pentyl group, a hexyl group, and a heptyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof; and Formulae 4-1 through 4-3, 4-9 through 4-11, 4-18, 4-33, and 4-34; R₁ and R₂ may be each independently hydrogen or a phenyl group; L₁ and L₂ may be each independently a phenylene group, a naphthylene group, a pyridinylene group, a pyrazinylene group, a carbazolylene group, or a fluorenylene group; a and b may be each independently 1 or 2; and Ar₁ through Ar₄ may be each independently one of Formulae 4-1 through 4-4, 4-9, 4-10, and 4-12 through 4-14, but is not limited thereto.

Also, the condensed-cyclic compound may be represented by Formula 1B above wherein R₁₁ may be Formulae 4-1, 4-3, 4-5, 4-6, 4-9, 4-10, 4-19, or 4-36; R₁ and R₂ may be each independently hydrogen or a phenyl group; L₁ and L₂ may be each independently a phenylene group, a naphthylene group, a pyridinylene group, a pyrazinylene group, a carbazolylene group, or a fluorenylene group; a and b may be each independently 1 or 2; and Ar₁ through Ar₄ may be each independently one of Formulae 4-1, 4-2, and 4-9 through 4-13, but is not limited thereto.

Also, the condensed-cyclic compound may be represented by Formula 1C above wherein R₁ and R₂, may be each independently hydrogen or a phenyl group; L₁ and L₂ may be each independently a phenylene group, a naphthylene group, a pyridinylene group, a pyrazinylene group, a carbazolylene group, or a fluorenylene group; a and b may be each independently 1 or 2; and Ar₁ through Ar₄ may be each independently one of Formulae 4-1, 4-2, and 4-9 through 4-13, but is not limited thereto.

The condensed-cyclic compound of Formula 1 may be, for example, any one of Compounds 1 through 68 below, but is not limited thereto:

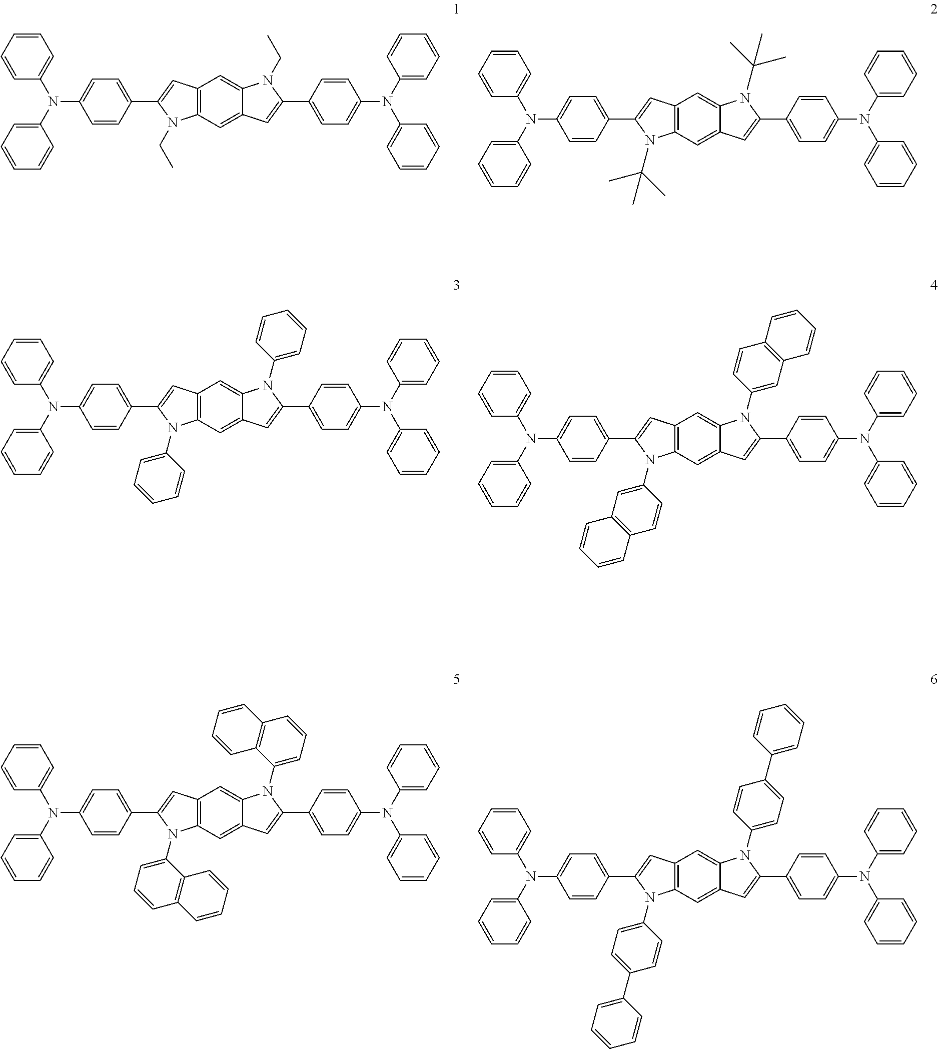

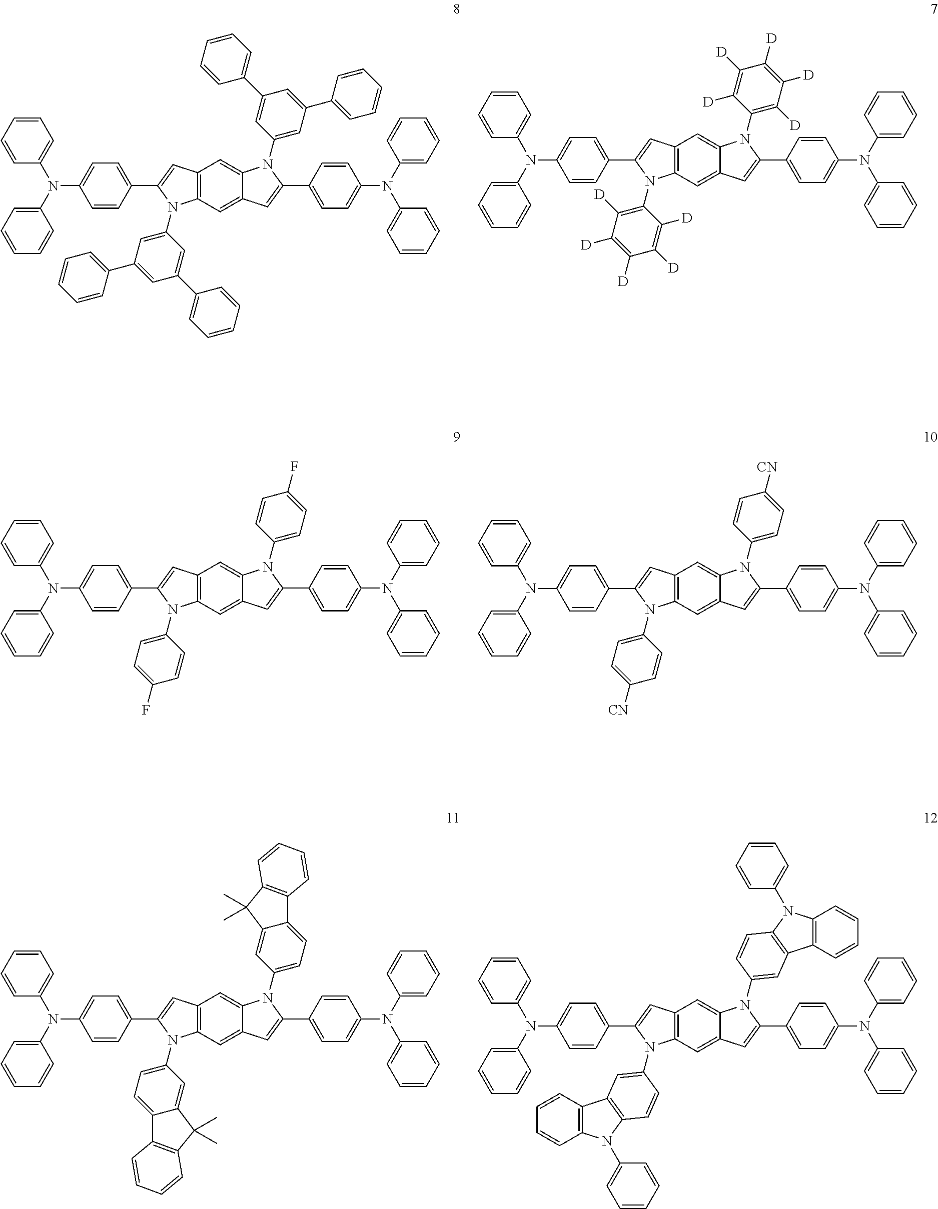

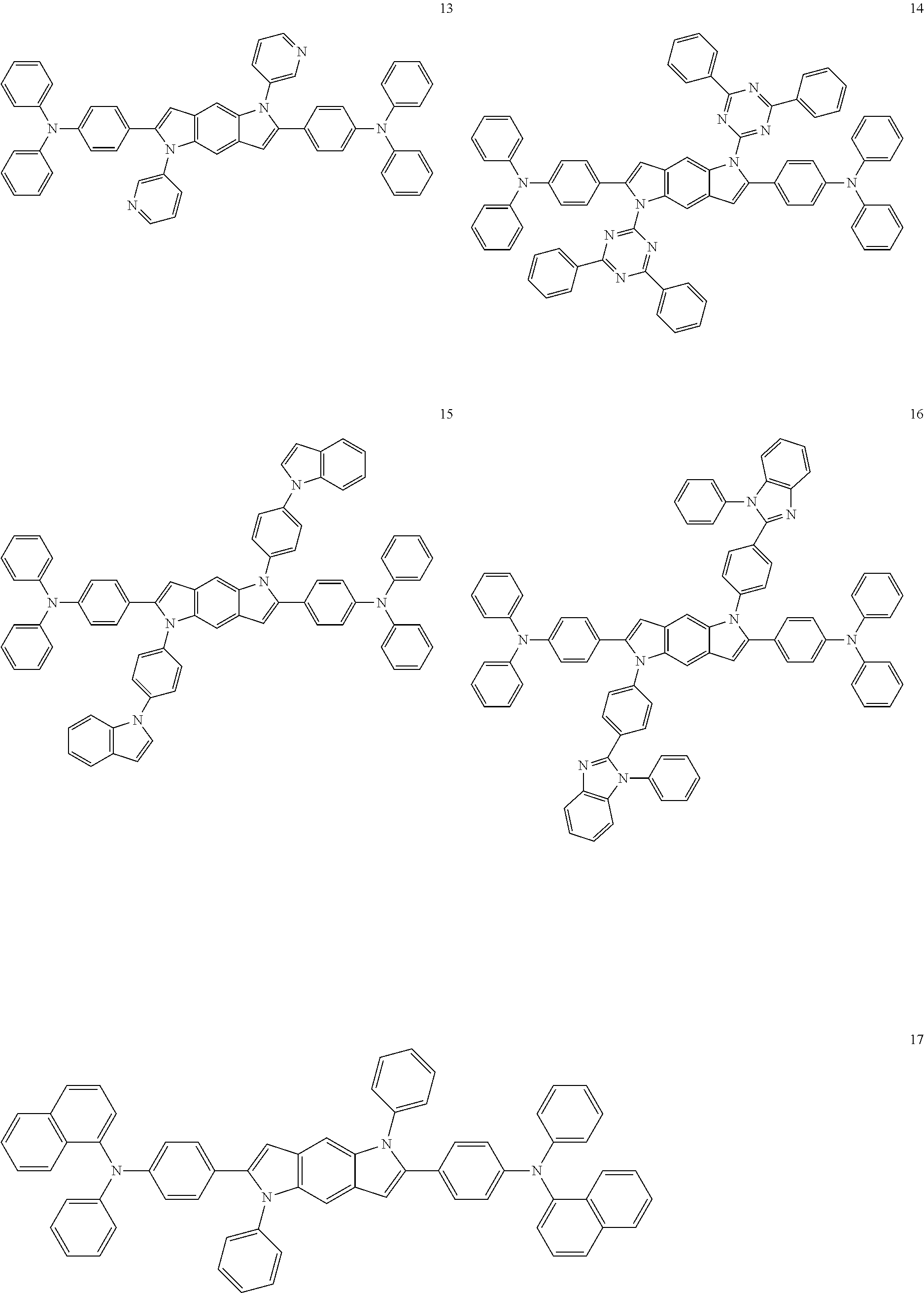

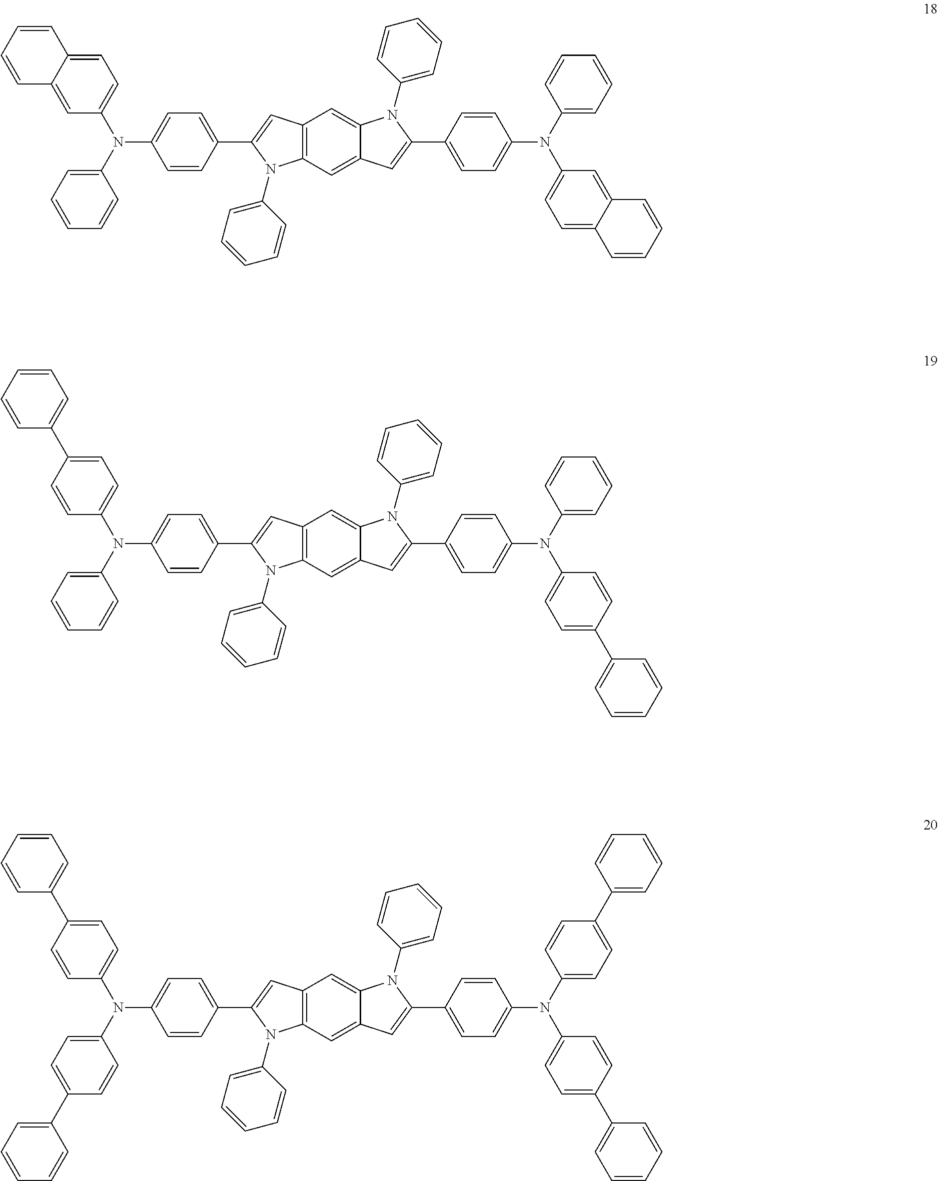 [SMILES: c1ccc(N(c2ccc(-c3cc4cc5c(cc(-c6ccc(N(c7ccccc7)c7ccc8ccccc8c7)cc6)n5-c5ccccc5)cc4n3-c3ccccc3)cc2)c2ccc3ccccc3c2)cc1 ; c1ccc(-c2ccc(N(c3ccccc3)c3ccc(-c4cc5cc6c(cc(-c7ccc(N(c8ccccc8)c8ccc(-c9ccccc9)cc8)cc7)n6-c6ccccc6)cc5n4-c4ccccc4)cc3)cc2)cc1 ; c1ccc(-c2ccc(N(c3ccc(-c4ccccc4)cc3)c3ccc(-c4cc5cc6c(cc(-c7ccc(N(c8ccc(-c9ccccc9)cc8)c8ccc(-c9ccccc9)cc8)cc7)n6-c6ccccc6)cc5n4-c4ccccc4)cc3)cc2)cc1]

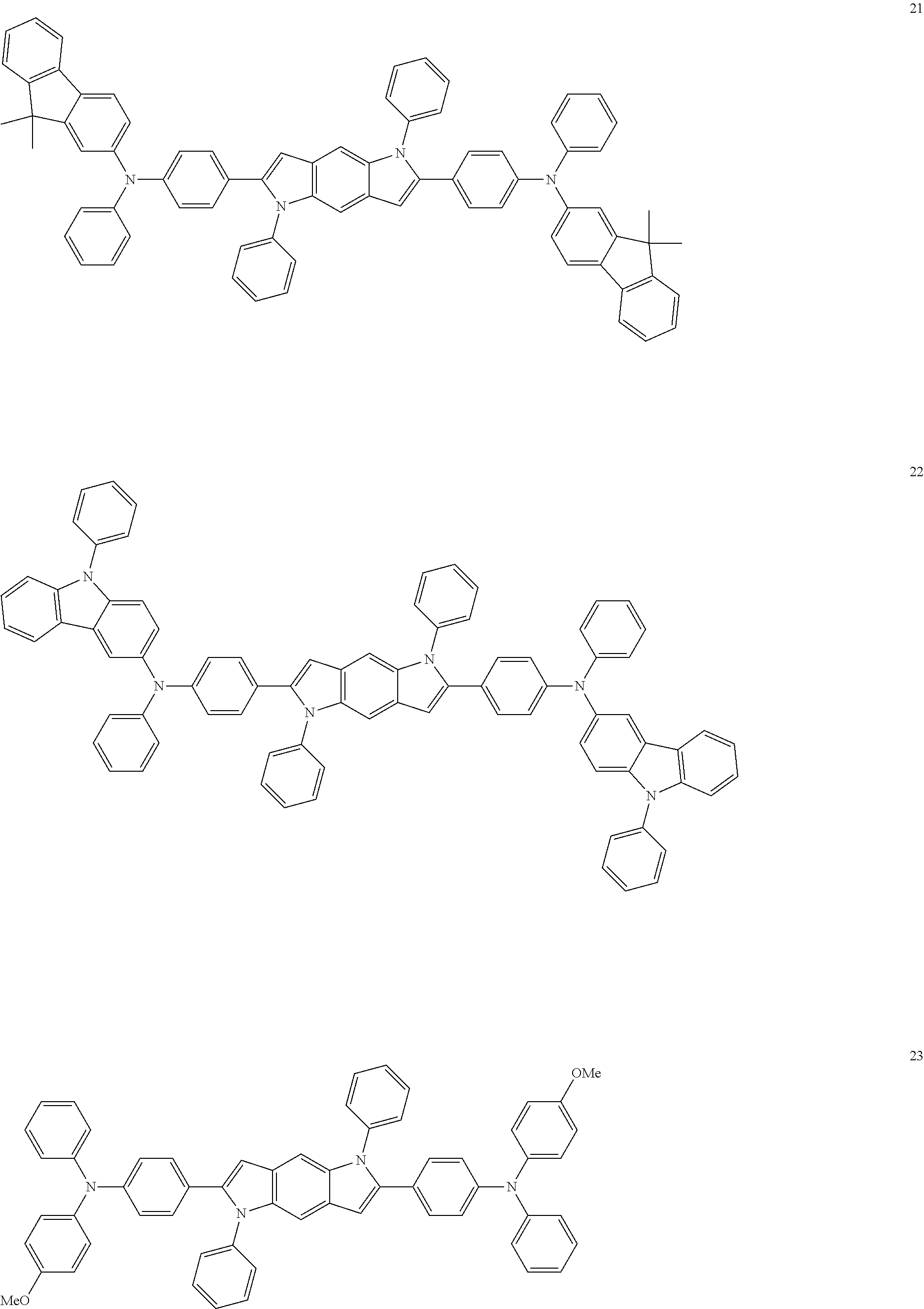

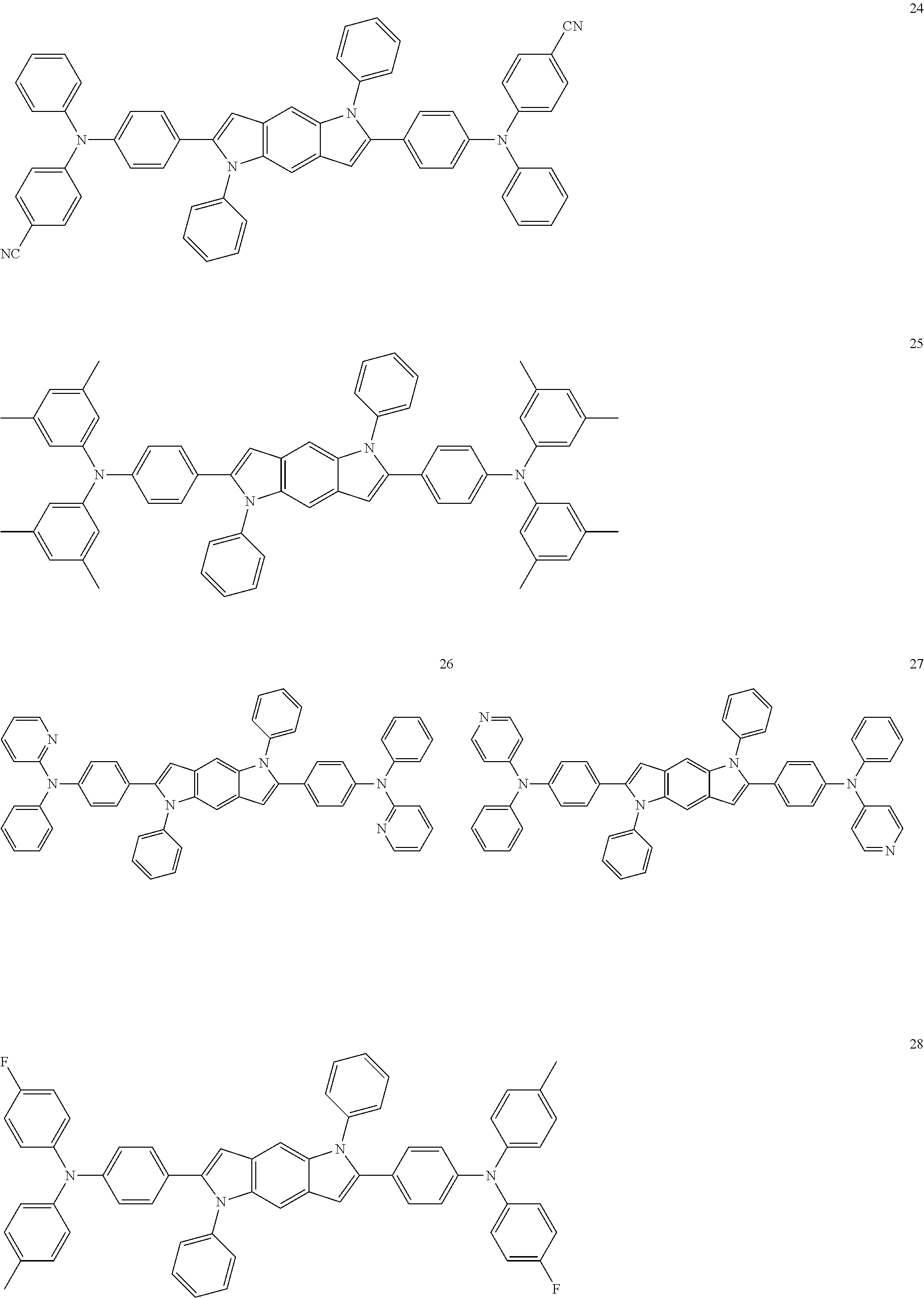

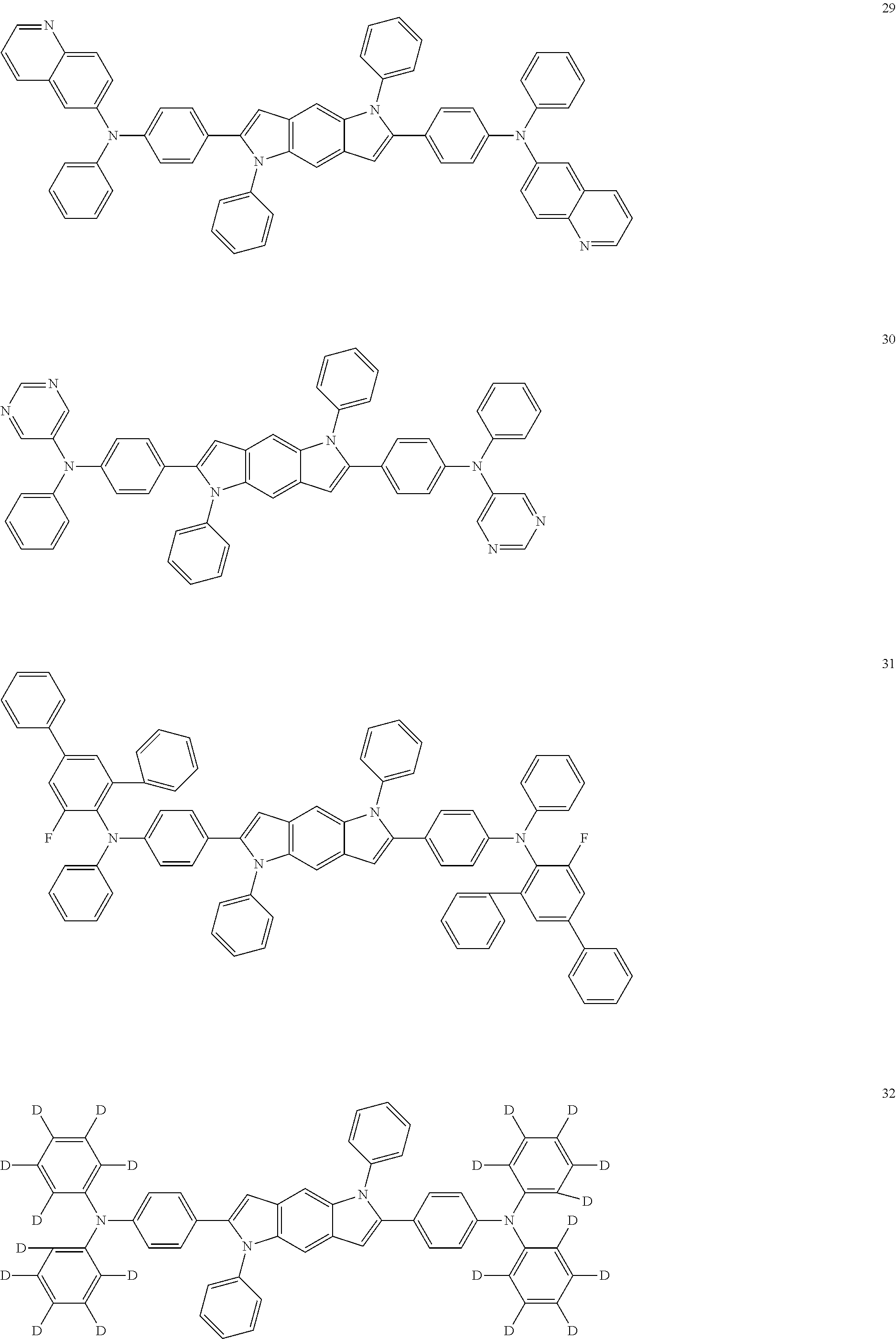

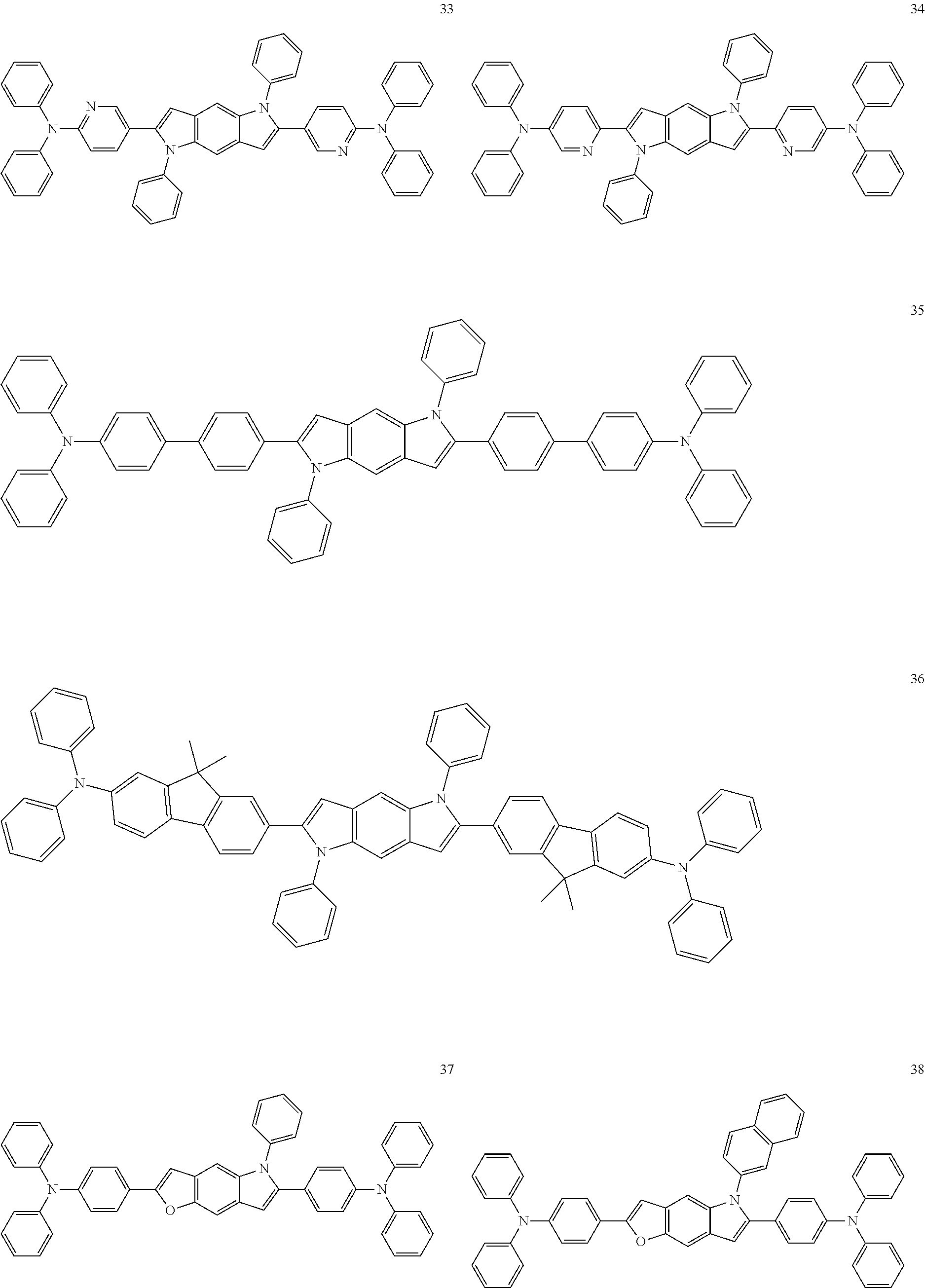

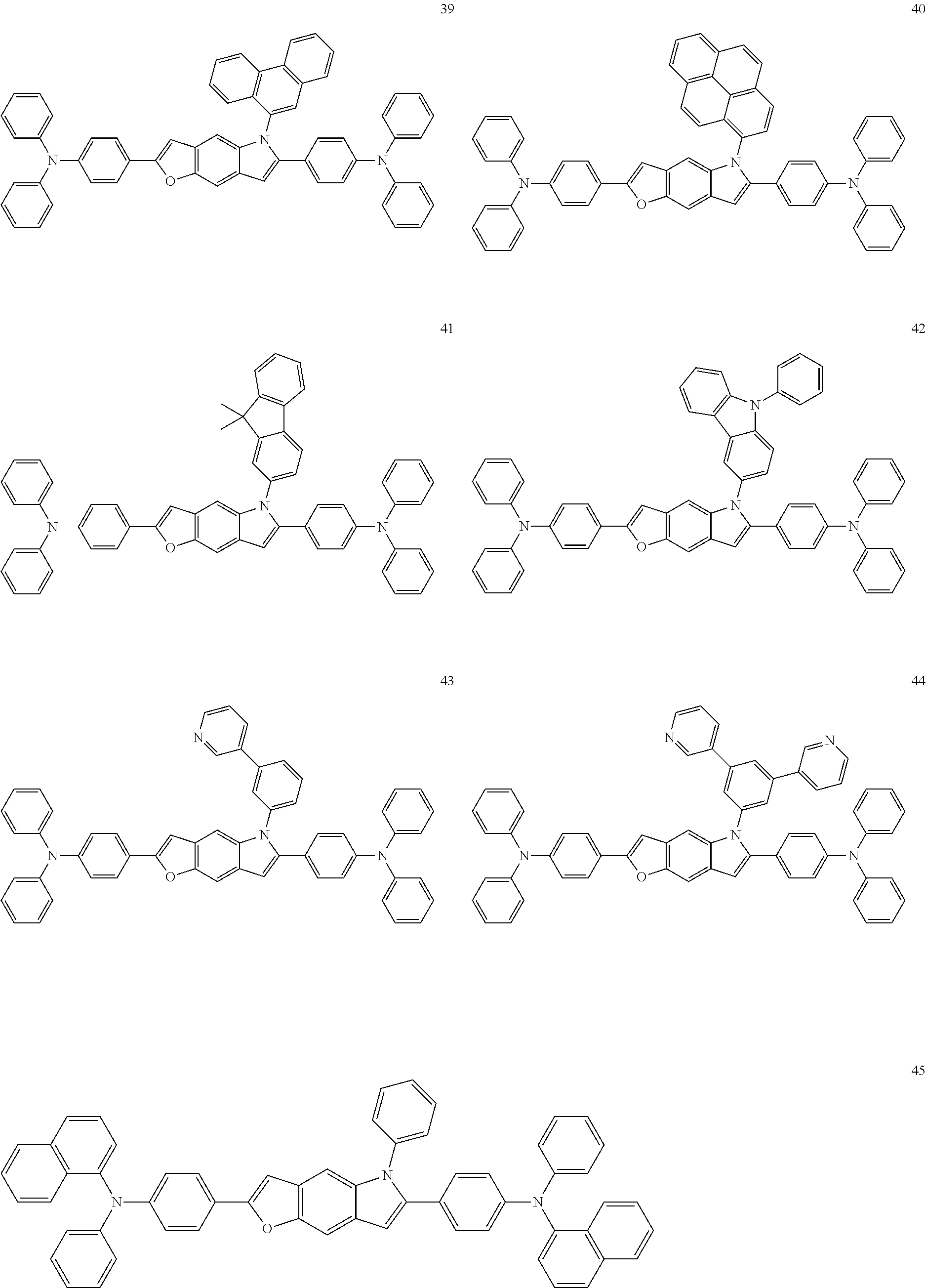

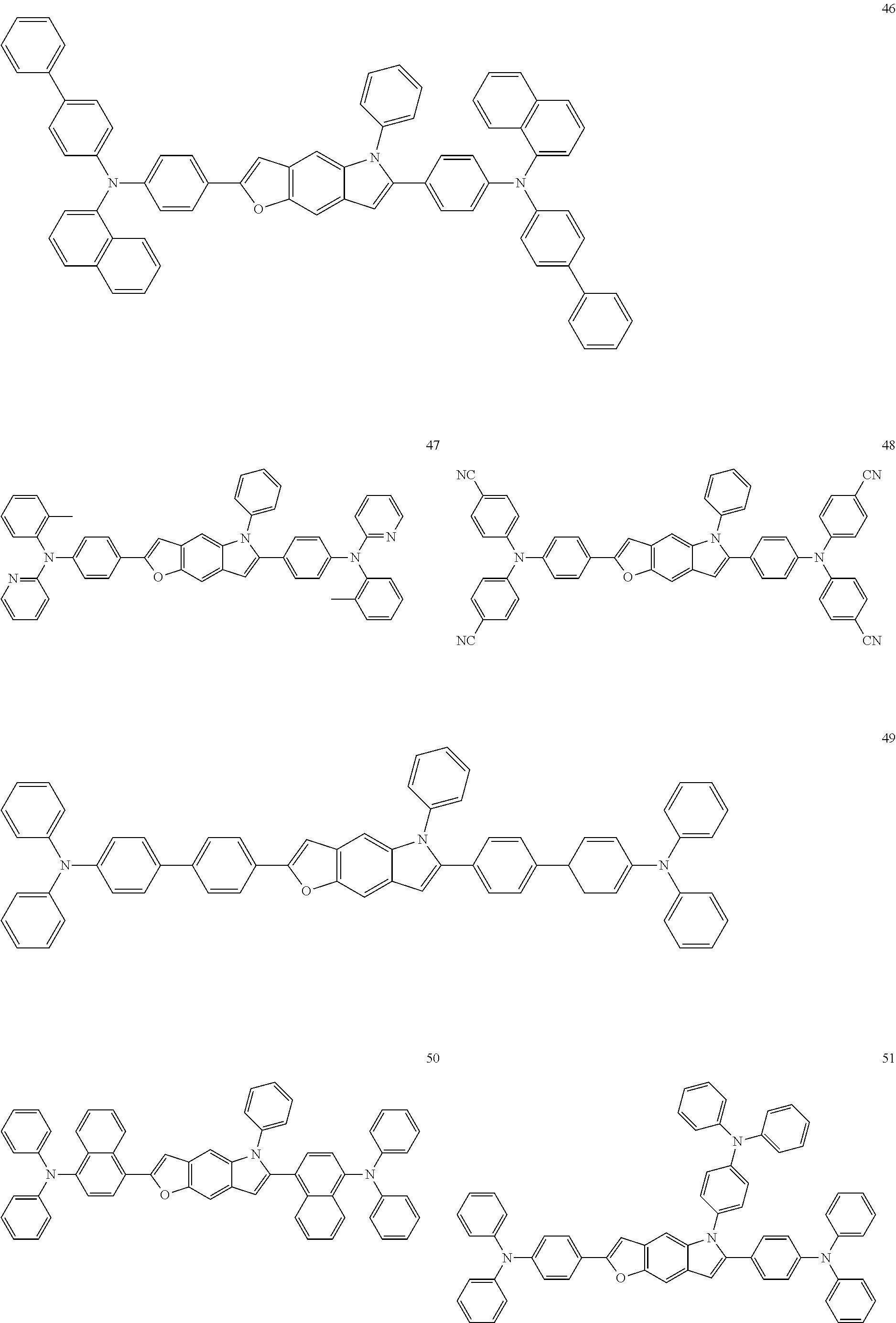

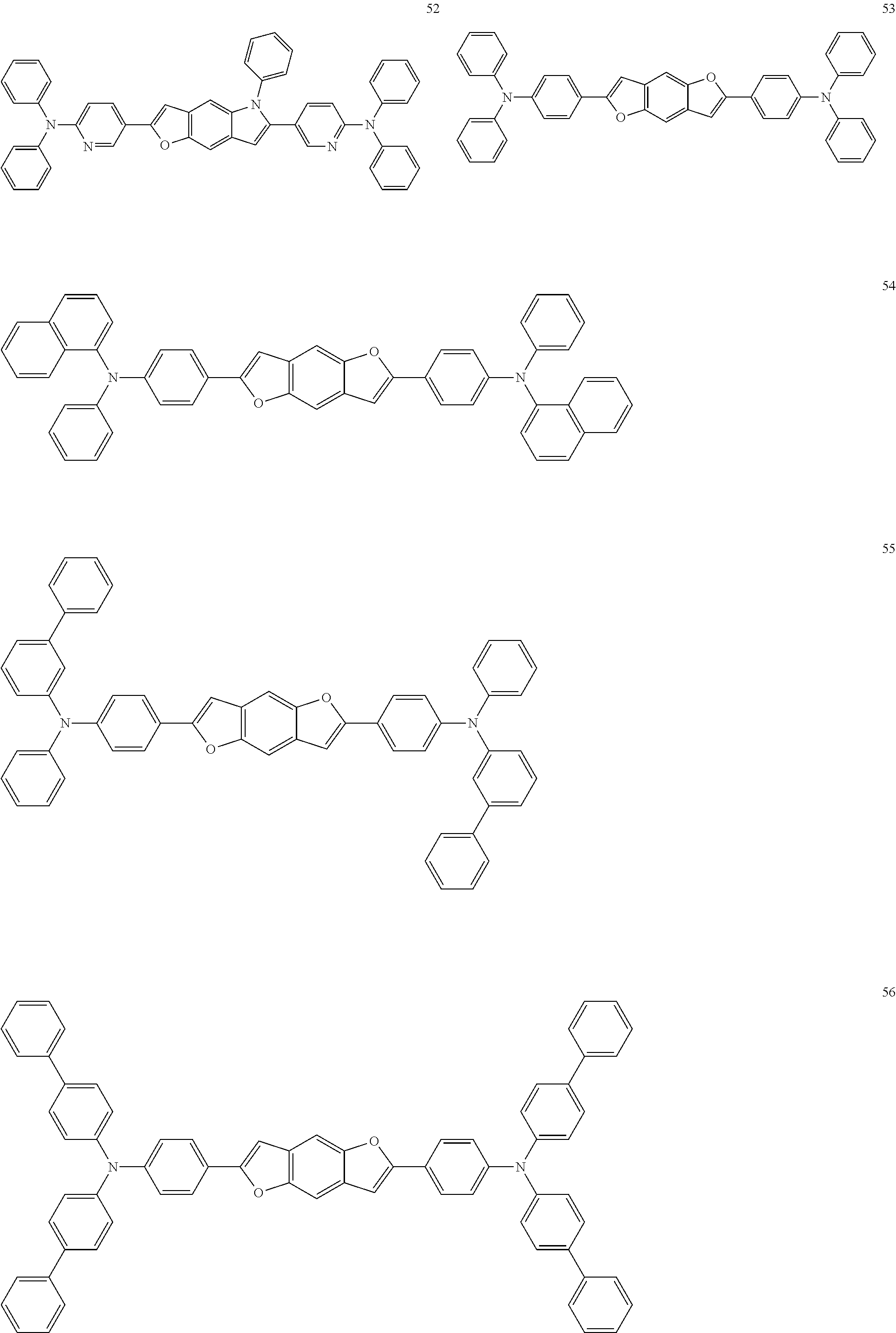

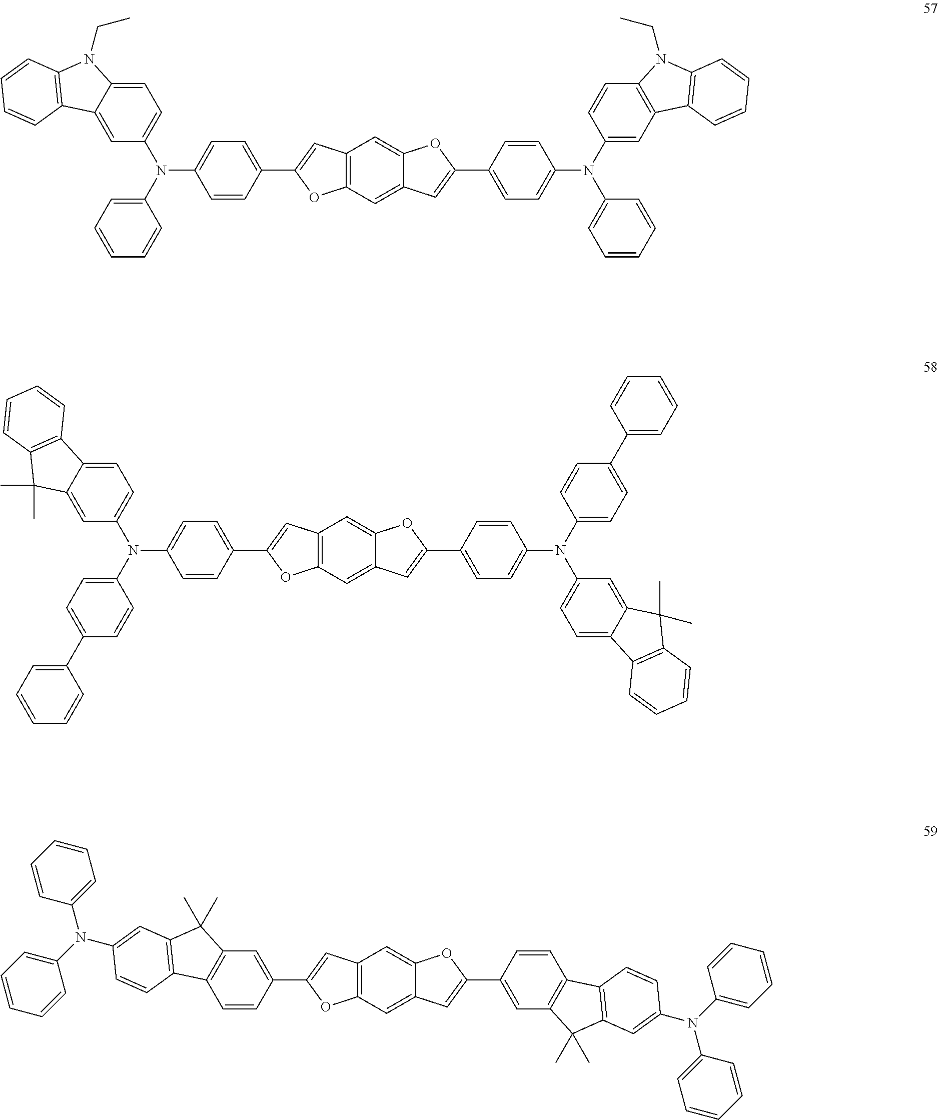

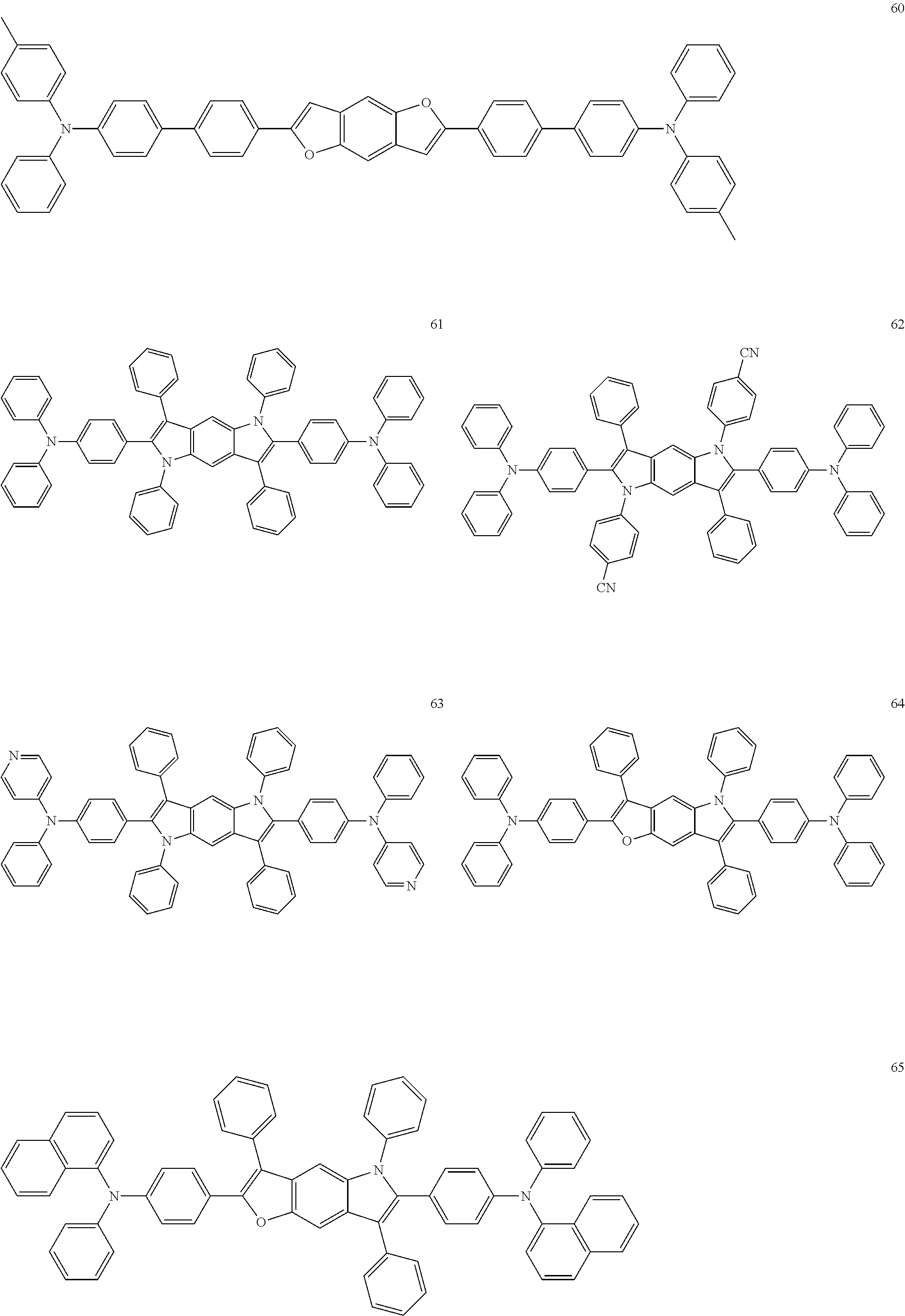

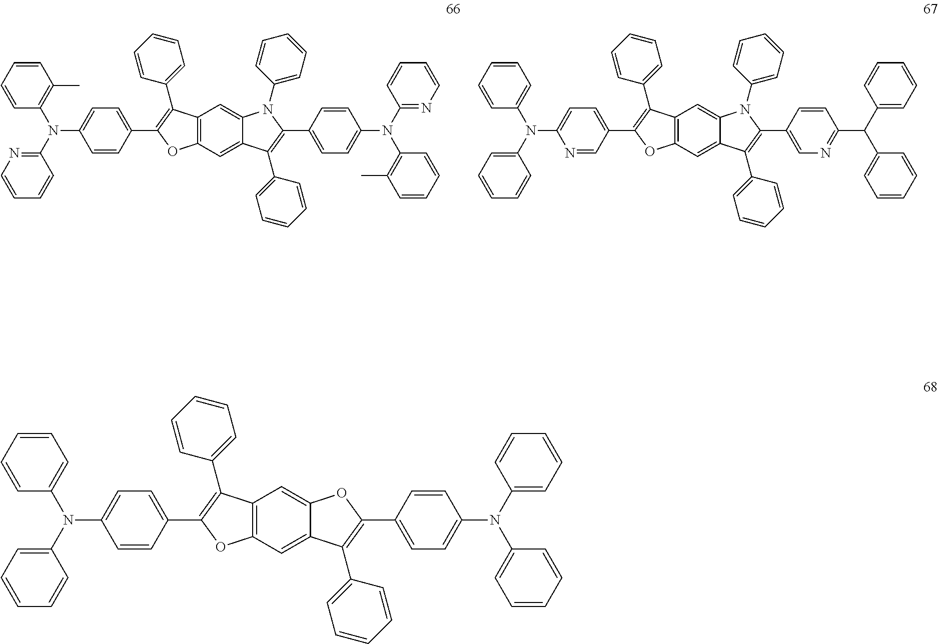 [SMILES: Cc1ccccc1N(c1ccc(-c2oc3cc4c(-c5ccccc5)c(-c5ccc(N(c6ccccn6)c6ccccc6C)cc5)n(-c5ccccc5)c4cc3c2-c2ccccc2)cc1)c1ccccn1 ; c1ccc(-c2c(-c3ccc(N(c4ccccc4)c4ccccc4)nc3)oc3cc4c(-c5ccccc5)c(-c5ccc(C(c6ccccc6)c6ccccc6)nc5)n(-c5ccccc5)c4cc23)cc1 ; c1ccc(-c2c(-c3ccc(N(c4ccccc4)c4ccccc4)cc3)oc3cc4c(-c5ccccc5)c(-c5ccc(N(c6ccccc6)c6ccccc6)cc5)oc4cc23)cc1]

Examples of the unsubstituted C₁-C₆₀ alkyl group (or C₁-C₆₀ alkyl group) may include C₁-C₆₀ linear or branched alkyl groups such as methyl, ethyl, propyl, isobutyl, sec-butyl, pentyl, iso-amyl, hexyl, and the like. The substituted C₁-C₆₀ alkyl group may be a group in which at least one hydrogen of the unsubstituted C₁-C₆₀ alkyl group is substituted with deuterium, a halogen atom, a hydroxyl group, a nitro group, a cyano group, an amino group, an amidino group, hydrazine, hydrazone, a carboxyl group or a salt thereof, a sulfonic acid group or a salt thereof, a phosphoric acid or a salt thereof, a C₁-C₆₀ alkyl group, a C₂-C₆₀ alkenyl group, a C₂-C₆₀ alkynyl group, a C₆-C₆₀ aryl group, a C₂-C₆₀ heteroaryl group, —N(Q₁₁)(Q₁₂), and —Si(Q₁₃)(Q₁₄)(Q₁₅) (wherein Q₁₁ through Q₁₅ may be each independently selected from hydrogen, a C₁-C₆₀ alkyl group, a C₂-C₆₀ alkenyl group, a C₂-C₆₀ alkynyl group, a C₅-C₆₀ aryl group, and a C₂-C₆₀ heteroaryl group).

The unsubstituted C₁-C₆₀ alkoxy group (or C₁-C₆₀ alkoxy group) may have a formula of —OA (in this regard, A is the unsubstituted C₁-C₆₀ alkyl group as described above) and examples thereof may include methoxy, ethoxy, isopropyloxy, and the like. At least one hydrogen atom of the unsubstituted C₁-C₆₀) alkoxy group may be substituted with the same substituent as in the substituted. C₁-C₆₀ alkyl group described above.

The unsubstituted C₂-C₆₀ alkenyl group (or C₂-C₆₀ alkenyl group) may be interpreted to contain at least one carbon-carbon double bond in the center or at a terminal of the unsubstituted C₂-C₆₀ alkyl group. Examples of the unsubstituted C₂-C₆₀ alkenyl group may include ethenyl, propenyl, butenyl, and the like. At least one hydrogen atom of the unsubstituted C₂-C₆₀ alkenyl group may be substituted with the same substituent as in the substituted C₁-C₆₀ alkyl group described above.

The unsubstituted C₂-C₆₀ alkynyl group (or C₂-C₆₀ alkynyl group) may be interpreted to contain at least one carbon-carbon triple bond in the center or at a terminal of the C₂-C₆₀ alkyl group defined above. Examples of the unsubstituted C₂-C₆₀ alkynyl group include ethynyl, propynyl, and the like. At least one hydrogen atom of the unsubstituted C₂-C₆₀ alkynyl group may be substituted with the same substituent as in the substituted C₁-C₆₀ alkyl group described above.

The unsubstituted C₅-C₆₀ aryl group may indicate a monovalent group having an aromatic carbocyclic system that has 5 to 60 carbon atoms and at least one aromatic ring and the unsubstituted C₅-C₆₀ arylene group indicates a divalent group having an aromatic carbocyclic system that has 5 to 60 carbon atoms and at least one aromatic ring. If the C₅-C₆₀ aryl group and the C₅-C₆₀ arylene group each independently have two or more aromatic rings, the rings may be fused with each other. At least one hydrogen atom of each of the unsubstituted C₅-C₆₀ aryl group and the unsubstituted C₅-C₆₀ arylene group may be substituted with the same substituent as in the substituted C₁-C₆₀ alkyl group described above.

Examples of the substituted or unsubstituted C₅-C₆₀ aryl group may include, but are not limited to, a phenyl group, a C₁-C₁₀ alkylphenyl group (e.g., an ethylphenyl group), a C₁-C₁₀ alkylbiphenyl group (e.g., an ethylbiphenyl group), a halophenyl group (e.g., an o-, m- and p-fluorophenyl group, and a dichlorophenyl group), a dicyanophenyl group, a trifluoromethoxyphenyl group, an o-, m-, and p-tolyl group, an o-, m- and p-cumenyl group, a mesityl group, a phenoxyphenyl group, an (α,α-dimethylbenzene)phenyl group, a (N,N′-dimethyl)aminophenyl group, a (N,N′-diphenyl)aminophenyl group, a pentalenyl group, an indenyl group, a naphthyl group, a halonaphthyl group (e.g., a fluoronaphthyl group), a C₁-C₁₀ alkylnaphthyl group (e.g., a methylnaphthyl group), a C₁-C₁₀ alkoxynaphthyl group (e.g., a methoxynaphthyl group), an anthracenyl group, an azulenyl group, a heptalenyl group, an acenaphthylenyl group, a phenalenyl group, a fluorenyl group, an anthraquinolyl group, a methylanthryl group, a phenanthryl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coroneryl group, a trinaphthylenyl group, a heptaphenyl group, a heptacenyl, a pyranthrenyl group, and an ovalenyl group. Examples of the substituted C₅-C₆₀ aryl group may be easily understood with reference to the examples of the unsubstituted C₅-C₆₀ aryl group described above and the substituents of the substituted C₁-C₆₀ alkyl group. Examples of the substituted or unsubstituted C₅-C₆₀ arylene group may be easily understood with reference to the substituted or unsubstituted C₅-C₆₀ aryl group described above.

The unsubstituted C₂-C₆₀ heteroaryl group may indicate a monovalent group having at least one aromatic ring system including carbon rings and at least one hetero atom selected from the group consisting of N, O, P, and S, and the unsubstituted C₂-C₆₀ heteroarylene group may indicate a divalent group having at least one aromatic ring system including carbon rings and at least one hetero atom selected from the group consisting of N, O, P, and S. In this regard, if the C₂-C₆₀ heteroaryl group and the C₂-C₆₀ heteroarylene group each independently have two or more aromatic rings, the rings may be fused with each other. At least one hydrogen atom of each of the unsubstituted C₂-C₆₀ heteroaryl group and the unsubstituted C₂-C₆₀ heteroarylene group may be substituted with the same substituents as in the C₁-C₆₀ alkyl group described above.

Examples of the unsubstituted C₂-C₆₀ heteroaryl group may include, but are not limited to, a pyrazolyl group, an imidazolyl group, an oxazolyl group, a thiazolyl group, a triazolyl group, a tetrazolyl group, an oxadiazolyl group, a pyridinyl group, a pyridazinyl group, a pyrimidinyl group, a triazinyl group, a carbazolyl group, an indolyl group, a quinolinyl group, an isoquinolinyl group, a benzoimidazolyl group, an imidazopyridinyl group, and an imidazopyrimidinyl group. Examples of the unsubstituted C₂-C₆₀ heteroarylene group may be easily understood with reference to the examples of the substituted or unsubstituted C₂-C₆₀ arylene group.

The substituted or unsubstituted C₅-C₆₀ aryloxy group may have a formula of —OA₂ wherein A₂ is the substituted or unsubstituted C₅-C₆₀ aryl group as described above, and the substituted or unsubstituted C₅-C₆₀ arylthio group may have a formula of —SA₃ wherein A₃ is the substituted or unsubstituted C₅-C₆₀ aryl group described above.

The condensed-cyclic compound of Formula 1 has a rigid core structure with three rings such as

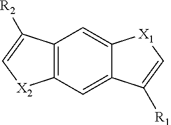

and thus may have high glass transistion temperature and a high melting point. Thus, when an organic light-emitting diode (OLED) including the condensed-cyclic compound of Formula 1 is stored and/or operated, the OLED may have high resistance to Joule's heat generated between organic layers, inside the organic layers, and/or between one of the organic layers and one of the electrodes. Therefore, an OLED including the condensed-cyclic compound of Formula 1 may have long lifetime.

In addition, as shown in Formula 1′, A-site carbon and B-site carbon are linked to —N(Ar₃)(Ar₄) and —N(Ar₁)(Ar₂) via L₂ and L₁, respectively and thus an overall structure of the condensed-cyclic compound of Formula 1 may be in a conjugated form. In Formula 1, the core and rings of L₁ and L₂ are twisted with each other and thus the condensed-cyclic compound of Formula 1 may provide an emission spectrum that is shifted to a shorter wavelength than a compound not including L₁ and L₂ and may exhibit a high color purity of blue emission, accordingly.

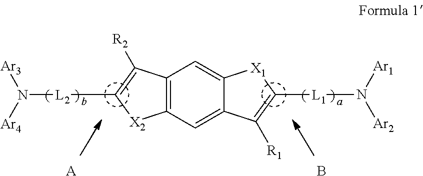

The chromaticity coordinates defined by National Television System Committee (NTSC) are R(0.67, 0.33), G(0.21, 0.71), and B(0.14, 0.08), and an area of the chromaticity coordinates is 0.158. Thus, to create an image of colors that are close to natural colors by increasing a color reproduction rate of an OLED, a high color purity of blue that is closed to B(0.14, 0.08) defined by NTSC is needed. Since the condensed-cyclic compound of Formula 1 exhibts the high color purity of blue emission as described above, an OLED manufactured using the same may be a high-quality, full-color OLED.

The condensed-cyclic compound of Formula 1 may be synthesized using a known organic synthesis method. The synthesis method of the condensed-cyclic compound of Formula 1 may be easily understood by one of ordinary skill in the art with reference to Examples, which will be described later.

The condensed-cyclic compound of Formula 1 may be used between a pair of electrodes of an OLED. For example, the condensed-cyclic compound of Formula 1 may be used in an emission layer (EML) and/or a layer between an anode and the EML. The layer may be a hole injection layer (HIL), a hole transport layer (HTL), or a functional layer having hole injection and hole transport abilities.

According to another embodiment of the present invention, there is provided an OLED including a first electrode, a second electrode facing the first electrode, and an organic layer interposed between the first electrode and the second electrode, wherein the organic layer may include at least one of the condensed-cyclic compounds of Formula 1 as described above.

The expression “the organic layer may include at least one of the condensed-cyclic compounds of Formula 1” as used herein means that the organic layer includes one of the condensed-cyclic compounds represented by Formula 1 above or at least two different condensed-cyclic compounds selected from the condensed-cyclic compounds represented by Formula 1 above.

For example, the organic layer may include only Compound 3 as the condensed-cyclic compound. In this regard, Compound 3 may be included in a HTL of the OLED (refer to Example 1 below). Also, the organic layer may include Compound 19 and Compound 41 as the condensed-cyclic compounds. In this regard, Compounds 19 and 41 may be included in same layer (e.g., in an EML) or in different layers (e.g., Compound 19 may be included in a HTL and Compound 41 may be included in an EML, refer to Example 7 below).

The organic layer may include at least one of a HIL, a HTL, a functional layer having hole injection and hole transport abilities (hereinafter, referred to as “H-funcational layer”), a buffer layer, an electron blocking layer (HBL), an emission layer, a hole blocking layer (HBL), an electron transport layer (ETL), an electron injection layer (EIL), and a functional layer having electron transport and electron injection abilities (hereinafter, referred to as “E-functional layer”).

The term “organic layer” used herein refers to a single layer or multiple layers interposed between the first electrode and the second electrode.

For example, the organic layer may include an EML including the condensed-cyclic compound of Formula 1. Also, the organic layer may include at least one of a HIL, a HTL, and an H-fuctional layer and the condensed-cyclic compound of Formula 1 may be included in at least one of the HIL, the HTL, and the H-functional layer.

The condensed-cyclic compound of Formula 1 included in the EML may act as a fluorescent dopant. For example, the condensed-cyclic compound of Formula 1 may act as a blue fluorescent dopant that emits bule light. Alternatively, the condensed-cyclic compound of Formula 1 may act as a fluorescent or phosphorescent host for emitting red light, green light, or blue light.

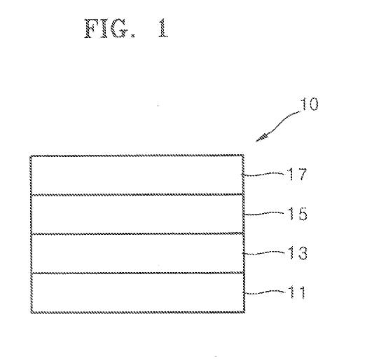

FIG. 1 is a schematic cross-sectional view of an OLED 10 according to an embodiment of the present invention. Hereinafter, structure and manufacturing method of an OLED will be described in more detail with reference to FIG. 1.

A substrate 11 may be a substrate used in a general OLED, and may be a glass substrate or a transparent plastic substrate having excellent mechanical strength, thermal stability, transparency, surface smoothness, ease of handling, and waterproofness.

A first electrode 13 may be formed by applying a first electrode material on the substrate 11 by deposition or sputtering. When the first electrode 13 is an anode, the first electrode material may be selected from materials having a high work function so as to facilitate hole injection. The first electrode 13 may be a reflective electrode or a transparent electrode. Examples of the first electrode material may include indium-tin oxide (ITO), Indium-zinc-oxide (IZO), tin oxide (SnO₂), and zinc oxide (ZnO). Also, when magnesium (Mg), aluminum (Al), aluminum-lithium (Al—Li), calcium (Ca), magnesium-indium (Mg—In), or magnesium-silver (Mg—Ag) is used as the first electrode material, the first electrode 13 may be formed as a reflective electrode.

The first electrode 13 may be formed as a single layer or have a multi-layered structure having at least two layers. For example, the first electrode 13 may have a three-layered structure, e.g., ITO/Ag/ITO, but is not limited thereto.

An organic layer 15 is formed on the first electrode 13.

The organic layer 15 may include a HIL, a HTL, a buffer layer, an EML, an ETL, and an EIL.

The HIL may be formed on the first electrode 13 by using various methods such as vacuum deposition, spin coating, casting, or LB deposition.

When the HIL is formed by vacuum deposition, the deposition conditions may vary according to a compound used as a Material for forming the HIL, a structure of a desired HIL, and thermal characteristics. For example, the deposition condition may be, but is not limited to a deposition temperature of about 100° C. to about 500° C., a degree of vacuum of about 10⁻⁸ torr to about 10⁻³ torr, and a deposition speed of about 0.01 to about 100 Å/sec.

When the HIL is formed by spin coating, the coating condition may vary according to a compound used as a material for forming the HIL, a structure of a desired HIL, and thermal characteristics. For example, the coating condition may be, but is not limited to, a coating speed of about 2,000 rpm to about 5,000 rpm and a heat treatment temperature for removing a solvent after coating of about 80° C. to about 200° C.

The material for forming the HIL may be a known hole injection material. Examples of the known hole injection material include, but are limited to, N,N′-diphenyl-N,N′-bis-[4-(phenyl-m-tolyl-amino)-phenyl]-biphenyl-4,4′-diamine (DNTPD), a phthalocyanine compound such as copper phthalocyanine, 4,4′,4″-tris(3-methylphenylphenylamino)triphenylamine (m-MTDATA), N,N′-di(1-naphthyl)-N,N′-diphenylbenzidine (NPB), TDATA, 2-TNATA, polyaniline/dodecylbenzenesulfonic acid (PANI/DBSA), poly(3,4-ethylenedioxythiophene)/poly(4-styrenesulfonate) (PEDOT/PSS), polyaniline/camphor sulfonicacid (PANI/CSA), and polyaniline/poly(4-styrenesulfonate) (PANI/PSS):

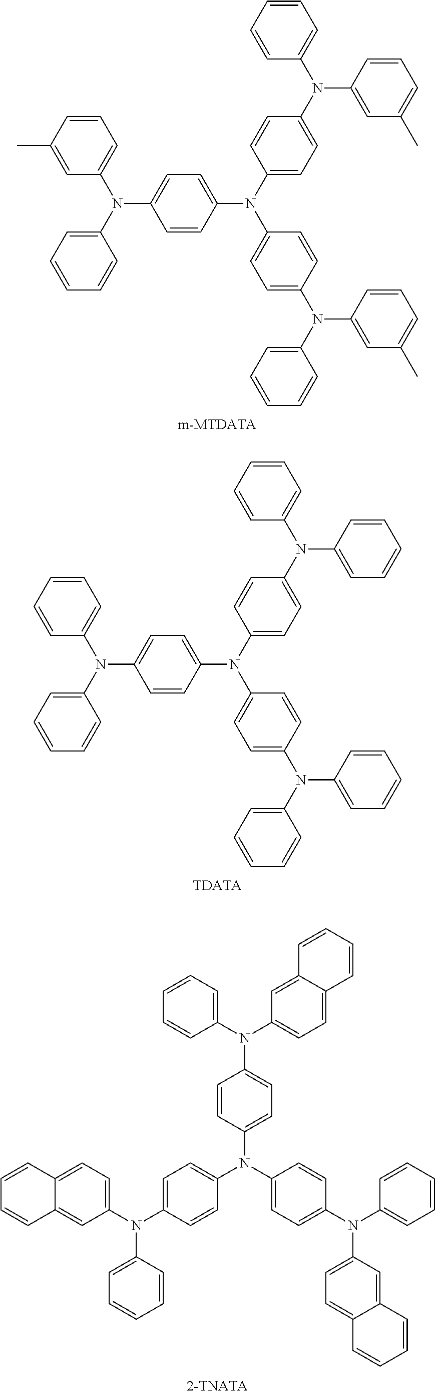

The thickness of the HIL may be in the range of about 100 Å to about 10,000 Å. In some embodiments, the thickness of the HIL may be in the range of about 100 Å to about 1,000 Å. When the thickness of the HIL is within these ranges, satisfactory hole injection properties may be obtained without a substantial increase in driving voltage.

Next, an HTL may be formed on the HIL by using various methods such as vacuum deposition, spin coating, casting, or LB deposition. When the HTL is formed by vacuum deposition or spin coating, the deposition and coating conditions may vary according to a used compound. However, in general, the deposition and coating conditions may be almost the same as the condition for forming the HIL.

As a material for forming the HTL, at least one of the condensed-cyclic compounds of Formula 1, and a known hole transporting material are used. Examples of the known hole transporting material may include, but are not limited to, carbazole derivatives such as N-phenylcarbazole and polyvinylcarbazole, N,N′-bis(3-methylphenyl)-N,N′-diphenyl-[1,1-biphenyl]-4,4′-diamine (TPD), 4,4′,4″-tris(N-carbazolyl)triphenylamine (TCTA), and N,N′-di(1-naphthyl)-N,N′-diphenylbenzidine (NPB).

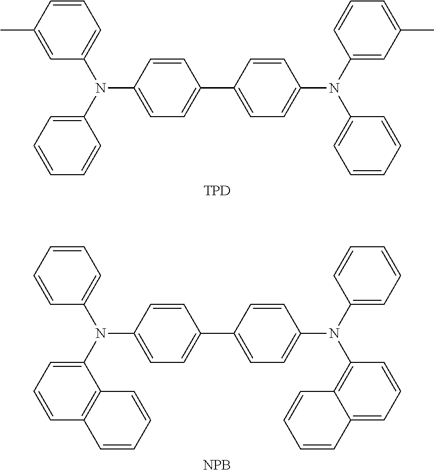

The thickness of the HTL may be in the range of about 50 Å to about 2,000 Å. In some embodiments, the thickness of the HTL may be in the range of about 100 Å to about 1,500 Å. When the thickness of the HTL is within these ranges, satisfactory hole transport properties may be obtained without a substantial increase in driving voltage.

At least one of the hole injection material and the hole transporting material as described above may be included in the H-functional layer. The thickness of the H-funcational layer may be in the range of about 500 Å to about 10,000 Å. In some embodiments, the thickness of the H-funcational layer may be in the range of about 100 Å to about 1,000 Å. When the thickness of the H-functional layer is within these ranges, satisfactory hole injection and hole transport properties may be obtained without a substantial increase in driving voltage.

At least one of the HIL, the HTL, and the H-functional layer may include at least one of compounds represented by Formula 300 and Formula 350 below:

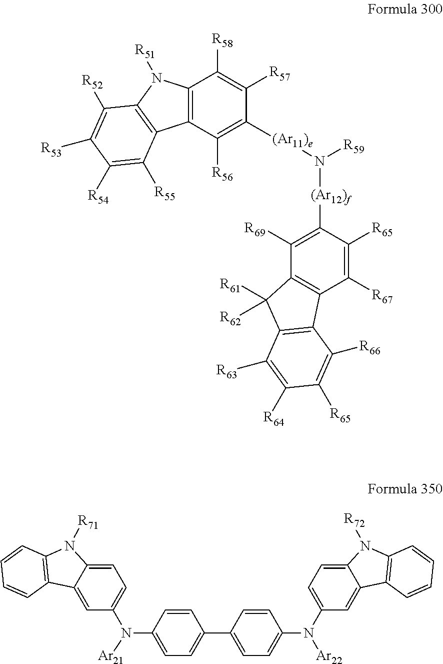

Wherein Ar₁₁, Ar₁₂, Ar₂₁, and Ar₂₂ may be each independently a substituted or unsubstituted C₅-C₆₀ arylene group. A detailed description of Ar₁₁, Ar₁₂, Ar₂₁, and Ar₂₂ may be found in the detailed description of L₁ above.

In Formula 300 above, e and f may be each independently an integer of 0 to 5. In some emdodiments, e and f may be each independently 0, 1, or 2. For example, e may be 1 and f may be 0, however, e and f are not limited to the above example.

In Formulae 300 and 350 above, R₅₁ through R₅₈, R₆₁ through R₆₉, and R₇₁ and R₇₂ may be each independently hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ aryl group, a substituted or unsubstituted C₅-C₆₀ aryloxy group, or a substituted or unsubstituted C₅-C₆₀ arylthio group.

For example, R₅₁ through R₅₈, R₆₁ through R₆₉, and R₇₁ and R₇₂ may be each independently one of hydrogen; deuterium; a halogen atom; a hydroxyl group; a cyano group; a nitro group; an amino group; an amidino group; hydrazine; hydrazone; a carboxyl group or a salt thereof; a sulfonic acid group or a salt thereof; a phosphoric acid or a salt thereof; a C₁-C₁₀ alkyl group (e.g., methyl, ethyl, propyl, butyl, pentyl, hexyl, and the like), a C₁-C₁₀ alkoxy group (e.g., methoxy, ethoxy, propoxy, butoxy, pentoxy, and the like); a C₁-C₁₀ alkyl group and a C₁-C₁₀ alkoxy group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof; a phenyl group; a naphthyl group; an anthryl group; a fluorenyl group; a pyrenyl group; and a phenyl group, a naphthyl group, an anthryl group, a fluorenyl group, and a pyrenyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, and a C₁-C₁₀ alkoxy group, but are not limited thereto.

In Formula 300 above, R₅₉ may be one of a phenyl group; a naphthyl group; an anthryl group; a biphenyl group; a pyridinyl group; and a phenyl group, a naphthyl group, an anthryl group, a biphenyl group, and a pyridinyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a substituted or unsubstituted C₁-C₂₀ alkyl group, and a a substituted or unsubstituted alkoxy group.

The compound of Formula 300 may be represented by Formula 300A below, but is not limited thereto:

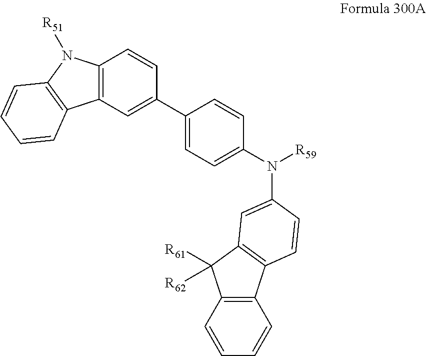

Wherein a detailed description of R₅₁, R₆₀, R₆₁, and R₅₉ may be the same as already provided above.

For example, at least one of the HIL, the HTL, and the H-functional layer may be include at least one of Compounds 301 through 320 below, but is not limited thereto:

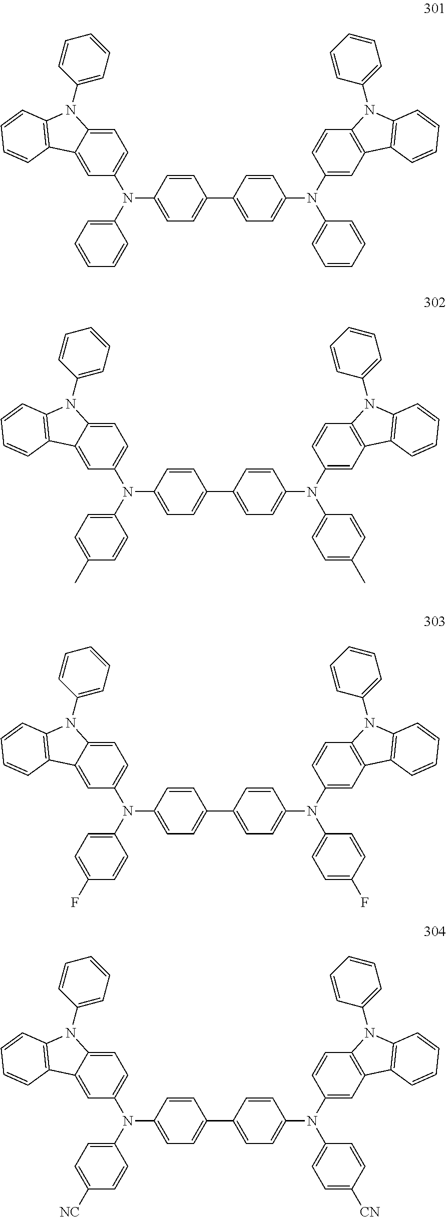 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1]

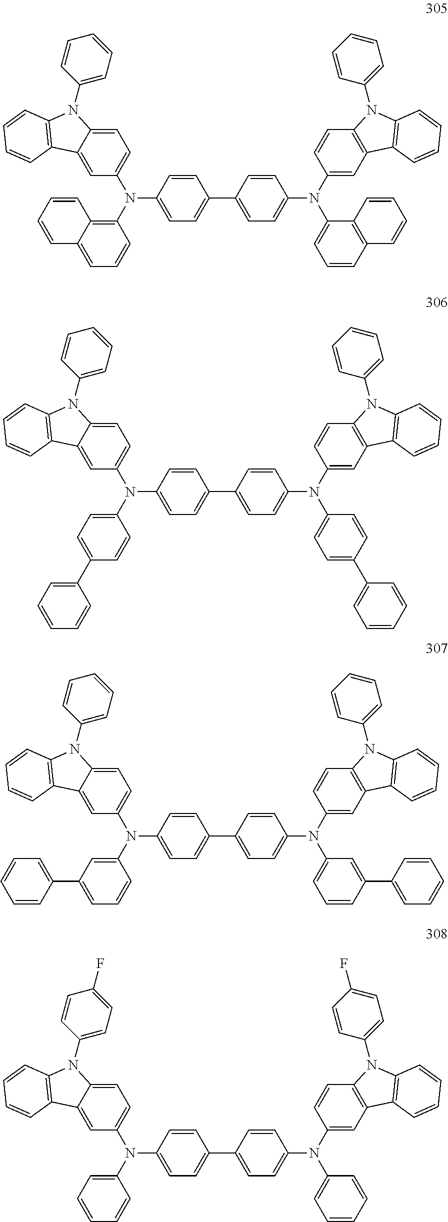 [SMILES: c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

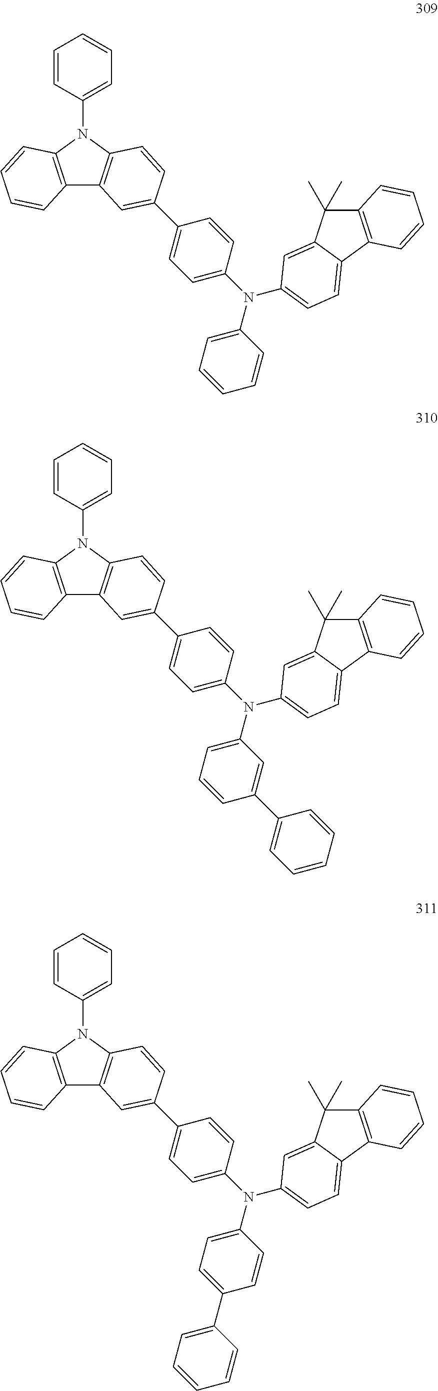 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

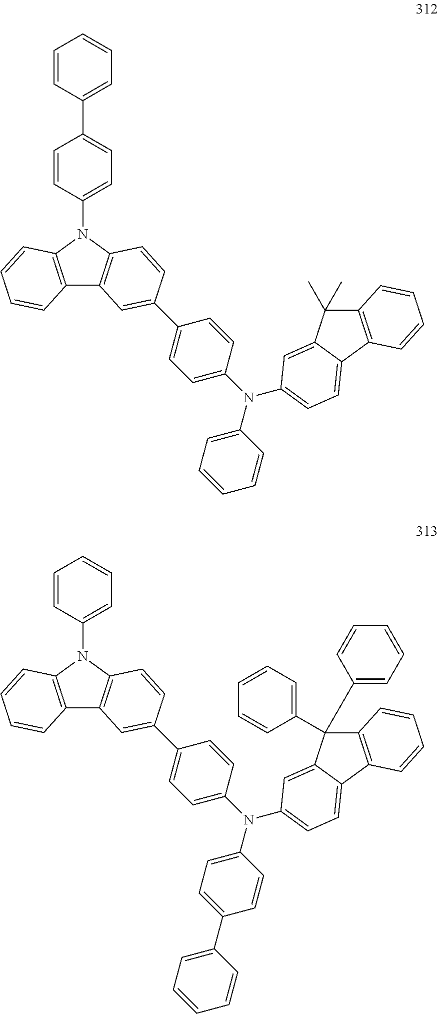 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

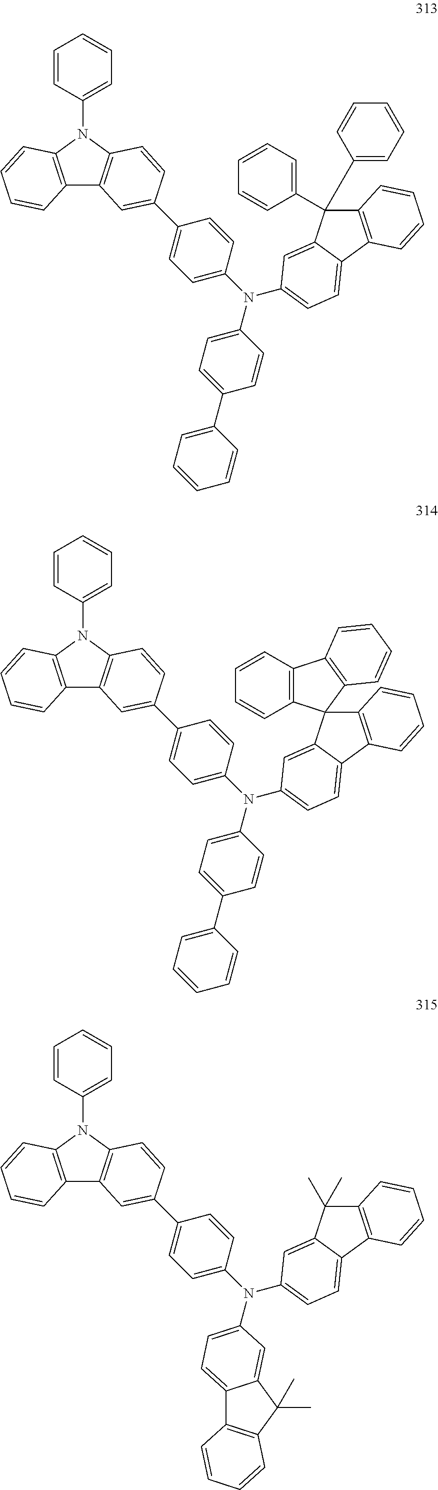 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21]

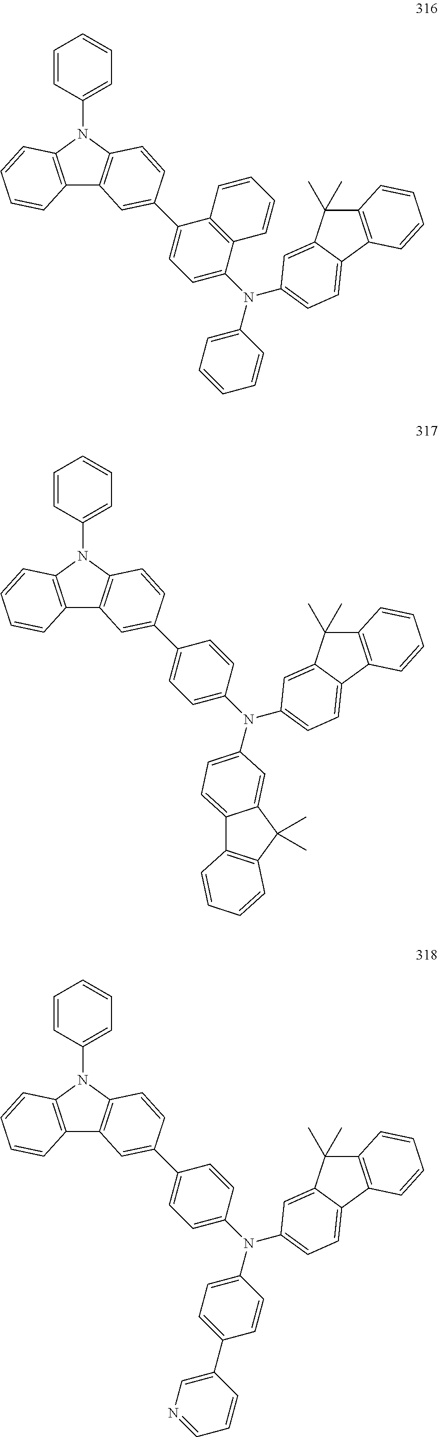 [SMILES: CC1(C)c2ccccc2-c2ccc(N(c3ccccc3)c3ccc(-c4ccc5c(c4)c4ccccc4n5-c4ccccc4)c4ccccc34)cc21 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccc(-c4cccnc4)cc3)c3ccc(-c4ccc5c(c4)c4ccccc4n5-c4ccccc4)cc3)cc21]

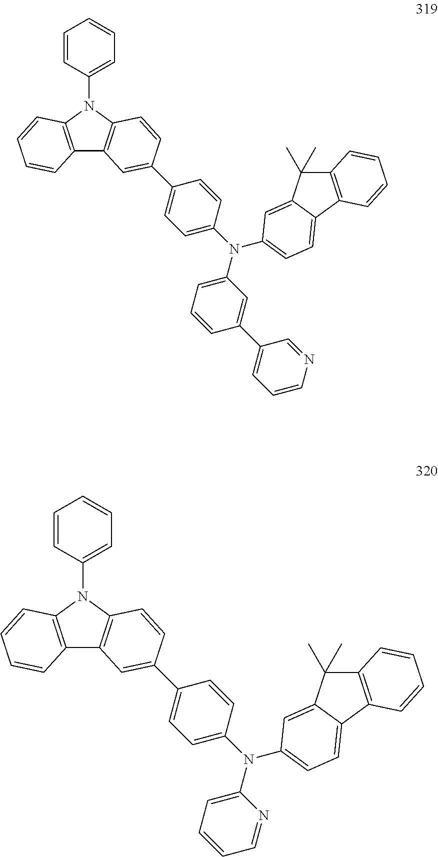 [SMILES: CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21]

At least one of the HIL, the HTL, and the H-functional layer may further include a charge-generating material so as to increase the conductive of the layers, in addition to the hole injection material, the hole transporting material and/or the material for forming the H-functional layer having hole injection and hole transport abilities.

The charge-generating material may be, for example, a p-dopant. The p-dopnat may be one of a quinine derivative, a metal oxide, and a cyano-containing compound, but is not limited thereto. Examples of the p-dopant may include, but are not limited to, quinone derivatives such as tetra-cyanoquinodimethane (TCNQ) and 2,3,5,6-tetrafluoro-tetracyano-1,4-benzoquinodimethane (F4-CTNQ); metal oxides such as an tungsten oxide and a molybdenum oxide; and cyano-containing compounds such as Compound 200 below and the like.

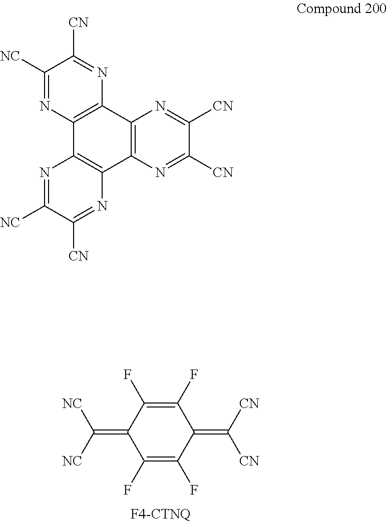 [SMILES: N#Cc1nc2c3nc(C#N)c(C#N)nc3c3nc(C#N)c(C#N)nc3c2nc1C#N ; N#CC(C#N)=c1c(F)c(F)c(=C(C#N)C#N)c(F)c1F]

When the HIL, the HTL, or the H-functional layer further includes the charge-generating material, the charge-generating material may be homogeneously or inhomogeneously dispersed in the HIL, the HTL, or the H-functional layer.

A buffer layer may be interposed between the EML and at least one of the HIL, the HTL, and the H-functional layer. The buffer layer increases efficiency by compensating for an optical resonance distance according the wavelength of light emiited from the EML. The buffer layer may include the hole injection material and the hole transporting material described ablve. Also, the buffer layer may include the same material as one of the materials included in the HIL, the HTL, and the H-functional layer.

An EML may be formed on the HTL, the H-functional layer, or the buffer layer by vacuum deposition, spin coating, casting, or LB deposition. When the EML is formed by vacuum deposition or spin coating, the deposition and coating conditions may vary according to a used compound. However, in general, the deposition and coating, conditions may be almost the same as the condition for forming the HIL.

The EML may include at least one of the condensed-cyclic compounds of Formula 1.

The EML may further include a host.

Examples of the host may include, but are not limited to, Tris(8-hydroxyquinolinato)aluminium (Alq3), 4,4′-N,N′-dicabazole-biphenyl (CBP), poly(n-vinylcabazole) (PVK), 9,10-di(naphthalene-2-yl)anthracene (ADN), 4,4′,4″-tris(N-carbazolyl)-triphenylamine(TCTA), 1,3,5-tris(N-phenylbenzimidazole-2-yl)benzene (TPBI), 3-tert-butyl-9,10-di(naphth-2-yl) anthracene (TBADN), E3, and distyrylarylene (DSA), dmCBP (refer to Formula below), and Compounds 501 through 509 below.

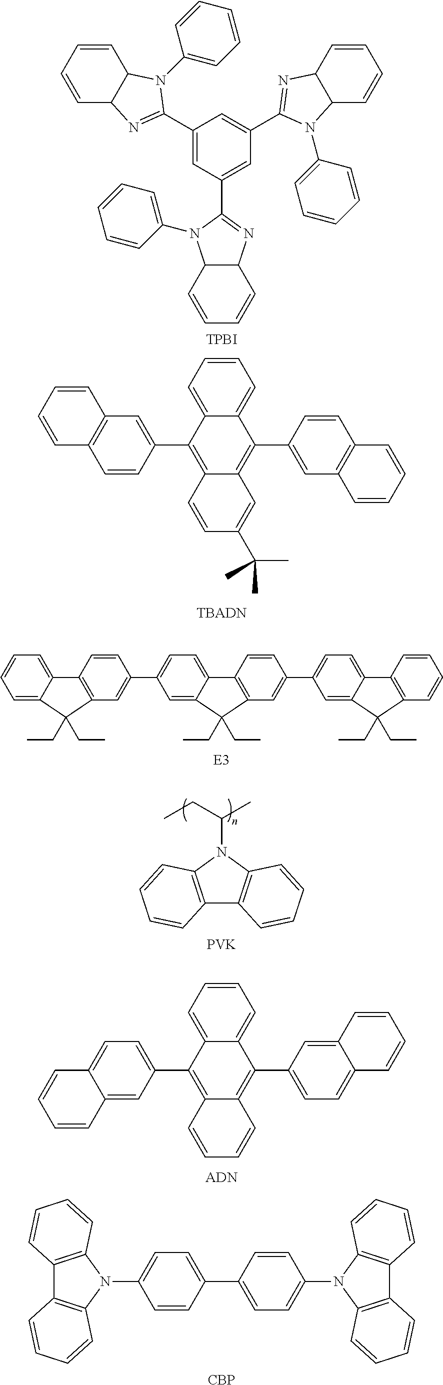

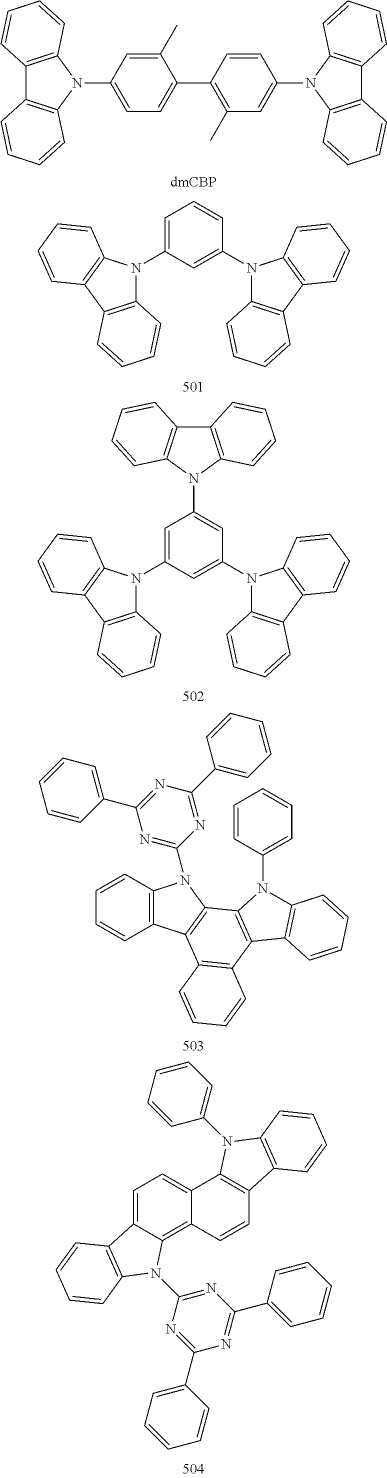

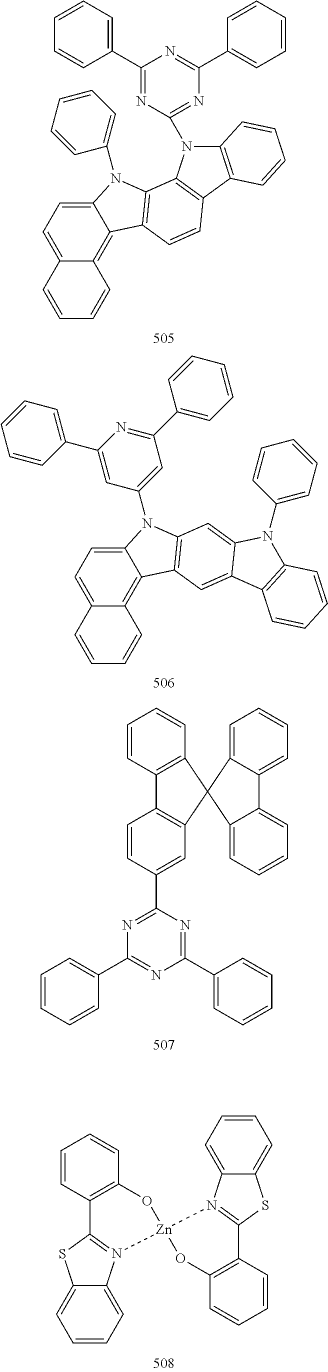

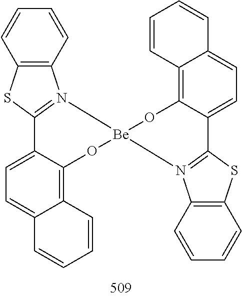

Also, the host may be an anthracene-based compound represented by Formula 400 below:

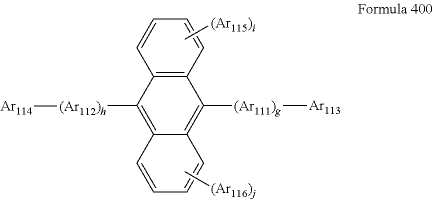

Wherein Ar₁₁₁ and Ar₁₁₂ may be each independently a substituted or unsubstituted C₅-C₆₀ arylene group; Ar₁₁₃ through Ar₁₁₆ may be each independently a substituted or unsubstituted C₁-C₁₀ alkyl group or a substituted or unsubstituted C₅-C₆₀ aryl group; and g, h, i, and j may be each independently an integer of 0 to 4.

For example, in Formula 400 above, Ar₁₁₁ and Ar₁₁₂ may be each independently one of a phenylene group; a naphthylene group; a phenanthrenylene group; a pyrenylene group; and a phenylene group, a naphthylene group, a phenanthrenylene group, a fluorenyl group, and a pyrenylene group that are substituted with at least one of a phenyl group, a naphthyl group, and an anthryl group.

In Formula 400 above, g, h, i, and j may be each independently 0, 1, or 2.

In Formula 400 above, Ar₁₁₃ through Ar₁₁₆ may be each independently, but are not limited to, a C₁-C₁₀ alkyl group that is substituted with at least one of a phenyl group, a naphthyl group, and an anthryl group; a phenyl group; a naphthyl group; an anthryl group; a pyrenyl group; a phenanthrenyl group; a fluorenyl group; a phenyl group, a naphthyl group, an anthryl group, a pyrenyl group, a phenanthrenyl group, and a fluorenyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a phenyl group, a naphthyl group, an anthryl group, a pyrenyl group, a phenanthrenyl group, and a fluorenyl group; and

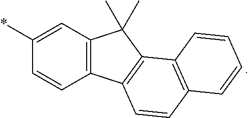 [SMILES: *c1ccc2c(c1)C(C)(C)c1c-2ccc2ccccc12]

For example, the anthracene-based compound of Formula 400 may be, but is not limited to, one of the compounds below:

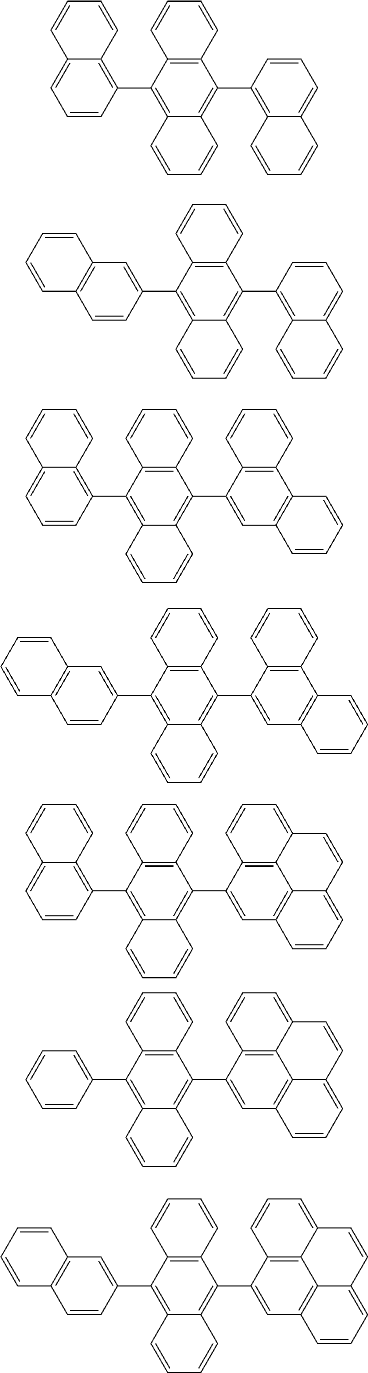

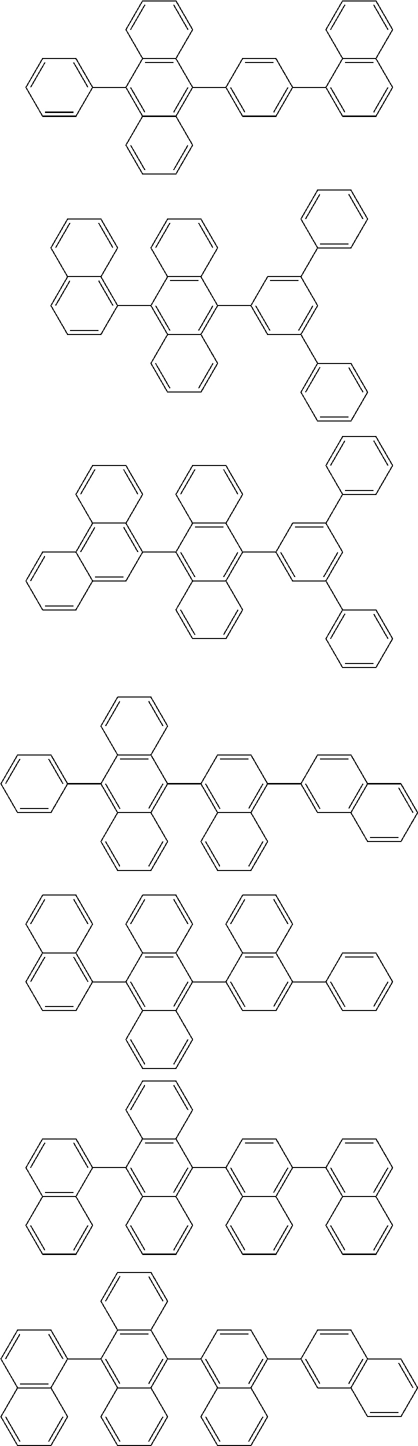

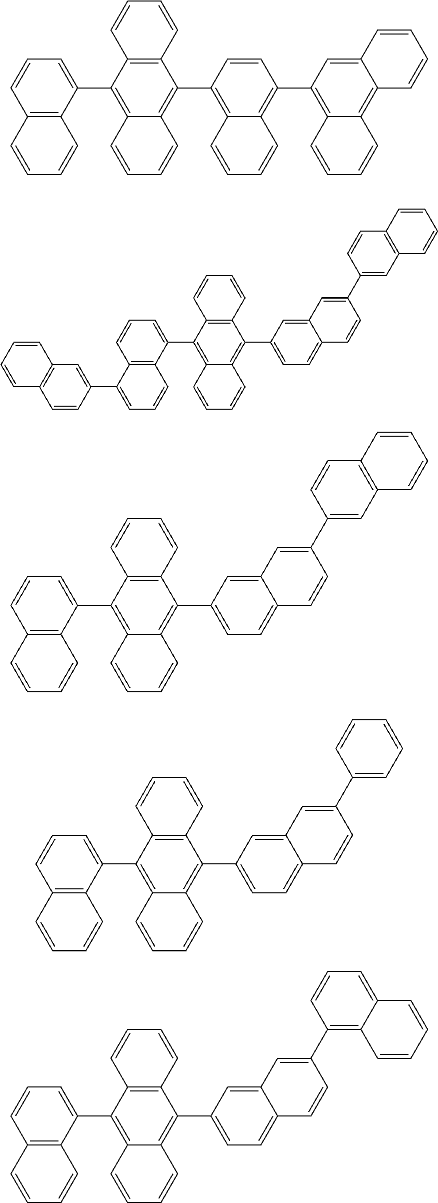

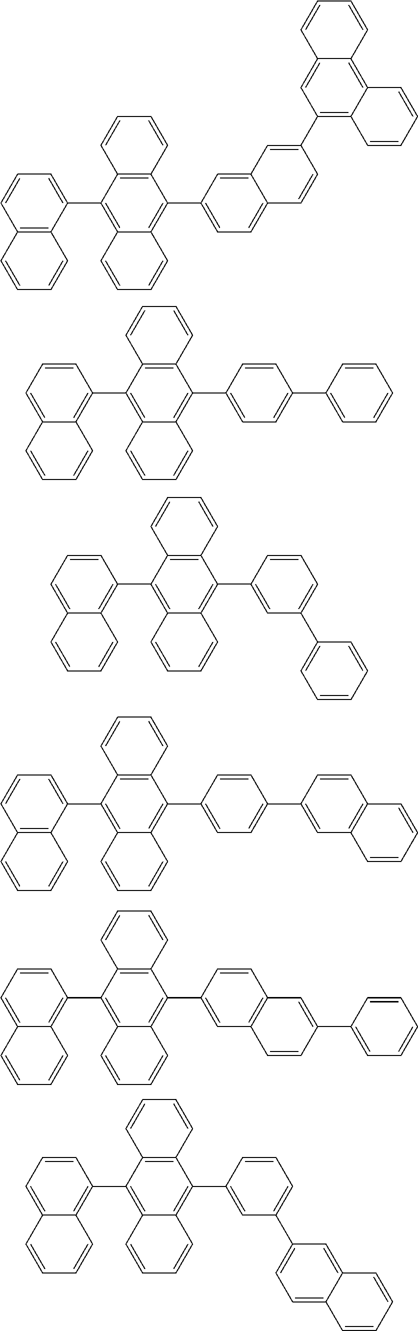

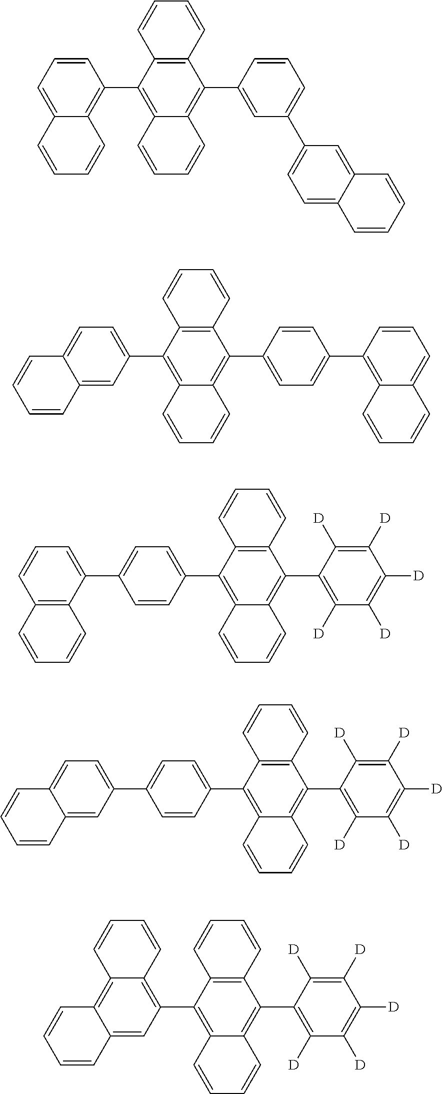

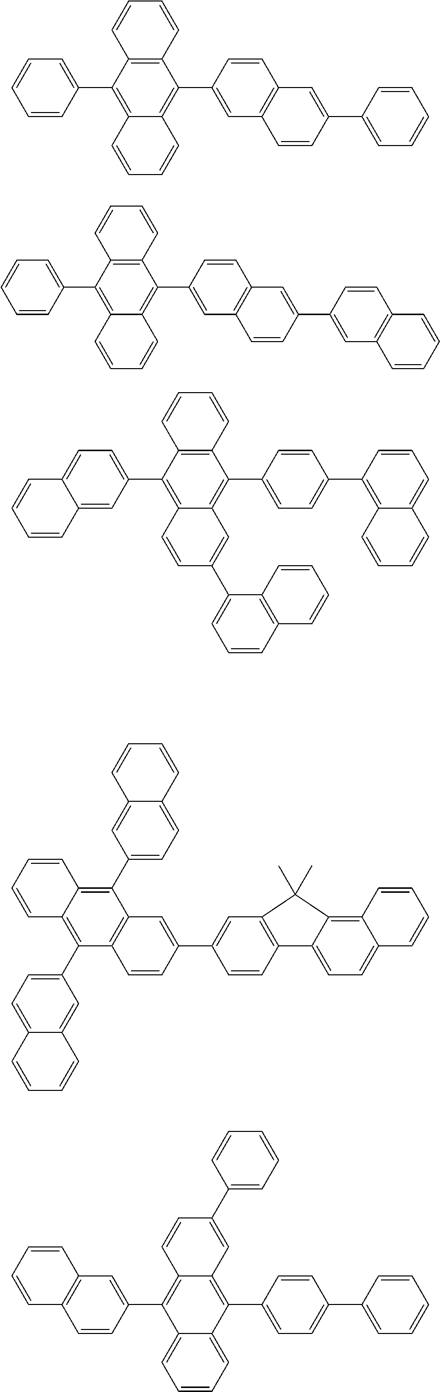

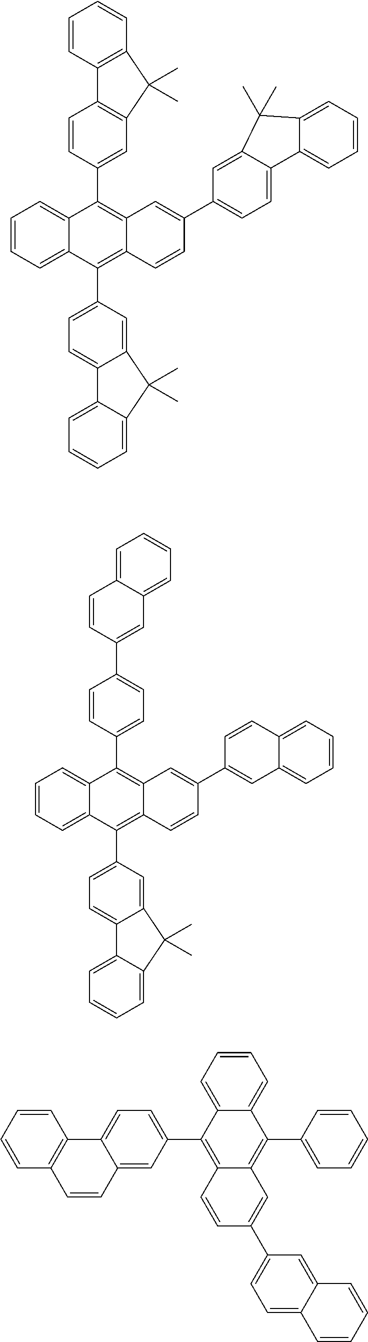

Also, an anthracene-based compound represented by Formula 401 below may be used as the host in the EML:

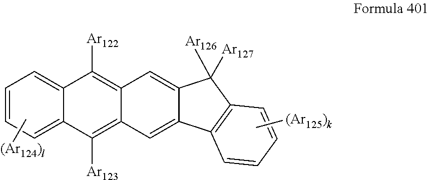

Wherein Ar₁₂₂ through Ar₁₂₅ may be each independently a substituted or unsubstituted C₁-C₁₀ alkyl group or a substituted or unsubstituted C₅-C₆₀ aryl group.

In Formula 401 above, Ar₁₂₆ and Ar₁₂₇ may be each independently a C₁-C₁₀ alkyl group (e.g., a methyl group, an ethyl group, or a propyl group).

In Formula 401 above, k and 1 may be each independently an integer of 0 to 4. For example, k and l may be each independently 0, 1, or 2.

For example, the anthrecene-based compound of Formula 401 may be, but is not limited to, one of the following compounds:

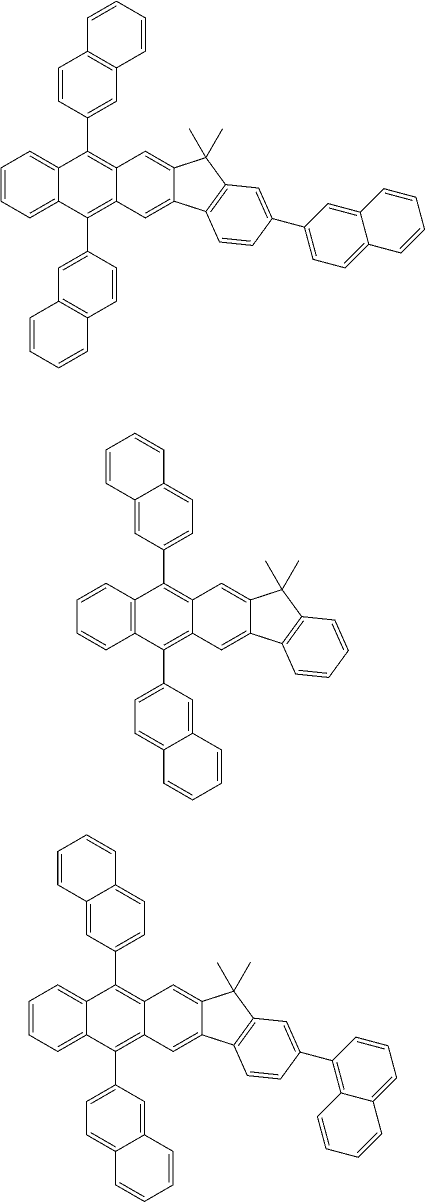

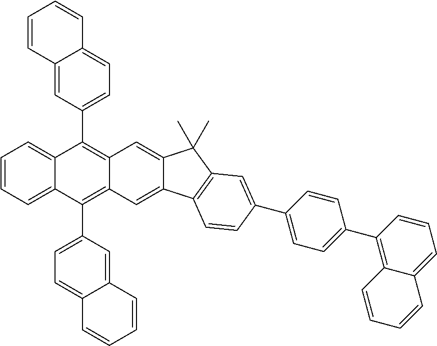 [SMILES: CC1(C)c2cc(-c3ccc(-c4cccc5ccccc45)cc3)ccc2-c2cc3c(-c4ccc5ccccc5c4)c4ccccc4c(-c4ccc5ccccc5c4)c3cc21]

If the OLED is a full-color OLED, the EML may be patterned as a red EML, a green EML, and a blue EML. In this regard, the above-described condensed-cyclic compounds may be included in the blue EML as a blue fluorescent dopant.

At least one of the red EML, the green EML, and the blue EML may include the following dopants.

For example, compounds described below may be used as blue dopants, but are not limited thereto. Herein, ppy=phenylpyridine.

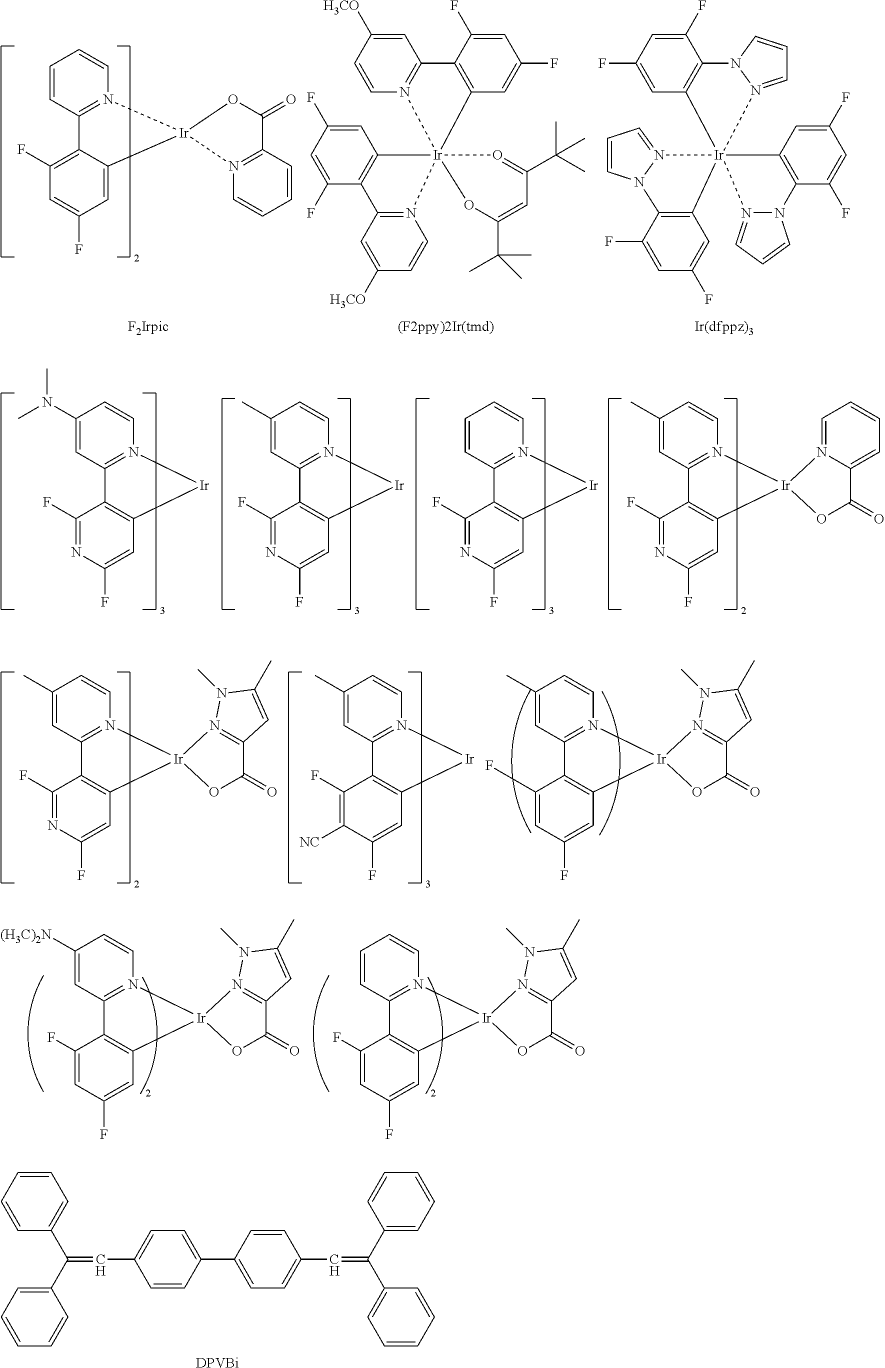

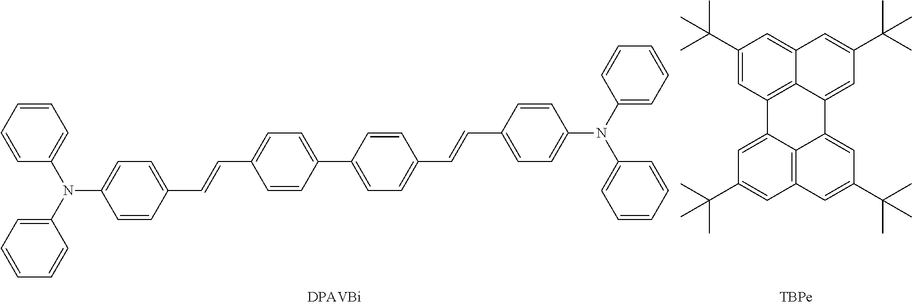

For example, compounds described below may be used as red dopants, but are not limited thereto.

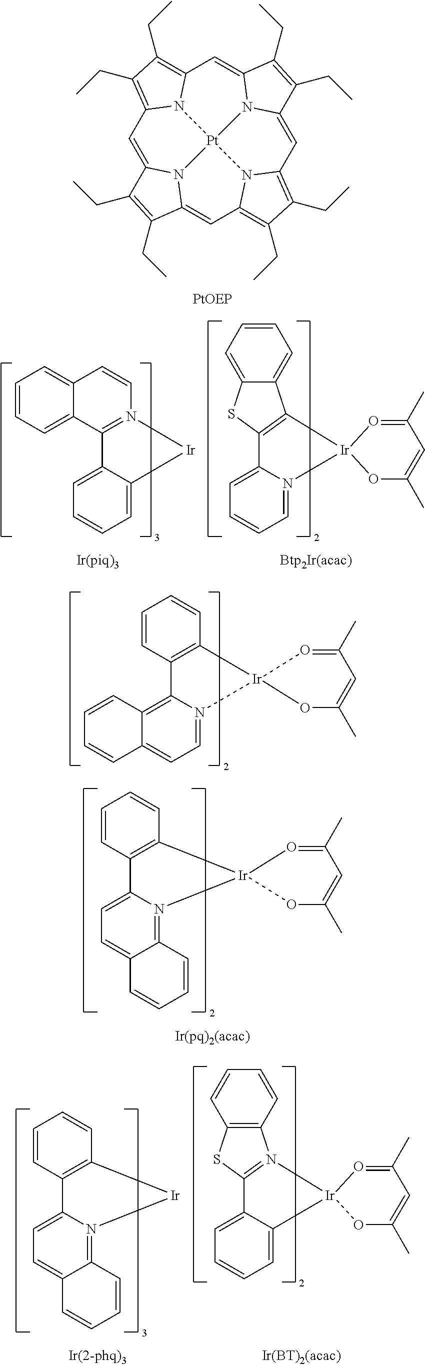

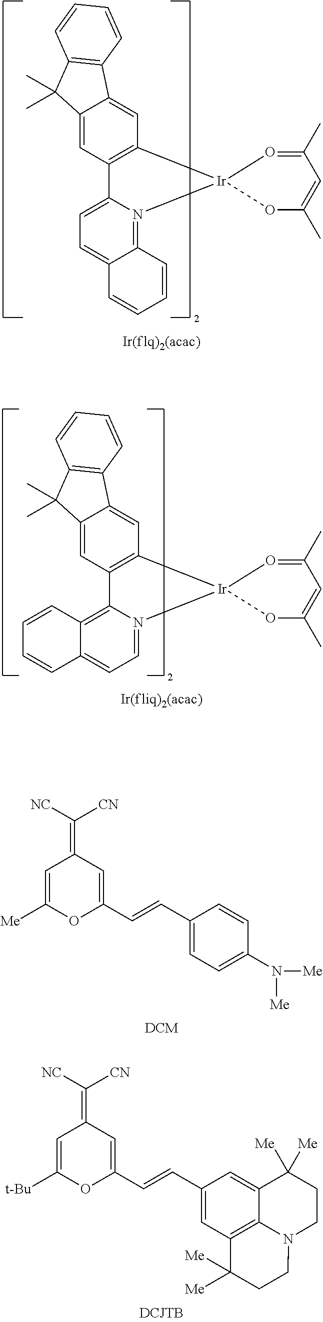

For example, compounds described below may be used as green dopants, but are not limited thereto.

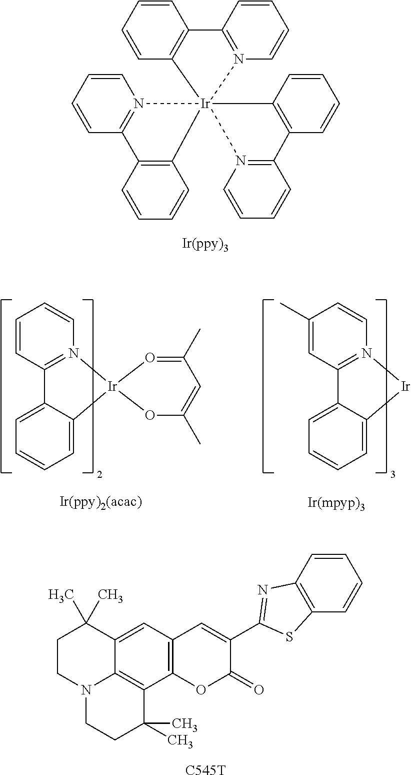

Examples of the dopant included in the EML include Pt-complexes below, but are not limited thereto:

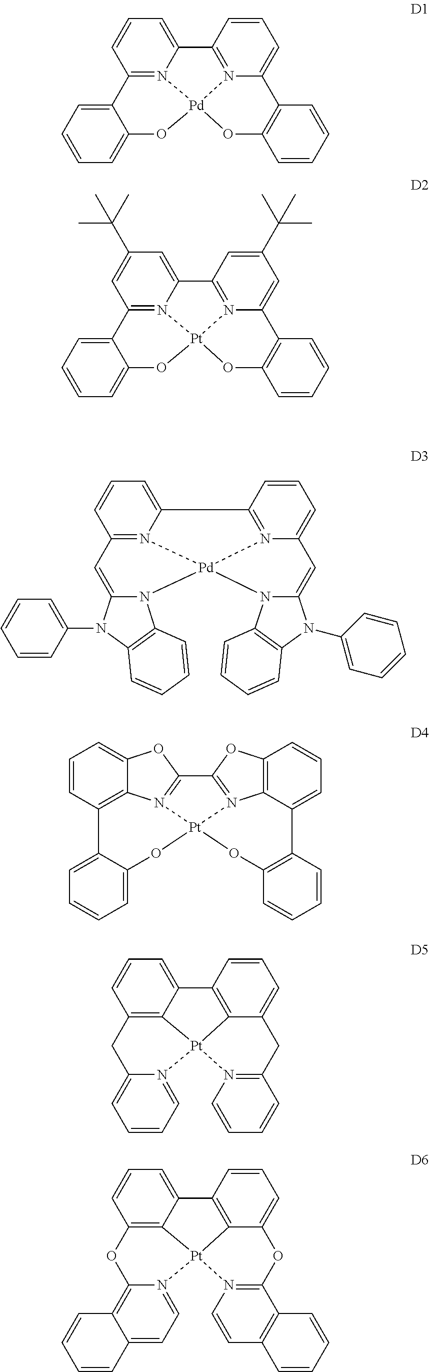

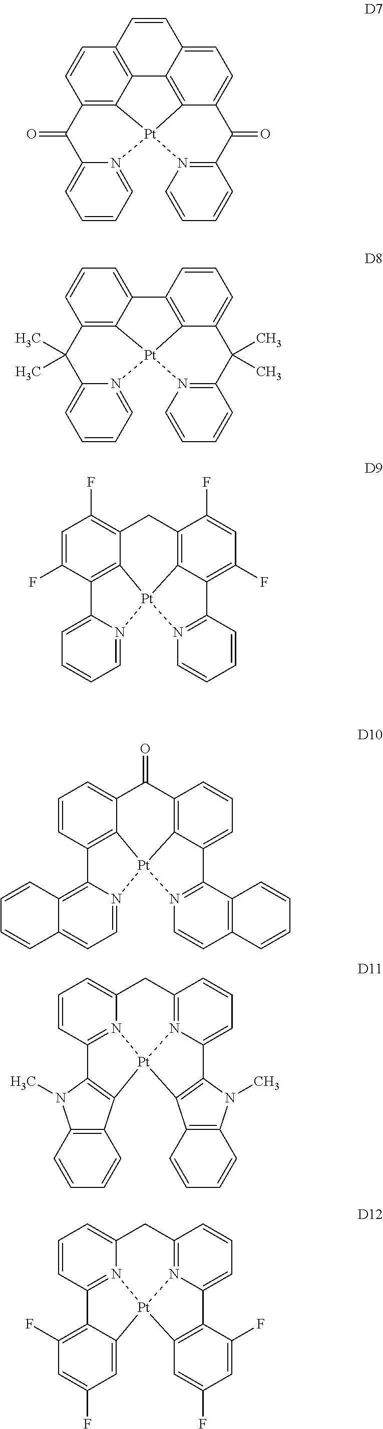

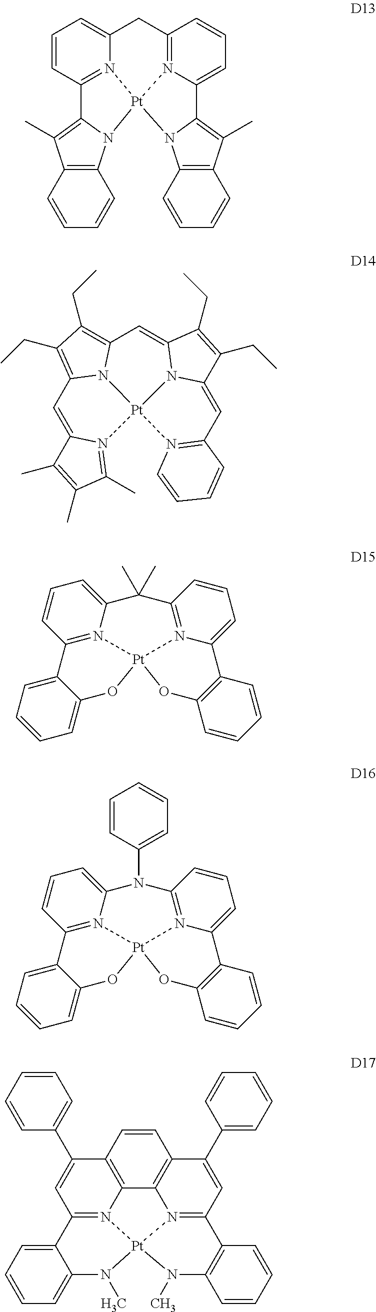

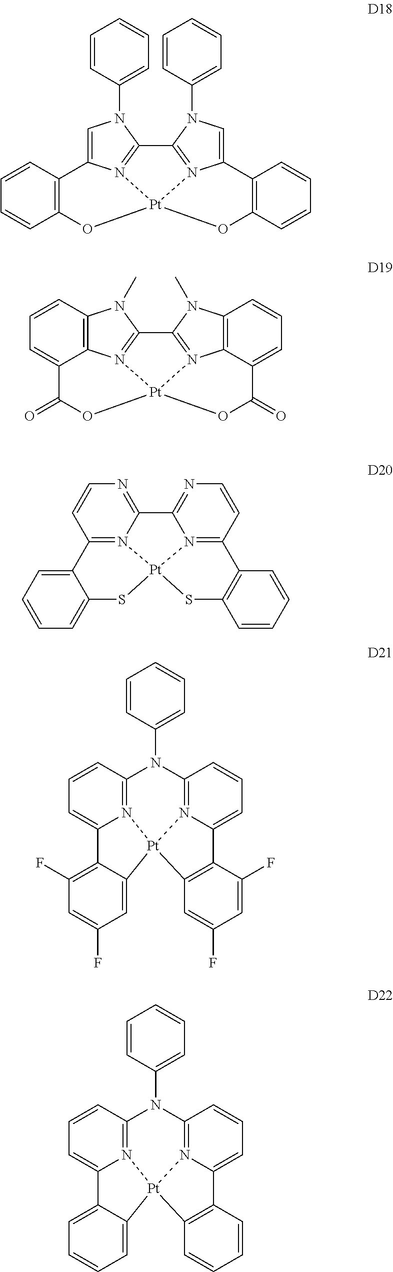

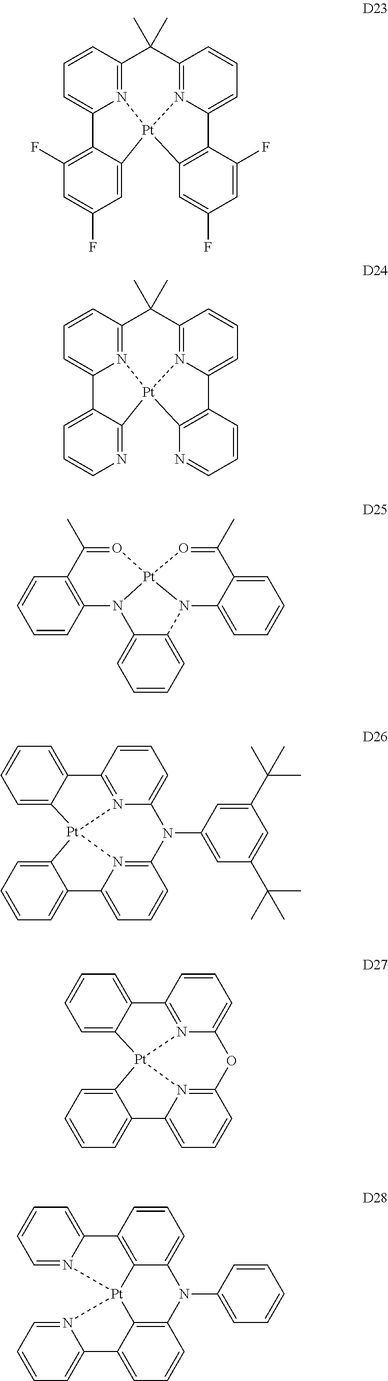

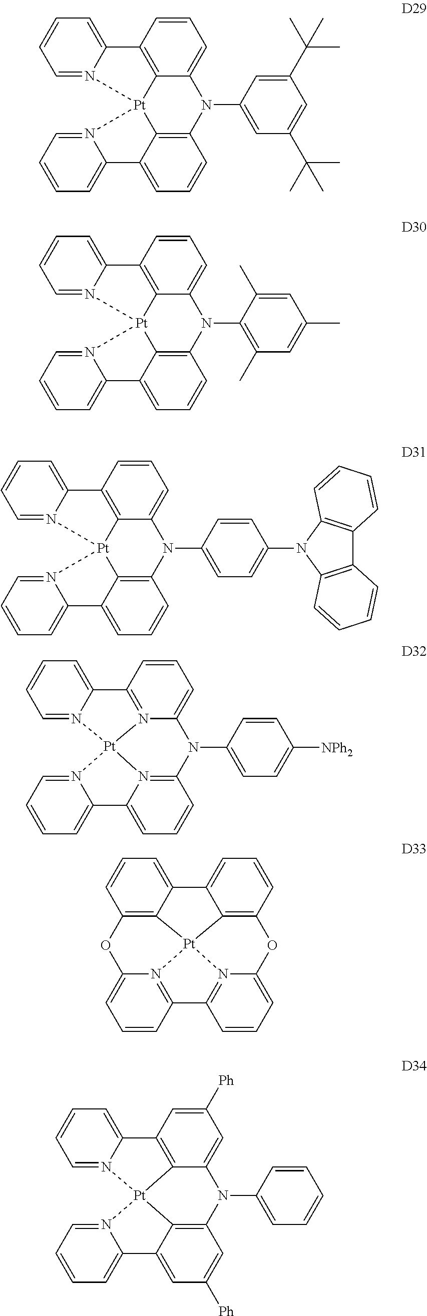

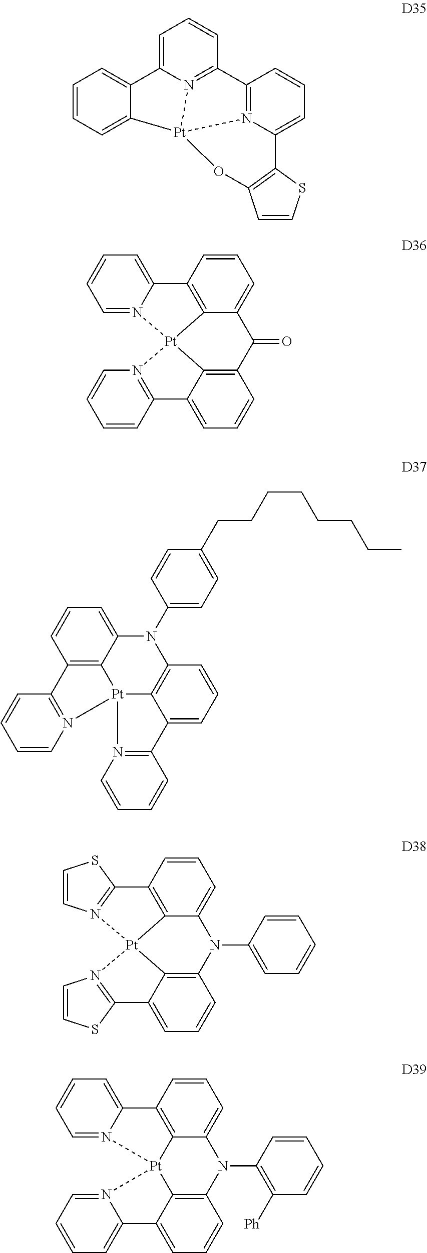

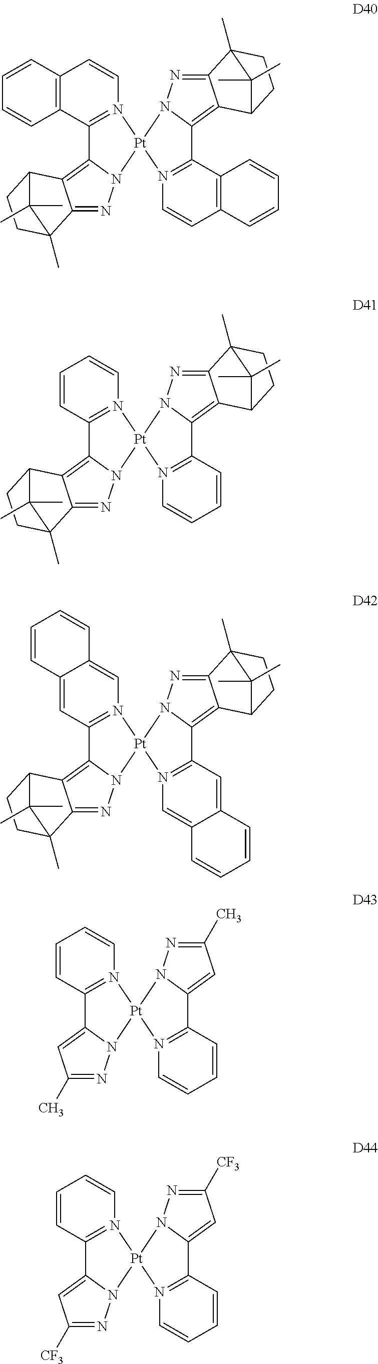

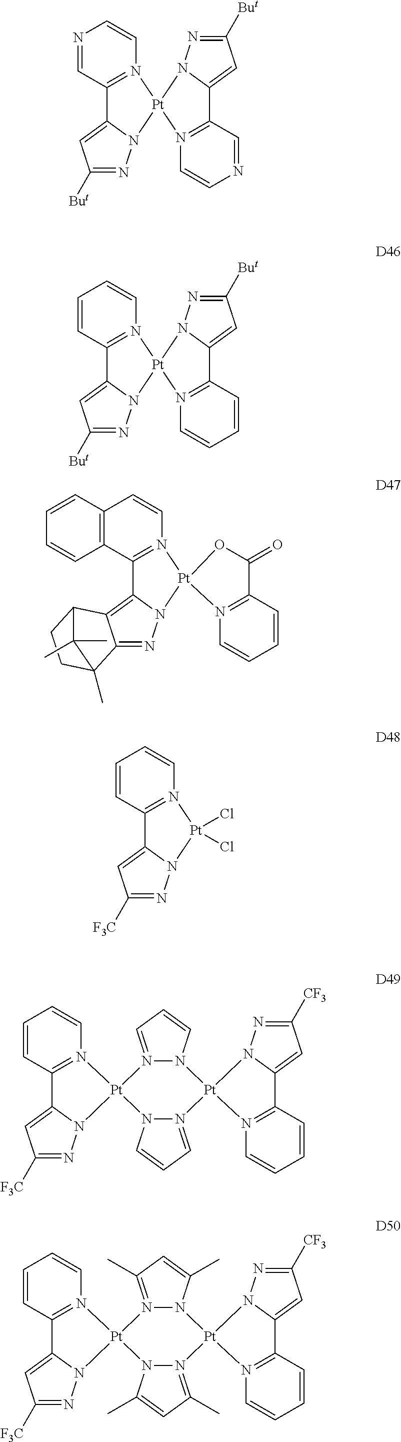

Also, examples of the dopant included in the EML may include, but are not limited to, Os-complexes:

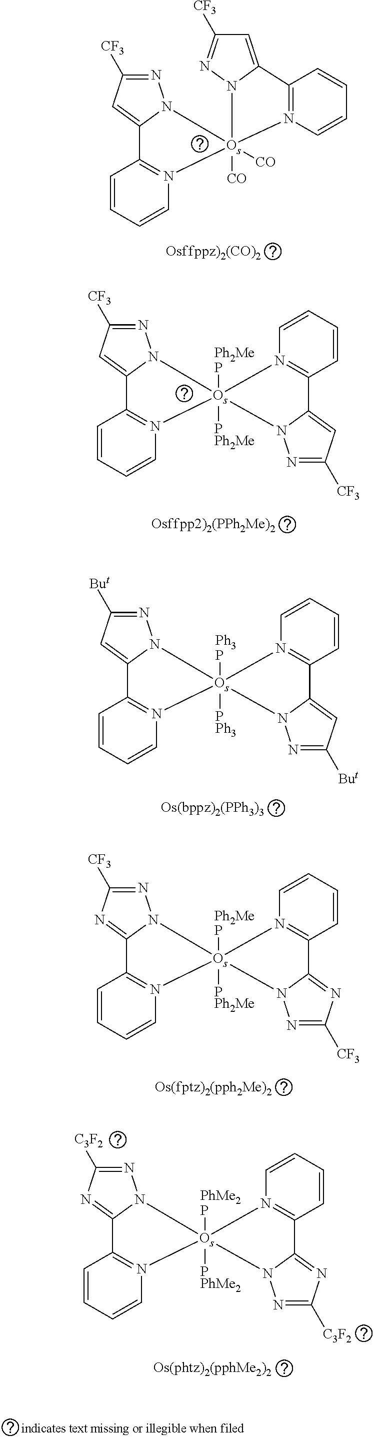

When the EML includes a host and a dopant, the amount of the dopant in the EML may be generally in the range of about 0.01 to about 15 parts by weight based on 100 parts by weight of the host, but is not limited thereto.

The thickness of the EML may be in the range of about 100 Å to about 1,000 Å. In some embodiments, the thickness of the EML may be in the range of about 200 Å to about 600 Å. When the thickness of the EML is within these ranges, excellent luminescent properties may be obtained without a substantial increase in driving voltage.

Next, an ETL may be formed using various methods such as vacuum deposition, spin coating, or casting. When the ETL is formed by vacuum deposition or spin coating, the deposition and coating conditions may vary according to a used compound. However, in general, the deposition and coating conditions may be almost the same as the condition for forming the HIL. A material for forming the ETL may be a known electron transporting material that stably transports electrons injected from a cathode. Examples of the known electron transporting material may include, but are not limited to, a quinoline derivative such as tris(8-quinolinolate)aluminum (Alq₃), TAZ, Balq, beryllium bis(benzoquinolin-10-olate (Bebq₂), ADN, and known materials such as Compound 201 and Compound 202 below.

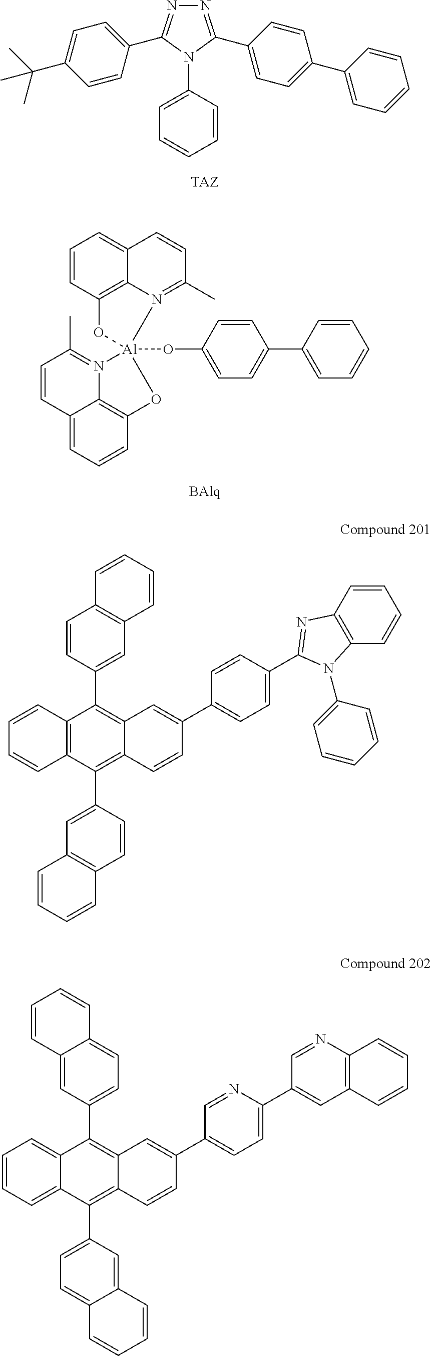

The thickness of the ETL may be in the range of about 100 Å to about 1,000 Å. In some embodiments, the thickness of the ETL may be in the range of about 150 Å to about 500 Å. When the thickness of the ETL is within these ranges, satisfactory electron transport properties may be obtained without a substantial increase in driving voltage.

In addition, the ETL may further include a metal-containing material, in addition to a known electron transporting organic compound.

The metal-containing material may include a Li-complex. Examples of the Li-complex may include lithium quinolate (LiQ) and Compound 203 below:

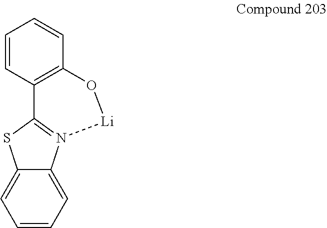

Also, an ELL, which facilitates electron injection from a cathode, may be formed on the ETL, and a material for forming the EIL is not particularly limited.

The material for forming the EIL may include a known material for forming an EIL, such as LiF, NaCl, CsF, Li₂O, or BaO. The deposition condition of the EIL may vary according a used compound. However, in general, the condition may be almost the same as the condition for forming the HIL.

The thickness of the EIL may be in the range of about 1 Å to about 100 Å. In some embosiments, the thickness of the EIL may be in the range of about 3 Å to about 90 Å. When the thickness of the EIL is within these ranges, satisfactory electron injection properties may be obtained without a substantial increase in driving voltage.

A second electrode 17 is formed on the organic layer 15. The second electrode 17 may be a cathode, which is an electron injection electrode. In this regard, a metal for forming the second electrode 17 may include a metal having low work function, such as metal, an alloy, an electric conducting compound, and mixtures thereof. In particular, the second electrode 17 may be formed as a thin film by using lithium (Li), magnesium (Mg), aluminum (Al), aluminum-lithium (Al—Li), calcium (Ca), magnesium-indium (Mg—In), or magnesium-silver (Mg—Ag), thus being transparent. In order to obtain a top-emission type organic light-emitting diode, the second electrode 17 may be formed as a transparent electrode by using ITO or IZO.

The OLED has been described with reference to FIG. 1, but is not limited thereto.

Also, when a phosphorescent dopant is included in the EML, a HBL may be formed between the ETL and the EML or between the H-functional layer and the EML by vacuum deposition, spin coating, casting or LB deposition so as to prevent triplet excitons or holes from being diffused to the ETL. When the HBL is formed by vacuum deposition or spin coating, the conditions thereof may vary according to a used compound. However, in general, the deposition and coating conditions may be almost the same as the condition for forming the HIL. The HBL may include a known hole blocking material. Examples of the known hole blocking material may include an oxadiazole deriative, a triazole derivative, and a phenanthroline derivative. For example, BCP may be used as a hole blocking material.

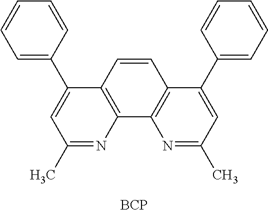 [SMILES: Cc1cc(-c2ccccc2)c2ccc3c(-c4ccccc4)cc(C)nc3c2n1 ; "BCP"]

The thickness of the HBL may be in the range of about 20 Å to about 1,000 Å. In some embodiments, the thickness of the HBL may be in the range of about 30 Å to about 300 Å. When the thickness of the HBL is within these ranges, excellent hole blocking properties may be obtained without a substantial increase in driving voltage.

An OLED according to an embodiment of the present invention will now be described in greate detail with reference to the following Examples. These Examples are for illustrative purposes only and are not intended to limit the scope of the invention.

### EXAMPLES

### Synthesis Example 1

### Synthesis of Compound 3

Compound 3 was synthesized according to Reaction Scheme 1 below:

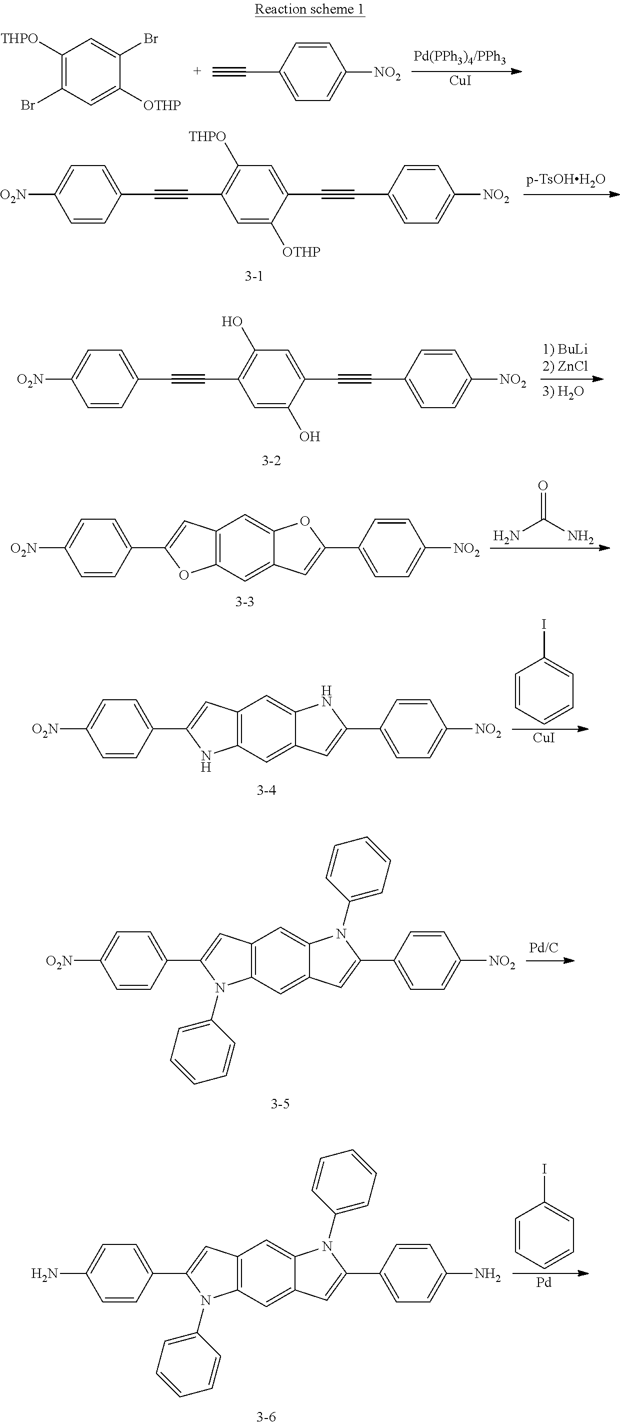

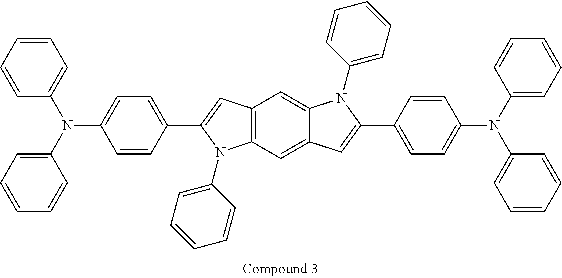 [SMILES: c1ccc(N(c2ccccc2)c2ccc(-c3cc4cc5c(cc(-c6ccc(N(c7ccccc7)c7ccccc7)cc6)n5-c5ccccc5)cc4n3-c3ccccc3)cc2)cc1]

### Synthesis of Intermediate 3-1

A pyperidine (30 ml)/triethylamine (90 ml) solution was maintained in a nitrogen atmosphere. To the solution 8.93 g (20 mmol) of 2-[2,5,dibromo-4-(tetrahydro-2H-2-pyranyloxy)phenoxy]tetrahydro-2H-pyran), 152 mg (0.8 mmol) of Cul, 209 mg (0.8 mmol) of triphenylphosphine, 462 mg (0.4 mmol) of tetrakistriphenylphosphinpalladium (Pd(PPh₃)₄), and 7.06 g (47.9 mmol) of 1-ethynyl-4-nitrobenzene were added and the mixed solution was stirred at 80° C. for 22 hours. The obtained reaction solution was cooled down to room temperature and then extracted three times with 100 ml of water and 100 ml of methylenechloride. The obtained organic layer was dried with magnesium sulfate and a solvent was evaporated therefrom to obtain a crude product. The crude product was purified with silicagel column chromatography to obtain 9.89 g of Intermediate 3-1 (yield 87%). The obtained compound was confirmed by mass spectrometry/fast atom bombardment (MS/FAB).

C₃₂H₂₈N₂O₈: calc. 568.18. found 568.25

### Synthesis of Intermediate 3-2

9.89 g (17.4 mmol) of Intermediate 3-1 was dissolved in 100 ml of methylenechloride and 100 ml of methanol, 330 mg (1.74 mmol) of p-toluenesulfonic acid monohydride (p-TsOH.H₂O) was added to the mixture, and the resulting mixture was stirred at room temperature for 4 hours. A solvent was evaporated therefrom to obtain a crude product. The crude product was recrystallized with acetone and hexane to obtain 6.40 g of Intermediate 3-2 (yield 92%). The obtained compound was confirmed by MS/FAB.

C₂₂H₁₂N₂O₆: calc. 400.34. found 400.40

### Synthesis of Intermediate 3-3

6.40 g (16.0 mmol) of Intermediate 3-2 was dissolved in 16 ml of tetrahydrofuran, 20 ml (32.0 mmol) of an n-BuLi solution (in hexane, 1.6 M) was slowly added to the mixed solution at 0° C., and the resulting solution was stirred at room temperature for 30 minutes. Subsequently, 32 ml of ZnCl₂ (in tetrahydrofuran, 1.0 M) was added to the obtained solution and the resultant solution was stirred at 120° C. for 3 hours. Water was then added thererto and the resultant mixture was stirred for 30 hours to obtain a solid. The solid was filtered with methanol and hexane. The obtained filtrate was recrystallized with ethylacetate and hexane to obtain 5.37 g of Intermediate 3-3 (yield 84%). The obtained product was confirmed by MS/FAB.

C₂₂H₁₂N₂O₆: calc. 400.34. found 400.42

### Synthesis of Intermediate 3-4

5.37 g (13.4 mmol) of Intermediate 3-3 was dissolved in 20 ml of toluene, 2.01 g (33.5 mmol) of urea was added thereto, and the resultant solution was stirred at 90° C. for 24 hours. The obtained reaction solution was cooled down to room temperature and then extracted three times with 100 ml of water and 100 ml of ethylacetate. The obtained organic layer was dried with magnesium sulfate and a solvent was evaporated therefrom to obtain a crude product. The crude product was purified with silicagel column chromatography to obtain 2.99 g of Intermediate 3-4 (yield 56%). The obtained compound was confirmed by MS/FAB.

C₂₂H₁₄N₄O₄: calc. 398.10. found 398.15

### Synthesis of Intermediate 3-5

2.99 g (7.50 mmol) of Intermediate 3-4, 2.30 g (11.3 mmol) of iodobenzene, 0.14 g (0.75 mmol) of Cul, 0.03 g (0.15 mmol) of 18-crown-6, and 3.13 g (22.5 mmol) of K₂CO₃ were dissolved in 30 ml of 1,3-dimethyl-3,4,5,6-tetrahydro-2(1H)-pyrimidinone (DMPU) and the mixed solution was stirred at 170° C. for 12 hours. The obtained reaction solution was cooled down to room temperature and then extracted three times with 50 ml of water and 50 ml of dichloromethane. The obtained organic layer was dried with magnesium sulfate and a solvent was evaporated therefrom to obtain a crude product. The crude product was purified with silicagel column chromatography to obtain 3.79 g of Intermediate 3-5 (yield 92%). The obtained compound was confirmed by MS/FAB.

C₃₄H₂₂N₄O₄: calc. 550.16. found 550.23

### Synthesis of Intermediate 3-6

3.79 g (6.88 mmol) of Intermediate 3-5 was dissolved in 50 ml of methanol, and 240 mg (0.68 mmol) of palladium on carbon (30% wt.) was slowly added to the mixed solution. After a hydrogen balloon was installed, the resultant solution was stirred at room temperature for 12 hours. After the reaction was terminated, the stirred solution was filtered with a silicagel-accumulated column and a solvent was evaporated therefrom to obtain a crude product. The crude product was purified with silicagel column chromatography to obtain 2.96 g of Intermediate 3-6 (yield 88%). The obtained compound was confirmed by MS/FAB.

C₃₄H₂₆N₄: calc. 490.21. found 490.25

### Synthesis of Compound 3

2.96 g (6.03 mmol) of Intermediate 3-6, 6.18 g (30.0 mmol) of iodobenzene, 440 mg (0.48 mmol) of Pd₂(dba)₃, 970 mg (0.48 mmol) of tri-tert-butylphosphine (P(t-Bu)₃), and 5.40 g (48.2 mmol) of KOtBu were dissolved in 150 ml of toluene, and the mixed solution was stirred at 85° C. for 4 hours. The obtained reaction solution was cooled down to room temperature and then extracted three times with 100 ml of water and 100 ml of diethylether. The obtained organic layer was dried with magnesium sulfate and a solvent was evaporated therefrom to obtain a crude product. The crude product was purified with silicagel column chromatography to obtain 3.64 g of Compound 3 (yield 76%). The obtained compound was confirmed by MS/FAB and ¹H nuclear magnetic resonance (NMR).

C₅₈H₄₂N₄: calc. 794.34. found 794.40

¹H NMR (CDCl₃, 400 MHz) δ (ppm) 7.84-7.83 (m, 2H), 7.43-7.31 (m, 13H), 7.08-7.03 (m, 8H), 6.67-6.63 (m, 5H), 6.58-6.57 (m, 2H), 6.52-6.50 (m, 4H), 6.17-6.13 (m, 8H)

### Synthesis Example 2

### Synthesis of Compound 19

### Synthesis of Intermediate 19-7

Intermediate 19-7 was prepared in the same manner as in the synthesis of Compoudn 3, except that 12.00 mmol of Intermediate 3-6 was used and 4-bromo-biphenyl was used instead of iodobenzene. The obtained compound was confirmed by MS/FAB.

C₅₈H₄₂N₄: calc. 794.34. found 794.39

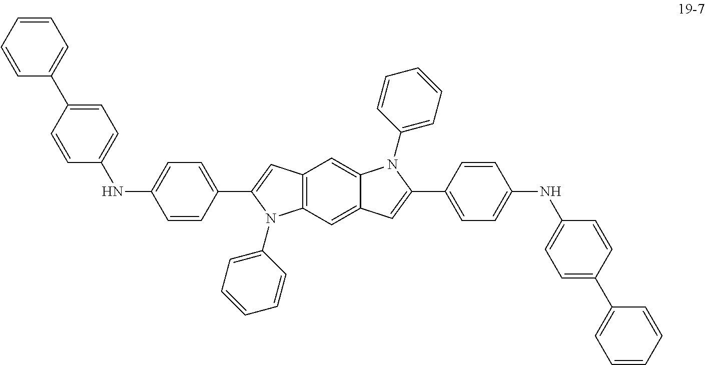 [SMILES: c1ccc(-c2ccc(Nc3ccc(-c4cc5cc6c(cc(-c7ccc(Nc8ccc(-c9ccccc9)cc8)cc7)n6-c6ccccc6)cc5n4-c4ccccc4)cc3)cc2)cc1]

### Synthesis of Compound 19

Compound 19 was synthesized in the same manner as in the synthesis of Compoudn 3, except that Intermediate 19-7 was used instead of Intermediate 3-6. The obtained compound was confirmed by MS/FAB and ¹H NMR.

C₇₀H₅₀N₄: calc. 946.40. found 946.45

¹H NMR (CDCl₃, 400 MHz) δ (ppm) 7.84-7.83 (m, 2H), 7.64-7.61 (m, 4H), 7.52-7.39 (m, 4H), 7.45-7.31 (m, 20H), 7.08-7.04 (m, 4H), 6.86-6.82 (m, 4H), 6.66-6.57 (m, 8H), 6.23-6.20 (m, 4H)

### Synthesis Example 3

### Synthesis of Compound 41

Compound 41 was synthesized according to Reaction Scheme 2 below:

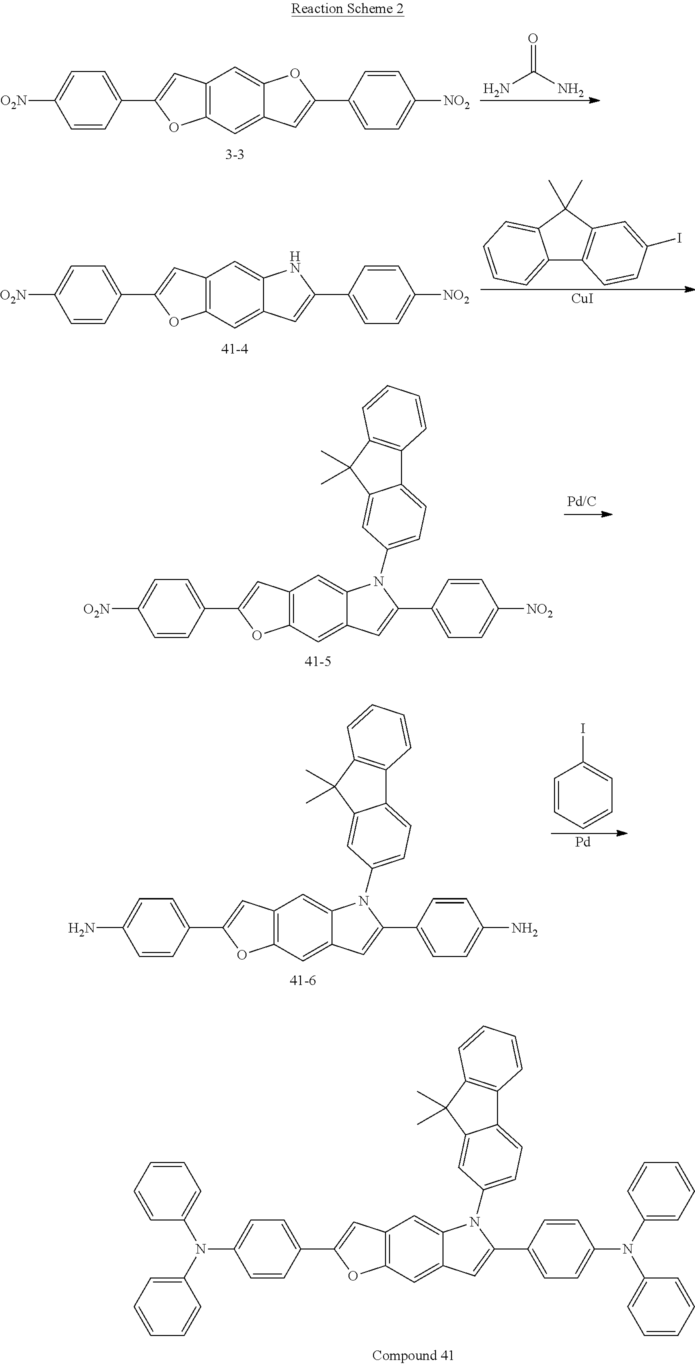

### Synthesis of Intermediate 41-4

4.00 g (10.0 mmol) of Intermediate 3-3 was dissolved in 20 ml of toluene, 600 mg (10.0 mmol) of urea was added thereto, and the resultant solution was stirred at 90° C. for 24 hours. The obtained reaction solution was cooled down to room temperature and then extracted three times with 100 ml of water and 100 ml of ethylacetate. The obtained organic layer was dried with magnesium sulfate and a solvent was evaporated therefrom to obtain a crude product. The crude product was purified with silicagel column chromatography to obtain 2.96 g of Intermediate 41-4 (yield 74%). The obtained compound was confirmed by MS/FAB.

C₂₂H₁₃N₃O₅: calc. 399.08. found 399.13

### Synthesis of Intermediate 41-5

Intermediate 41-5 was prepared in the same manner as in the synthesis of Intermediate 3-5, except that Intermediate 41-4 was used instead of Intermediate 3-4 and 9,9-dimethyl-2-iodofluorene was used instead of iodobenzene. The obtained compound was confirmed by MS/FAB.

C₃₇H₂₅N₃O₅: calc. 591.17. found 591.24

### Synthesis of Intermediate 41-6

Intermediate 41-6 was synthesized in the same manner as in the synthesis of Intermediate 3-6, except that Intermediate 41-5 was used instead of Intermediate 3-5. The obtained compound was confirmed by MS/FAB.

C₃₇H₂₉N₃O: calc. 531.23. found 531.29

### Synthesis of Compound 41

Intermediate 41-6 was synthesized in the same method as in the synthesis of Compound 3, except that Intermediate 41-6 was used instead of Intermediate 3-6. The obtained compound was confirmed by MS/FAB and ¹H NMR.

C₆₁H₄₅N₃O: calc. 835.35. found 835.41

¹H NMR (CDCl₃, 400 MHz) δ (ppm) 7.87-7.85 (m, 1H), 7.72-7.71 (m, 1H), 7.65-7.60 (m, 4H), 7.37-7.31 (m, 4H), 7.21-7.19 (m, 1H), 7.13-7.01 (m, 10H), 6.88-6.87 (m, 1H), 6.66-6.63 (m, 4H), 6.58-6.57 (m, 1H), 6.52-6.48 (m, 4H), 6.16-6.12 (m, 8H), 1.64 (s, 6H)

### Synthesis Example 4

### Synthesis of Compound 61

Compound 61 was synthesized according to Reaction Scheme 3 below:

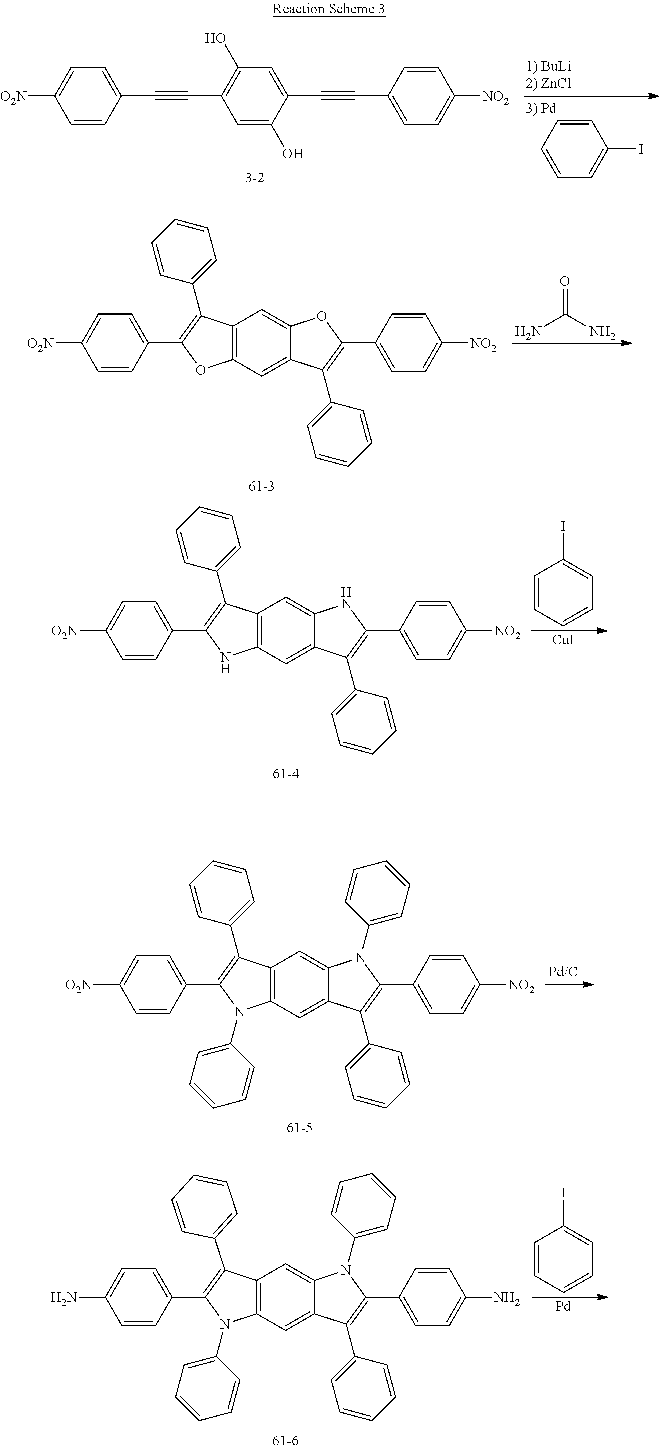

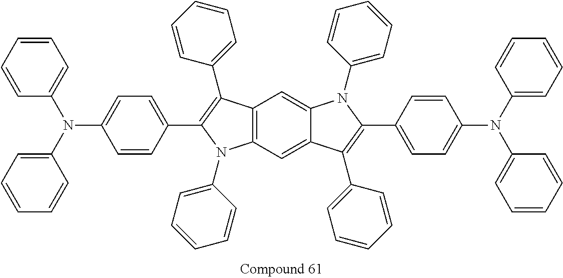 [SMILES: c1ccc(-c2c(-c3ccc(N(c4ccccc4)c4ccccc4)cc3)n(-c3ccccc3)c3cc4c(-c5ccccc5)c(-c5ccc(N(c6ccccc6)c6ccccc6)cc5)n(-c5ccccc5)c4cc23)cc1]

### Synthesis of Intermediate 61-3

6.40 g (16.0 mmol) of Intermediate 3-2 was dissolved in 16 ml of tetrahydrofuran, 20 ml (32.0 mmol) of an n-BuLi solution (in hexane, 1.6 M) was slowly added to the mixed solution at 0° C., and the resulting solution was stirred at room temperature for 30 minutes. Subsequently, 32 ml of ZnCl₂ (in tetrahydrofuran, 1.0 M) was added to the obtained solution and the resultant solution was stirred at 120° C. for 3 hours. The obtained reaction solution was cooled down to room temperature, and to the reaction solution were added 1.46 g (1.60 mmol) of Pd₂(dba)₃, 1.29 g (6.4 mmol) of tri-tert-butylphosphine (P(t-Bu)₃), 16 ml of 1-methyl-2-pyrrolidinone (NMP), and 7.83 g (38.4 mmol) of iodobenzene. The resultant solution was then stirred at 100° C. for 15 hours. After being cooled down to room temperature, the reaction solution was filtered and then washed several times with methanol and ethylacetate. The resultant product was recrystallized with ethylacetate and hexane to obtain 5.74 g of Intermediate 61-3 (yield 65%). The obtained compound was confirmed by MS/FAB.

C₃₄H₂₀N₂O₆: calc. 552.13. found 552.20

### Synthesis of Intermediate 61-4

Intermediate 61-4 was synthesized in the same manner as in the synthesis of Intermediate 3-4, except that Intermediate 61-3 was used instead of Intermediate 3-3. The obtained compound was confirmed by MS/FAB.

C₃₄H₂₄N₄O₄: calc. 550.16. found 550.22

### Synthesis of Intermediate 61-5

Intermediate 61-5 was synthesized in the same manner as in the synthesis of Intermediate 3-5, except that Intermediate 61-4 was used instead of Intermediate 3-4. The obtained compound was confirmed by MS/FAB.

C₄₆H₃₀N₄O₄: calc. 702.22. found 702.26

### Synthesis of Intermediate 61-6

Intermediate 61-6 was synthesized in the same manner as in the synthesis of Intermediate 3-6, except that Intermediate 61-5 was used instead of Intermediate 3-5. The obtained compound was confirmed by MS/FAB.

C₄₆H₃₄N₄: calc. 642.27. found 642.33

### Synthesis of Compound 61

Compound 61 was synthesized in the same manner as in the synthesis of Compound 3, except that Intermediate 61-6 was used instead of Intermediate 3-6. The obtained compound was confirmed by MS/FAB and ¹H NMR.

C₇₀H₅₀N₄: calc. 946.40. found 946.45

¹H NMR (CDCl₃, 400 MHz) δ (ppm) 8.17-8.16 (m, 2H), 7.49-7.33 (m, 20H), 7.08-7.03 (m, 8H), 6.94-6.91 (m, 4H), 6.66-6.63 (m, 4H), 6.28-6.25 (m, 4H), 6.16-6.13 (m, 8H)

### Synthesis Example 5

### Synthesis of Compound 64

Compound 64 was synthesized according to Reaction Scheme 4 below:

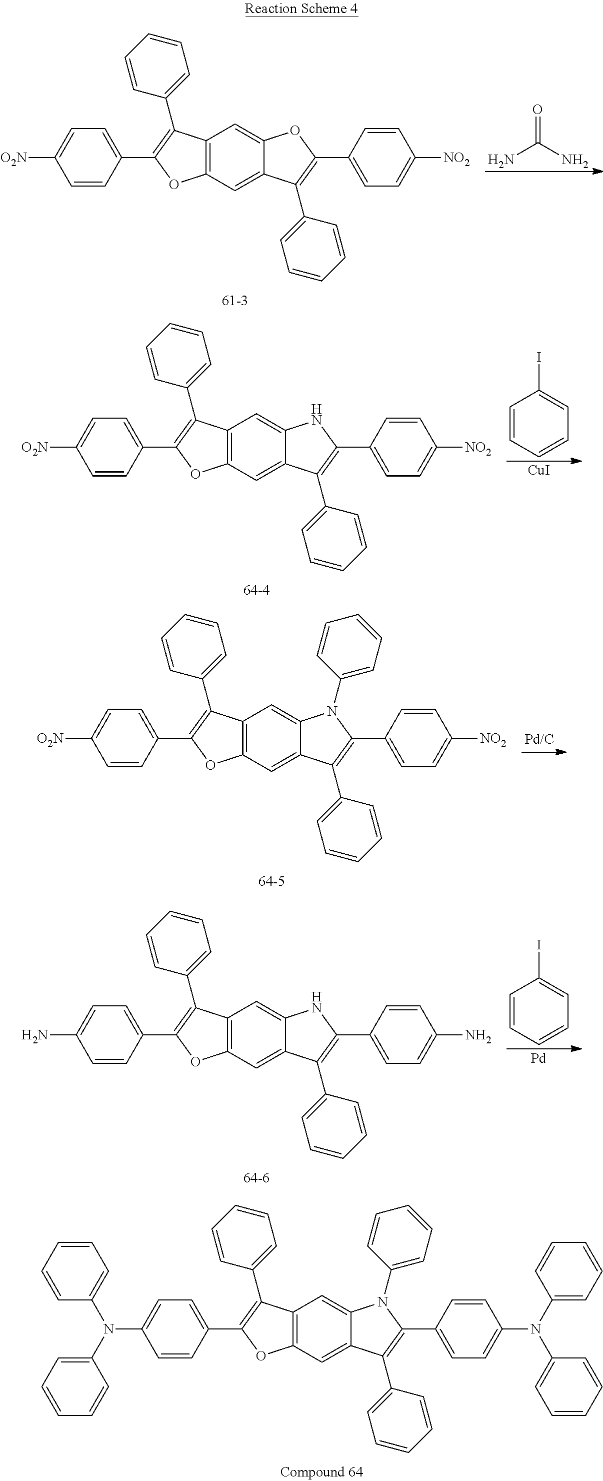

### Synthesis of Intermediate 64-4

Intermediate 64-4 was synthesized in the same manner as in the synthesis of Intermediate 41-4, except that Intermediate 61-3 was used instead of Intermediate 3-3. The obtained compound was confirmed by MS/FAB.

C₃₄H₂₁N₃O₅: calc. 551.14. found 551.20

### Synthesis of Intermediate 64-5

Intermediate 64-5 was synthesized in the same manner as in the synthesis of Intermediate 3-5, except that Intermediate 64-4 was used instead of Intermediate 3-4. The obtained compound was confirmed by MS/FAB.

C₄₀H₂₅N₃O₅: calc. 627.17. found 627.23

### Synthesis of Intermediate 64-6

Intermediate 64-6 was synthesized in the same manner as in the synthesis of Intermediate 3-6, except that Intermediate 64-5 was used instead of Intermediate 3-5. The obtained compound was confirmed by MS/FAB.

C₄₀H₂₉N₃O: calc. 567.23. found 567.30

### Synthesis of Compound 64

Compound 64 was synthesized in the same manner as in the synthesis of Compound 64, except that Intermediate 64-6 was used instead of Intermediate 3-6. The obtained compound was confirmed by MS/FAB and ¹H NMR.

C₆₄H₄₅N₃O: calc. 871.35. found 871.40

¹H NMR (CDCl₃, 400 MHz) δ (ppm) 7.92 (s, 1H), 7.91 (s, 1H), 7.64-7.58 (m, 2H), 7.51-7.33 (m, 15H), 7.08-7.01 (m, 10H), 6.66-6.63 (m, 4H), 6.47-6.44 (m, 2H), 6.29-6.26 (m, 2H), 6.16-6.13 (m, 8H)

### Synthesis Example 6

### Synthesis of Compound 2

Compound 2 was synthesized in the same manner as in Synthesis Example 1, except that 2-iodo-2-methylpropane was used instead of iodobenzene in the synthesis of Intermediate 3-5. The obtained compound was confirmed by MS/FAB and ¹H NMR.

C₅₄H₅₀N₄: calc. 754.40. found 754.45

¹H NMR (CDCl₃, 400 MHz) δ (ppm) 7.84-7.83 (m, 2H), 7.31-7.28 (m, 4H), 7.08-7.03 (m, 8H), 6.67-6.63 (m, 4H), 6.44-6.40 (m, 4H), 6.34-6.33 (m, 2H), 6.16-6.13 (m, 8H), 1.78 (s, 18H)

### Synthesis Example 7

### Synthesis of Compound 6

Compound 6 was synthesized in the same manner as in Synthesis Example 1, except that 4-bromobiphenyl was used instead of iodobenzene in the synthesis of Intermediate 3-5. The obtained compound was confirmed by MS/FAB and ¹H NMR.

C₇₀H₅₀N₄: calc. 946.40. found 946.44

¹H NMR (CDCl₃, 400 MHz) δ (ppm) 7.87-7.86 (m, 2H), 7.63-7.60 (m, 4H), 7.53-7.45 (m, 9H), 7.42-7.29 (m, 10H), 7.08-7.04 (m, 8H), 6.66-6.63 (m, 4H), 6.56-6.50 (m, 5H), 6.17-6.13 (m, 8H)

### Synthesis Example 8

### Synthesis of Compound 8

Compound 8 was synthesized in the same manner as in Synthesis Example 1, except that bromobenzene-d₅ was used instead of iodobenzene in the synthesis of Intermediate 3-5. The obtained compound was confirmed by MS/FAB and ¹H NMR.

C₅₈H₃₂D₁₀N₄: calc. 804.40. found 804.46

¹H NMR (CDCl₃, 400 MHz) δ (ppm) 7.84-7.83 (m, 2H), 7.36-7.33 (m, 4H), 7.08-7.03 (m, 8H), 6.67-6.63 (m, 4H), 6.58-6.57 (m, 2H), 6.53-6.50 (m, 4H), 6.17-6.13 (m, 8H)

### Synthesis Example 9

### Synthesis of Compound 11

Compound 11 was synthesized in the same manner as in Synthesis Example 1, except that 9,9-dimethyl-2-iodofluorene was used instead of iodobenzene in the synthesis of Intermediate 3-5. The obtained compound was confirmed by MS/FAB and ¹H NMR.

C₇₆H₅₈N₄: calc. 1026.46. found 1026.50

¹H NMR (CDCl₃, 400 MHz) δ (ppm) 7.93-7.92 (m, 2H), 7.87-7.85 (m, 2H), 7.63-7.61 (m, 2H), 7.37-7.31 (m, 7H), 7.21-7.19 (m, 2H), 7.13-7.01 (m, 12H), 6.87-6.86 (m, 2H), 6.66-6.63 (m, 4H), 6.57-6.56 (m, 2H), 6.53-6.50 (m, 4H), 6.16-6.13 (m, 7H), 1.68 (s, 12H)

### Synthesis Example 10

### Synthesis of Compound 12

Compound 12 was synthesized in the same manner as in Synthesis Example 1, except that 3-bromo-9-phenyl-9H-carbazole was used instead of iodobenzene in the synthesis of Intermediate 3-5. The obtained compound was confirmed by MS/FAB and ¹H NMR.

C₈₂H₅₆N₆: calc. 1124.45. found 1124.51

¹H NMR (CDCl₃, 400 MHz) δ (ppm) 8.37-8.32 (m, 3H), 7.94-7.93 (m, 2H), 7.87-7.86 (m, 2H), 7.54-7.48 (m, 7H), 7.44-7.27 (m, 13H), 7.08-7.01 (m, 9H), 6.66-6.64 (m, 4H), 6.55-6.52 (m, 8H), 6.17-6.13 (m, 8H)

### Synthesis Example 11

### Synthesis of Compound 13

Compound 13 was synthesized in the same manner as in Synthesis Example 1, except that 3-bromopyridine was used instead of iodobenzene in the synthesis of Intermediate 3-5. The obtained compound was confirmed by MS/FAB and ¹H NMR.

C₅₆H₄₀N₆: calc. 796.33. found 796.36

¹H NMR (CDCl₃, 400 MHz) δ (ppm) 8.52-8.48 (m, 4H), 7.88-7.87 (m, 2H), 7.86-7.83 (m, 2H), 7.42-7.38 (m, 6H), 7.08-7.04 (m, 8H), 6.67-6.62 (m, 6H), 6.53-6.50 (m, 4H), 6.17-6.13 (m, 8H)

### Synthesis Example 12

### Synthesis of Compound 14

Compound 14 was synthesized in the same manner as in Synthesis Example 1, except that 2-bromo-4,6-diphenyl-1,3,5-triazine was used instead of iodobenzene in the synthesis of Intermediate 3-5. The obtained compound was confirmed by MS/FAB and ¹H NMR.

C₇₆H₅₂N₁₀: calc. 1104.43. found 1104.48

¹H NMR (CDCl₃, 400 MHz) δ (ppm) 8.78-8.74 (m, 8H), 8.32-8.30 (m, 2H), 7.69-7.65 (m, 8H), 7.58-7.56 (m, 4H), 7.42-7.38 (m, 4H), 7.25-7.24 (m, 2H), 7.08-7.02 (m, 9H), 6.86-6.84 (m, 4H), 6.66-6.63 (m, 4H), 6.17-6.12 (m, 7H)

### Synthesis Example 13

### Synthesis of Compound 17

Compound 17 was synthesized in the same manner as in Synthesis Example 2, except that 1-bromonaphthalene was used instead of 4-bromo-biphenyl in the synthesis of Intermediate 19-7. The obtained compound was confirmed by MS/FAB and ¹H NMR.

C₆₆H₄₆N₄: calc. 894.37. found 894.42

¹H NMR (CDCl₃, 400 MHz) δ (ppm) 8.18-8.16 (m, 3H), 7.87-7.84 (m, 5H), 7.50-7.31 (m, 16H), 7.25-7.21 (m, 3H), 7.07-7.01 (m, 4H), 6.74-6.72 (m, 2H), 6.65-6.63 (m, 3H), 6.57-6.56 (m, 2H), 6.30-6.27 (m, 4H), 6.07-6.05 (m, 4H).

### Synthesis Example 14

### Synthesis of Compound 21

Compound 21 was synthesized in the same manner as in Synthesis Example 2, except that 9,9-dimethyl-2-iodofluorene was used instead of 4-bromo-biphenyl in the synthesis of Intermediate 19-7. The obtained compound was confirmed by MS/FAB and ¹H NMR.

C₇₀H₅₈N₄: calc. 1026.46. found 1026.50

¹H NMR (CDCl₃, 400 MHz) δ (ppm) 7.84-7.83 (m, 2H), 7.78-7.76 (m, 2H), 7.56-7.54 (m, 2H), 7.44-7.30 (m, 16H), 7.14-7.04 (m, 8H), 6.69-6.63 (m, 4H), 6.57-6.56 (m, 2H), 6.52-6.50 (m, 4H), 6.39-6.38 (m, 2H), 6.24-6.21 (m, 4H), 1.64 (s, 12H)

### Synthesis Example 15

### Synthesis of Compound 24

Compound 24 was synthesized in the same manner as in Synthesis Example 2, except that 4-bromobenzonitrile was used instead of 4-bromo-biphenyl in the synthesis of Intermediate 19-7. The obtained compound was confirmed by MS/FAB and ¹H NMR.

C₆₀H₄₀N₆: calc. 844.33. found 844.37.

¹H NMR (CDCl₃, 400 MHz) δ (ppm) 7.84-7.83 (m, 2H), 7.43-7.31 (m, 18H), 7.08-7.03 (m, 4H), 6.76-6.73 (m, 4H), 6.67-6.58 (m, 8H), 6.23-6.20 (m, 4H)

### Synthesis Example 16

### Synthesis of Compound 25

Compound 25 was synthesized in the same manner as in Synthesis Example 1, except that 1-iodo-3,5-dimethylbenzene was used instead of iodobenzene in the synthesis of Compound 3. The obtained compound was confirmed by MS/FAB and ¹H NMR.

C₆₆H₅₈N₄: calc. 906.46. found 906.51

¹H NMR (CDCl₃, 400 MHz) δ(ppm) 7.85-7.84 (m, 2H), 7.43-7.30 (m, 14H), 6.80-6.76 (m, 4H), 6.61-6.58 (m, 6H), 6.27-6.25 (m, 8H), 2.34-2.32 (m, 24H)

### Synthesis Example 17

### Synthesis of Compound 31

Compound 31 was synthesized in the same manner as in Synthesis Example 2, except that Intermediate 31-7-(1) below was used instead of 4-bromo-biphenyl in the synthesis of Intermediate 19-7. The obtained compound was confirmed by MS/FAB and ¹H NMR.

C₈₂H₅₆F₂N₄: calc. 1134.44. found 1134.47

¹H NMR (CDCl₃, 400 MHz) δ (ppm) 7.85-7.84 (m, 2H), 7.72-7.69 (m, 3H), 7.66-7.60 (m, 6H), 7.56-7.49 (m, 9H), 7.44-7.31 (m, 20H), 7.14-7.10 (m, 2H), 7.08-7.03 (m, 4H), 6.63-6.57 (m, 2H), 6.36-6.33 (m, 4H), 6.15-6.11 (m, 4H)

### Synthesis Example 18

### Synthesis of Compound 32

Compound 32 was synthesized in the same manner as in Synthesis Example 1, except that bromobenzene-d₅ was used instead of iodobenzene in the synthesis of Compound 3. The obtained compound was confirmed by MS/FAB and ¹H NMR.

C₅₉H₂₂D₂₀N₄: calc. 827.47. found 827.52

¹H NMR (CDCl₃, 400 MHz) δ (ppm) 7.85-7.84 (m, 2H), 7.43-7.31 (m, 14H), 6.58-6.57 (m, 2H), 6.52-6.50 (m, 2H), 6.35-6.32 (m, 2H)

### Synthesis Example 19

### Synthesis of Compound 37

Compound 37 was synthesized in the same manner as in Synthesis Example 3, except that iodobenzene was used instead of 9,9-dimethyl-2-iodofluorene in the synthesis of Intermediate 41-5. The obtained compound was confirmed by MS/FAB and ¹H NMR.

C₅₂H₃₇N₃O: calc. 719.29. found 719.34

¹H NMR (CDCl₃, 400 MHz) δ (ppm) 7.70-7.69 (m, 1H), 7.65-7.60 (m, 3H), 7.43-7.31 (m, 8H), 7.08-7.03 (m, 8H), 6.67-6.63 (m, 4H), 6.55-6.48 (m, 5H), 6.17-6.13 (m, 8H)

### Synthesis Example 20

### Synthesis of Compound 39

Compound 39 was synthesized in the same manner as in Synthesis Example 3, except that 9-bromophenathrene was used instead of 9,9-dimethyl-2-iodofluorene in the synthesis of Intermediate 41-5. The obtained compound was confirmed by MS/FAB and ¹H NMR.

C₆₀H₄₁N₃O: calc. 819.32. found 819.36

¹H NMR (CDCl₃, 400 MHz) δ (ppm) 8.66-8.64 (m, 1H), 8.27-8.25 (m, 1H), 8.17-8.15 (m, 1H), 7.76-7.75 (m, 1H), 7.68-7.54 (m, 6H), 7.50-7.43 (m, 2H), 7.41-7.38 (m, 2H), 7.35-7.33 (m, 2H), 7.08-7.04 (m, 8H), 6.66-6.58 (m, 6H), 6.53-6.48 (m, 3H), 6.16-6.13 (m, 8H)

### Synthesis Example 21

### Synthesis of Compound 42

Compound 42 was synthesized in the same manner as in Synthesis Example 3, except that 3-bromo-9-phenyl-9H-carbazole was used instead of 9,9-dimethyl-2-iodofluorene in the synthesis of Intermediate 41-5. The obtained compound was confirmed by MS/FAB and ¹H NMR.

C₆₄H₄₄N₄O: calc. 884.35. found 884.42

¹H NMR (CDCl₃, 400 MHz) δ (ppm) 8.37-8.31 (m, 1H), 7.87-7.86 (m, 1H), 7.72-7.71 (m, 1H), 7.65-7.61 (m, 3H), 7.51-7.48 (m, 4H), 7.41-7.27 (m, 8H), 7.08-7.01 (m, 9H), 6.67-6.63 (m, 4H), 6.57-6.56 (m, 1H), 6.53-6.48 (m, 4H), 6.16-6.12 (m, 81-1)

### Synthesis Example 22

### Synthesis of Compound 44

Compound 44 was synthesized in the same manner as in Synthesis Example 3, except that 3-(3-bromo-5-(pyridin-3-yl)phenyl)pyridine was used instead of 9,9-dimethyl-2-iodofluorene in the synthesis of Intermediate 41-5. The obtained compound was confirmed by MS/FAB and ¹H NMR.

C₆₂H₄₃N₅O: calc. 873.34. found 873.40

¹H NMR (CDCl₃, 400 MHz) δ (ppm) 8.89-8.88 (m, 2H), 8.67-8.66 (m, 2H), 8.10-8.08 (m, 2H), 7.96-7.95 (m, 2H), 7.73 (s, 1H), 7.65-7.62 (m, 4H), 7.53-7.50 (m, 2H), 7.37-7.35 (m, 3H), 7.08-7.04 (m, 8H), 6.66-6.64 (m, 4H), 6.55-6.49 (m, 5H), 6.16-6.12 (m, 8H)

### Synthesis Example 23

### Synthesis of Compound 45

### Synthesis of Intermediate 45-6

Intermediate 45-6 below was synthesized in the same manner as in the synthesis of Intermeidate 41-5, except that iodobenzene was used instead of 9,9-dimethyl-2-iodofluorene in the synthesis of Intermediate 41-5.

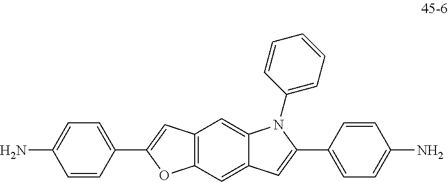 [SMILES: Nc1ccc(-c2cc3cc4c(cc(-c5ccc(N)cc5)n4-c4ccccc4)cc3o2)cc1]

### Synthesis of Intermediate 45-7

Intermediate 45-7 below was synthesized in the same manner as in the synthesis of Intermediate 19-7, except that Intermediate 45-6 was used instead of Intermediate 3-6 and 1-bromonaphthalene was used instead of 4-bromo-biphenyl.

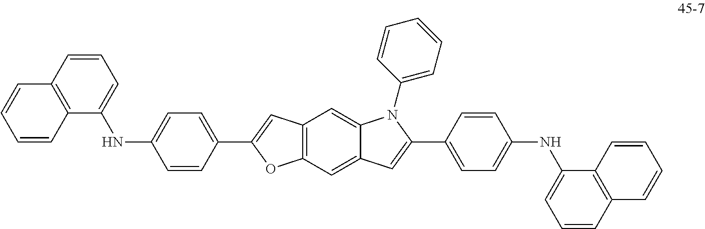 [SMILES: c1ccc(-n2c(-c3ccc(Nc4cccc5ccccc45)cc3)cc3cc4oc(-c5ccc(Nc6cccc7ccccc67)cc5)cc4cc32)cc1]

### Synthesis of Compound 45

Compound 45 was synthesized in the same manner as in the synthesis process of Compound 3, except that Intermediate 45-7 was used instead of Intermediate 3-6. The obtained compound was confirmed by MS/FAB and ¹H NMR.

C₆₀H₄₁N₃O: calc. 819.32. found 819.37

¹H NMR (CDCl₃, 400 MHz) δ (ppm) 8.17-8.15 (m, 2H), 7.87-7.85 (m, 2H), 7.70-7.69 (m, 1H), 7.66-7.60 (m, 3H), 7.49-7.31 (m, 13H), 7.25-7.21 (m, 3H), 7.06-7.01 (m, 4H), 6.74-6.72 (m, 2H), 6.65-6.61 (m, 2H), 6.55-6.54 (m, 1H), 6.40-6.37 (m, 2H), 6.30-6.27 (m, 2H), 6.07-6.04 (m, 4H)

### Synthesis Example 24

### Synthesis of Compound 48

Compound 48 was synthesized in the same manner as in Synthesis Example 3, except that iodobenzene was used instead of 9,9-dimethyl-2-iodofluorene in the synthesis process of Intermediate 41-5 and 4-bromobenzonitrile was used instead of iodobenzene in the synthesis process of Compound 41. The obtained compound was confirmed by MS/FAB and ¹H NMR.

C₅₆H₃₃N₇O: calc. 819.27. found 819.33

¹H NMR (CDCl₃, 400 MHz) δ (ppm) 7.70-7.69 (m, 1H), 7.65-7.60 (m, 3H), 7.43-7.31 (m, 16H), 6.63-6.79 (m, 8H), 6.69-6.62 (m, 4H), 6.55-6.52 (m, 1H)

### Synthesis Example 25

### Synthesis of Compound 51

Compound 51 was synthesized in the same manner as in Synthesis Example 3, except that N-(4-bromophenyl)-N-phenylbenzenamine was used instead of 9,9-dimethyl-2-iodofluorene in the synthesis process of Intermediate 41-5. The obtained compound was confirmed by MS/FAB and ¹H NMR.

C₆₄H₄₆N₄O: calc. 886.36. found 886.40

¹H NMR (CDCl₃, 400 MHz) δ (ppm) 7.70-7.69 (m, 1H), 7.64-7.60 (m, 3H), 7.38-7.34 (m, 3H), 7.08-7.03 (m, 12H), 6.85-6.81 (m, 2H), 6.75-6.71 (m, 2H), 6.67-6.63 (m, 6H), 6.55-6.48 (m, 5H), 6.16-6.13 (m, 12H)

### Synthesis Example 26

### Synthesis of Compound 53

Compound 53 was synthesized according to Reaction Scheme 5 below:

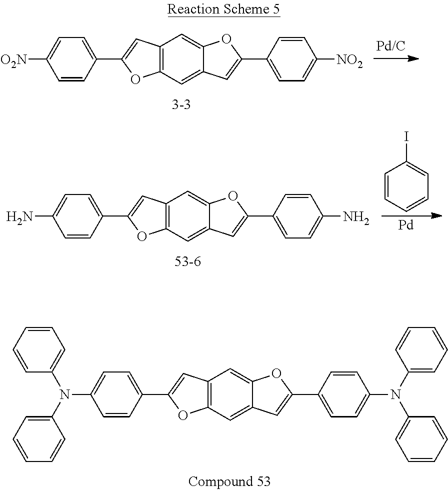

### Synthesis of Intermediate 53-6

Intermediate 53-6 was synthesized in the same manner as in the synthesis process of Intermediate 3-6, except that Intermediate 3-3 was used instead of Intermediate 3-5.

### Synthesis of Compound 53

Compound 53 was synthesized in the same manner as in the synthesis process of Compound 3, except that Intermediate 53-6 was used instead of Intermediate 3-6. The obtained compound was confirmed by MS/FAB and ¹H NMR.

C₄₆H₃₂N₂O₂: calc. 644.24. found 644.29

¹H NMR (CDCl₃, 400 MHz) δ (ppm) 7.70-7.69 (m, 2H), 7.65-7.61 (m, 4H), 7.08-7.03 (m, 10H), 6.66-6.63 (m, 4H), 6.52-6.48 (m, 4H), 6.17-6.13 (m, 8H)

### Synthesis Example 27

### Synthesis of Compound 56

Compound 56 was synthesized in the same manner as in Synthesis Example 26, except that 4-bromodiphenyl was used instead of iodobenzene in the synthesis process of Compound 53. The obtained compound was confirmed by MS/FAB and ¹H NMR.

C₇₀H₄₈N₂O₂: calc. 948.37. found 948.44

¹H NMR (CDCl₃, 400 MHz) δ (ppm) 7.70-7.69 (m, 2H), 7.65-7.61 (m, 12H), 7.53-7.48 (m, 8H), 7.45-7.39 (m, 12H), 7.05-7.04 (m, 2H), 6.86-6.82 (m, 8H), 6.65-6.62 (m, 4H)

### Synthesis Example 28

### Synthesis of Compound 57

### Synthesis of Intermediate 57-7

Intermediate 57-7 below was synthesized in the same manner as in the synthesis process of Compound 53, except that 12.00 mmol of Intermediate 53-6 was used and 3-bromo-9-ethyl-9H-carbazole was used instead of iodobenzene.

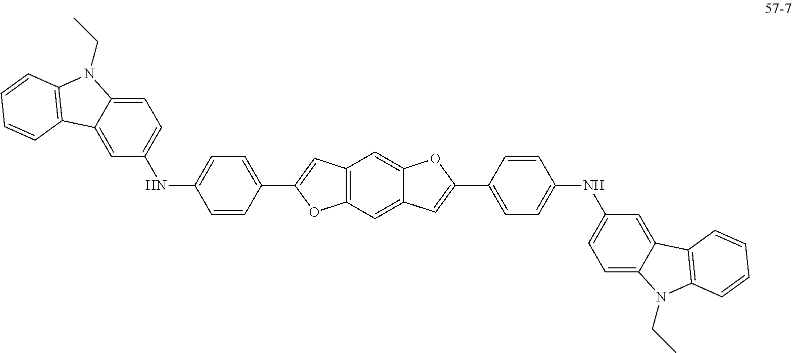 [SMILES: CCn1c2ccccc2c2cc(Nc3ccc(-c4cc5cc6oc(-c7ccc(Nc8ccc9c(c8)c8ccccc8n9CC)cc7)cc6cc5o4)cc3)ccc21]

### Synthesis of Compound 57

Compound 57 was synthesized in the same manner as in the synthesis process of Compound 53, except that Intermediate 57-7 was used instead of Intermediate 53-6. The obtained compound was confirmed by MS/FAB and ¹H NMR.

C₆₂H₄₆N₄O₂: calc. 878.36. found 878.41

¹H NMR (CDCl₃, 400 MHz) δ (ppm) 8.07-8.04 (m, 2H), 7.71-7.70 (m, 2H), 7.65-7.61 (m, 4H), 7.46-7.30 (m, 10H), 7.08-7.04 (m, 6H), 6.71-6.63 (m, 8H), 6.31-6.28 (m, 4H), 4.35-4.31 (m, 4H), 1.43-1.39 (m, 6H)

### Synthesis Example 29

### Synthesis of Compound 58

### Synthesis of Intermediate 58-7

Intermediate 58-7 below was synthesized in the same manner as in the synthesis process of Compound 53, except that 12.00 mmol of Intermediate 53-6 was used and 4-bromodiphenyl was used instead of iodobenzene.

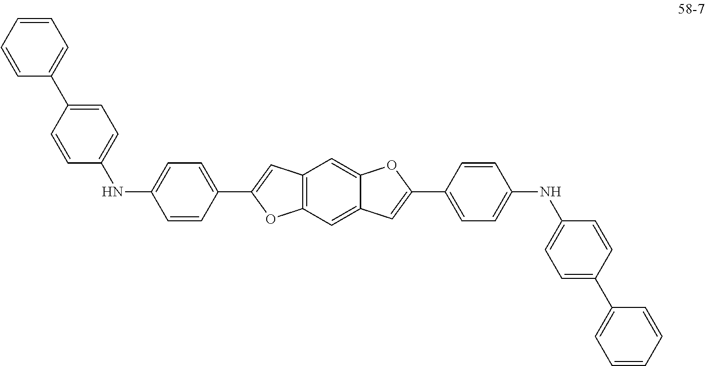 [SMILES: c1ccc(-c2ccc(Nc3ccc(-c4cc5cc6oc(-c7ccc(Nc8ccc(-c9ccccc9)cc8)cc7)cc6cc5o4)cc3)cc2)cc1]

### Synthesis of Compound 58

Compound 58 was synthesized in the same manner as in the synthesis process of Compound 53, except that Intermediate 58-7 was used instead of Intermediate 53-6 and 9,9-dimethyl-2-iodofluorene was used instead of iodobenzene. The obtained compound was confirmed by MS/FAB and ¹H NMR.

C₇₆H₅₆N₂O₂: calc. 1028.43. found 1028.47

¹H NMR (CDCl₃, 400 MHz) δ (ppm) 7.78-7.76 (m, 2H), 7.70-7.69 (m, 2H), 7.65-7.61 (m, 8H), 7.56-7.49 (m, 6H), 7.47-7.30 (m, 8H), 7.12-7.09 (m, 4H), 7.05-7.04 (m, 2H), 6.71-6.69 (m, 2H), 6.58-6.49 (m, 8H), 6.42-6.41 (m, 2H), 1.61 (m, 12H)

### Synthesis Example 30

### Synthesis of Compound 63

### Synthesis of Intermediate 63-7

Intermediate 63-7 below was synthesized in the same manner as in the synthesis process of Compound 61, except that 12.00 mmol of Intermediate 61-6 was used and 4-bromopyridine was used instead of iodobenzene.

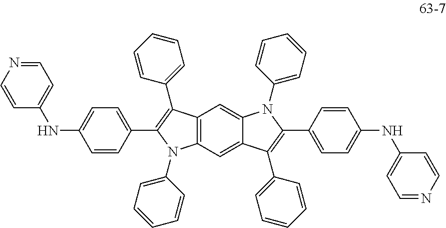 [SMILES: c1ccc(-c2c(-c3ccc(Nc4ccncc4)cc3)n(-c3ccccc3)c3cc4c(-c5ccccc5)c(-c5ccc(Nc6ccncc6)cc5)n(-c5ccccc5)c4cc23)cc1]

### Synthesis of Compound 63

Compound 63 was synthesized in the same manner as in the synthesis process of Compound 61, except that Intermediate 63-7 was used instead of Intermediate 61-6. The obtained compound was confirmed by MS/FAB and ¹H NMR.

C₆₈H₄₈N₆: calc. 948.39. found 948.44

¹H NMR (CDCl₃, 400 MHz) δ (ppm) 8.46-8.45 (m, 4H), 8.16 (s, 2H), 7.48-7.33 (m, 20H), 7.07-6.98 (m, 4H), 6.93-6.91 (m, 4H), 6.66-6.63 (m, 2H), 6.49-6.47 (m, 4H), 6.43-6.40 (m, 4H), 6.33-6.31 (m, 4H)

### Synthesis Example 31

### Synthesis of Compound 68

Compound 68 was synthesized according to Reaction Scheme 6 below:

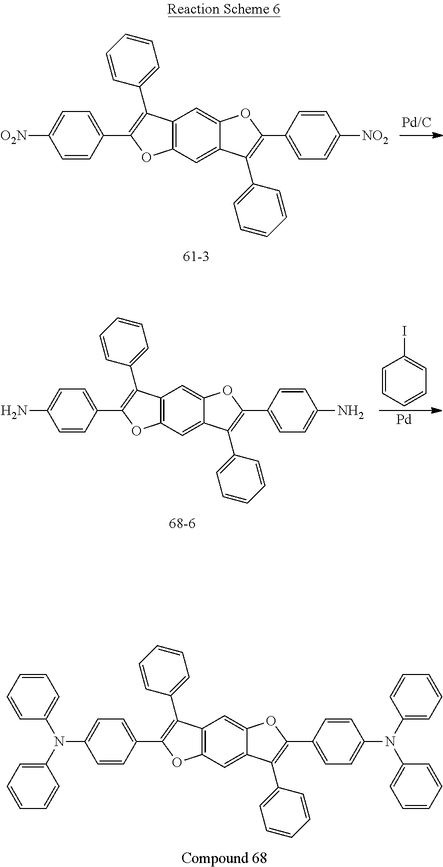

### Synthesis of Intermediate 68-6

Intermediate 68-6 was synthesized in the same manner as in the synthesis process of Intermediate 3-6, except that Intermediate 61-3 was used instead of Intermediate 3-5.

### Synthesis of Compound 68

Compound 68 was synthesized in the same manner as in the synthesis process of Compound 3, excpet that Intermediate 68-6 was used instead of Intermediate 3-6. The obtained compound was confirmed by MS/FAB and ¹H NMR.

C₅₈H₄₀N₂O₂: calc. 796.30. found 796.33

¹H NMR (CDCl₃, 400 MHz) δ (ppm) 8.01 (s, 2H), 7.59-7.55 (m, 4H), 7.53-7.49 (m, 4H), 7.43-7.35 (m, 6H), 7.08-7.01 (m, 8H), 6.66-6.63 (m, 4H), 6.47-6.44 (m, 4H), 6.16-6.13 (m, 8H)

### Comparative Example 1

### Synthesis of Compound A

Compound A was synthesized according to Reaction Scheme A below:

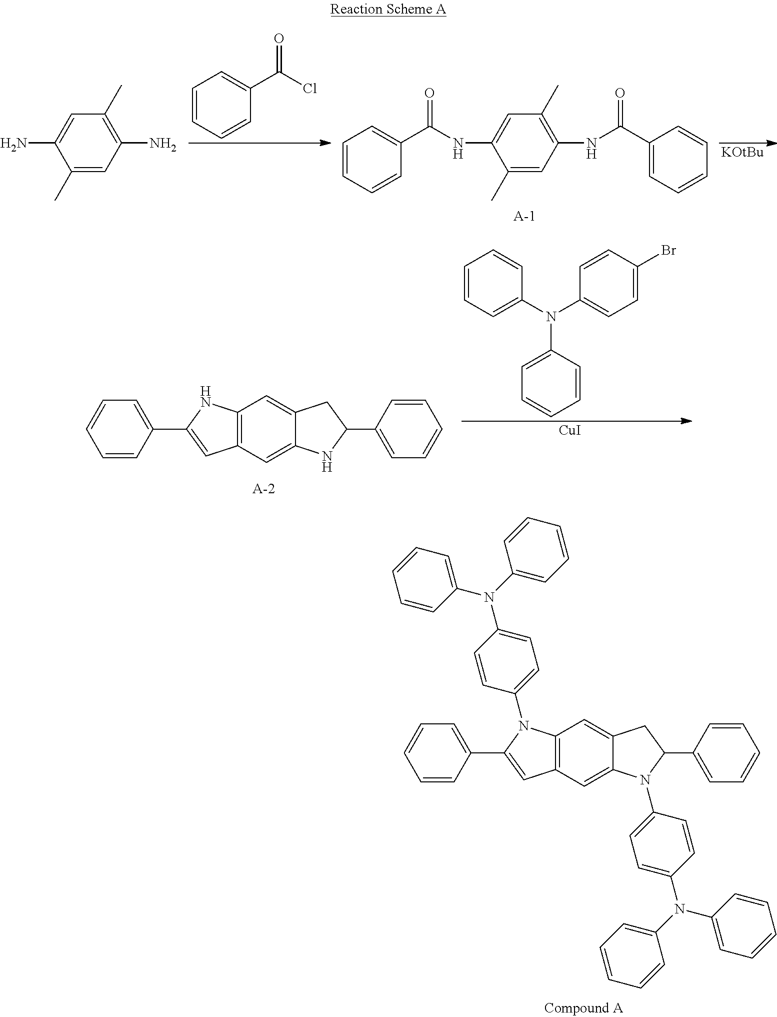

### Synthesis of Intermediate A-1

10 g (73.4 mmol) of 2,5-dimethyl-1,4-phenylenediamine was dissolved in 50 ml of methylenechloride, and 25 ml of triethylamine was then added to the mixed solution. The temperature of the reaction solution was raised to 80° C. and 17 ml (0.147 mmol) of benzoyl chloride was slowly added thereto and the resultant solution was stirred at 30° C. for 1 hour. The reaction solution was evaporated and recrystallized with DMF. The obtained solid was washed with acetone to obtain 23.4 g of Intermediate A-1 (yield 93%). The obtained compound was confirmed by MS/FAB.

C₂₂H₂₀N₂O₂: calc. 344.15. found 344.20

### Synthesis of Intermediate A-2

4.5 g (12.0 mmol) of Intermediate A-1 and 13.5 g (120 mmol) of KotBu were put in an autoclave at 340° C. and 5 Mpa for 2 hours. The reaction product was cooled down to room temperature and then neutralized with distilled water and filtered. The obtained reaction product was purified with acetone for 2 days by using a Soxhlet extracter to obtain 0.94 g of Intermediate A-2 (yield 21%). The obtained compound was confirmed by MS/FAB.

C₂₂H₁₆N₂: calc. 308.13. found 308.17

### Synthesis of Compound A

3.08 g (10.0 mmol) of Intermeidate A-2, 9.73 g (30.0 mmol) of 4-bromotriphenylamine, 0.76 g (4.0 mmol) of CuI, 0.10 g (4.0 mmol) of 18-crown-6, and 11.1 g (80.0 mmol) of K₂CO₃ were dissolved in 50 ml of 1,3-dimethyl-3,4,5,6-tetrahydro-2(1H)-pyrimidinone (DMPU) and the mixed solution was then stirred at 170° C. for 20 hours. The reaction solution was cooled down to room temperature and then extracted three times with 100 ml of water and 100 ml of dichloromethane. The obtained organic layer was dried with magnesium sulfate and a solvent was evaporated therefrom to obtain a crude product. The crude product was purified with silicagel column chromatography to obtain 3.74 g of Compound A (yield 47%). The obtained compound was confirmed by MS/FAB and ¹H NMR.

C₅₈H₄₂N₄: calc. 794.34. found 795.35

¹H NMR (CD₂Cl₂, 400 MHz) δ (ppm) 7.91 (d, 4H), 7.45 (s, 2H), 7.38-7.08 (m, 38H); ¹³C NMR (CD₂Cl₂, 100 MHz) δ (ppm) 129.7, 129.3, 129.0, 128.6, 127.2, 126.4, 125.2, 124.5, 124.1, 123.5, 123.1, 122.2, 103.2, 100.4, 99.3, 98.3

### Example 1

As an anode, a 15 Ω/cm² (1200 Å) Coming ITO glass substrate was cut to a size of 50 mm×50 mm×0.7 mm, washed with ultrasonic waves in isopropyl alcohol and pure water for 5 minutes each, and then cleaned with UV and ozone for 30 minutes. The ITO glass substrate was mounted on a vacuum depositor.

2-TNATA was deposited on the ITO glass substrate to form a HIL having a thickness of 600 Å and Compound 3 was then deposited on the HIL to form a HTL having a thickness of 300 Å.

Next, 9,10-di(naphthalene-2-yl)anthracene (ADN) and 4,4′-bis(2-(4-(N,N-diphenylamino)phenyl)vinyl)biphenyl (DPAVBi) were co-deposited on the HTL at a weight ratio of 98:2 to form an EML having a thickness of 300 Å.

Thereafter, Alq₃ was deposited on the EML to form an ETL having a thickness of 300 Å, LiF was deposited on the ETL to form an EIL having a thickness of 10 Å, and Al was deposited on the EIL to form a second electrode (cathode) having a thickness of 3,000 Å, thereby completing the manufacture of an OLED.

### Example 2

An OLED was manufactured in the same manner as in Example 1, except that Compound 19 was used instead of Compound 3 in the formation of the HTL.

### Example 3

An OLED was manufactured in the same manner as in Example 1, except that Compound 61 was used instead of Compound 3 in the formation of the HTL.

### Example 4

An OLED was manufactured in the same manner as in Example 1, except that Compound 64 was used instead of Compound 3 in the formation of the HTL.

### Example 5

An OLED was manufactured in the same manner as in Example 1, except that 4,4′-bis[N-(1-naphthyl)-N-phenylamino]biphenyl (NPB) was used instead of Compound 3 in the formation of the HTL and Compound 41 was used instead of DPAVBi in the formation of the EML.

### Example 6

An OLED was manufactured in the same manner as in Example 1, except that NPB was used instead of Compound 3 in the formation of the HTL and Compound 68 was used instead of DPAVBi in the formation of the EML.

### Example 7

An OLED was manufactured in the same manner as in Example 1, except that Compound 19 was used instead of Compound 3 in the formation of the HTL and Compound 41 was used instead of DPAVBi in the formation of the EML.

### Comparative Example 1

An OLED was manufactured in the same manner as in Example 1, except that NPB was used instead of Compound 3 in the formation of the HTL.

### Comparative Example 2

An OLED was manufactured in the same manner as in Example 1, except that Compound A was used instead of Compound 3 in the formation of the HTL.

### Comparative Example 3

An OLED was manufactured in the same manner as in Comparative Example 1, except that Compound A was used instead of DPAVBi in the formation of the EML.

### Evaluation Example 1

Driving voltage, current density, brightness, emission color, efficiency, and half lifetime (@100 mA/cm²) of each of the OLEDs of Examples 1 through 7 and Comparative Examples 1 through 3 were evaluated using PR650 Spectroscan Source Measurement Unit (available from PhotoResearch), and the results are shown in Table 1 below.

TABLE 1

| | Hole | Emission | Driving | Cureent | | | | Half |
|---|---|---|---|---|---|---|---|---|
| | transport | layer | voltage | density | Brightness | efficiency | Emission | lifetime |
| | layer | (dopant) | (V) | (mA/cm²) | (cd/m²) | (cd/A) | color | (hr) |
| Example 1 | Compound 3 | DPAVBi | 6.13 | 50 | 2,890 | 5.78 | blue | 295 |
| Example 2 | Compound | DPAVBi | 6.15 | 50 | 2,835 | 5.67 | blue | 289 |
| | 19 | | | | | | | |
| Example 3 | Compound | DPAVBi | 6.21 | 50 | 2,880 | 5.76 | blue | 297 |
| | 61 | | | | | | | |
| Example 4 | Compound | DPAVBi | 6.25 | 50 | 2,825 | 5.65 | blue | 306 |
| | 64 | | | | | | | |
| Example 5 | NPB | Compound | 6.67 | 50 | 3,185 | 6.37 | blue | 246 |
| | | 41 | | | | | | |
| Example 6 | NPB | Compound | 6.76 | 50 | 3,115 | 6.23 | blue | 235 |
| | | 68 | | | | | | |
| Example 7 | Compound | Compound | 6.07 | 50 | 3,280 | 6.56 | blue | 352 |
| | 19 | 41 | | | | | | |
| Comparative | NPB | DPAVBi | 7.35 | 50 | 2,065 | 4.13 | blue | 145 |
| Example 1 | | | | | | | | |
| Comparative | Compound A | DPAVBi | 5.87 | 50 | 2,630 | 5.26 | blue | 208 |
| Example 2 | | | | | | | | |
| Comparative | NPB | Compound A | 6.57 | 50 | 1,315 | 2.63 | blue | 167 |
| Example 3 | | | | | | | | |

From the results shown in Table 1, it is confirmed that the OLEDs of Examples 1 through 7 have excellent driving voltage, higher luminance, higher efficiency, higher color purity, and longer lifetime, as compared to the OLEDs of Comparative Examples 1 through 3.

Compound A may have, but is not limited to, a structure in which a diarylamino group is linked to N of the core via a phenyl group. In this regard, an absolute value of a HOMO energy level is decreased by an electron donating effect by conjugation of the diarylamino group and thus a smooth Dexter energy transfer from a host of the EML may not occur. Therefore, an OLED that includes an EML including Compound A may have reduced luminous efficiency.

As described above, according to the one or more embodiments of the present invention, a condensed-cyclic compound represented by Formula 1 may exhibit high heat resistance, high electrical stability, and a high color purity of blue emission and thus an OLED including the condensed-cyclic compound represented by Formula 1 may have low driving voltage, high brightness, high efficiency, and long lifetime.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A condensed-cyclic compound represented by Formula 1 below: 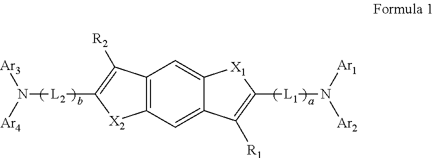
wherein X₁ is N(R₁₁) or O; X₂ is N(R₁₂) or O;
R₁, R₂, R₁₁, and R₁₂ are each independently hydrogen, deuterium, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ aryl group, a substituted or unsubstituted C₅-C₆₀ aryloxy group, a substituted or unsubstituted C₅-C₆₀ arylthio group, a substituted or unsubstituted C₂-C₆₀ heteroaryl group, —N(R₃₁)(R₃₂), or —Si(R₃₃)(R₃₄)(R₃₅);
L₁ and L₂ are each independently a substituted or unsubstituted C₅-C₆₀ arylene group or a substituted or unsubstituted C₂-C₆₀ heteroarylene group;
Ar₁ through Ar₄ are each independently a substituted or unsubstituted C₅-C₆₀ aryl group or a substituted or unsubstituted C₂-C₆₀ heteroaryl group;
a and b are each independently an integer of 1 to 5; and
R₃₁ through R₃₅ are each independently hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ aryl group, a substituted or unsubstituted C₅-C₆₀ aryloxy group, a substituted or unsubstituted C₅-C₆₀ arylthio group, or a substituted or unsubstituted C₂-C₆₀ heteroaryl group.

2. The condensed-cyclic compound of claim 1, wherein at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₆₀ cycloalkyl group, the substituted C₅-C₆₀ aryl group, the substituted C₅-C₆₀ aryloxy group, the substituted C₅-C₆₀ arylthio group, the substituted C₂-C₆₀ heteroaryl group, the substituted C₅-C₆₀ arylene group, and the substituted C₂-C₆₀ heteroarylene group is selected from deuterium; a halogen atom; a hydroxyl group; a nitro group; a cyano group; an amino group; an amidino group; hydrazine; hydrazone; a carboxyl group or a salt thereof; a sulfonic acid group or a salt thereof; a phosphoric acid or a salt thereof; a C₁-C₆₀ alkyl group; a C₂-C₆₀ alkenyl group; a C₂-C₆₀ alkynyl group; a C₆-C₆₀ aryl group; a C₂-C₆₀ heteroaryl group; a C₁-C₆₀ alkyl group, a C₂-C₆₀ alkenyl group, a C₂-C₆₀ alkynyl group, a C₅-C₆₀ aryl group, a C₂-C₆₀ heteroaryl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a phenyl group, and a naphthyl group; —N(Q₁₁)(Q₁₂); and —Si(Q₁₃)(Q₁₄)(Q₁₅); and
wherein Q₁₁ through Q₁₅ are each independently hydrogen; a C₁-C₆₀ alkyl group; a C₅-C₆₀ aryl group; a C₅-C₆₀ aryl group that is substituted with deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₆₀ alkyl group, or a C₁-C₆₀ alkoxy group; a C₂-C₆₀ heteroaryl group; or a C₂-C₆₀ heteroaryl group that is substituted with deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₆₀ alkyl group, or a C₁-C₆₀ alkoxy group.

3. The condensed-cyclic compound of claim 1, wherein R₁, R₂, R₁₁, and R₁₂ are each independently hydrogen, deuterium, a substituted or unsubstituted C₁-C₂₀ alkyl group, a substituted or unsubstituted C₁-C₂₀ alkoxy group, a substituted or unsubstituted phenyl group, a substituted or unsubstituted pentalenyl group, a substituted or unsubstituted indenyl group, a substituted or unsubstituted naphtyl group, a substituted or unsubstituted azulenyl group, a substituted or unsubstituted heptalenyl group, a substituted or unsubstituted indacenyl group, a substituted or unsubstituted acenaphtyl group, a substituted or unsubstituted fluorenyl group, a substituted or unsubstituted spiro-fluorenyl group, a substituted or unsubstituted phenalenyl group, a substituted or unsubstituted phenanthrenyl group, a substituted or unsubstituted anthryl group, a substituted or unsubstituted fluoranthenyl group, a substituted or unsubstituted triphenylenyl group, a substituted or unsubstituted pyrenyl group, a substituted or unsubstituted chrysenyl group, a substituted or unsubstituted naphthacenyl group a substituted or unsubstituted picenyl group, a substituted or unsubstituted perylenyl group, a substituted or unsubstituted pentaphenyl group, a substituted or unsubstituted hexacenyl group, a substituted or unsubstituted pyrrolyl group, a substituted or unsubstituted imidazolyl group, a substituted or unsubstituted pyrazolyl group, a substituted or unsubstituted pyridinyl group, a substituted or unsubstituted pyrazinyl group, a substituted or unsubstituted pyrimidinyl group, a substituted or unsubstituted pyridazinyl group, a substituted or unsubstituted isoindolyl group, a substituted or unsubstituted indolyl group, a substituted or unsubstituted indazolyl group, a substituted or unsubstituted purinyl group, a substituted or unsubstituted quinolinyl group, a substituted or unsubstituted benzoquinolinyl group, a substituted or unsubstituted phthalazinyl group, a substituted or unsubstituted naphthyridinyl group, a substituted or unsubstituted quinoxalinyl group, a substituted or unsubstituted quinazolinyl group, a substituted or unsubstituted cinnolinyl group, a substituted or unsubstituted carbazolyl group, a substituted or unsubstituted phenanthridinyl group, a substituted or unsubstituted acridinyl group, a substituted or unsubstituted phenanthrolinyl group, a substituted or unsubstituted phenazinyl group, a substituted or unsubstituted benzooxazolyl, a substituted or unsubstituted benzoimidazolyl group, a substituted or unsubstituted furanyl group, a substituted or unsubstituted benzofuranyl group, a substituted or unsubstituted thiophenyl group, a substituted or unsubstituted benzothiophenyl group, a substituted or unsubstituted thiazolyl group, a substituted or unsubstituted isothiazolyl group, a substituted or unsubstituted benzothiazolyl group, a substituted or unsubstituted isoxazolyl group, a substituted or unsubstituted oxazolyl group, a substituted or unsubstituted triazolyl group, a substituted or unsubstituted tetrazolyl group, a substituted or unsubstituted oxadiazolyl group, a substituted or unsubstituted triazinyl group, a substituted or unsubstituted benzooxazolyl group, a substituted or unsubstituted dibenzopuranyl group, a substituted or unsubstituted dibenzothiophenyl group, or a substituted or unsubstituted benzocarbazolyl group.

4. The condensed-cyclic compound of claim 1, wherein the condensed-cyclic compound is represented by one of Formulae 1A through 1C below: 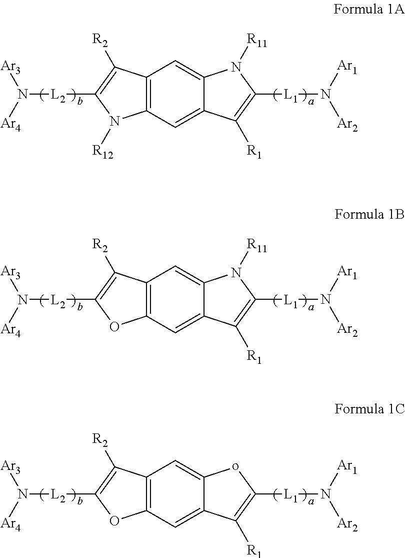
wherein R₁, R₂, R₁₁, and R₁₂, Ar₁ through Ar₄, L₁ and L₂, and a and b are the same as defined in claim 1.

5. The condensed-cyclic compound of claim 1, wherein R₁₁ and R₁₂ are each independently a substituted or unsubstituted C₁-C₂₀ alkyl group or one of Formulae 3-1 through 3-27 below: 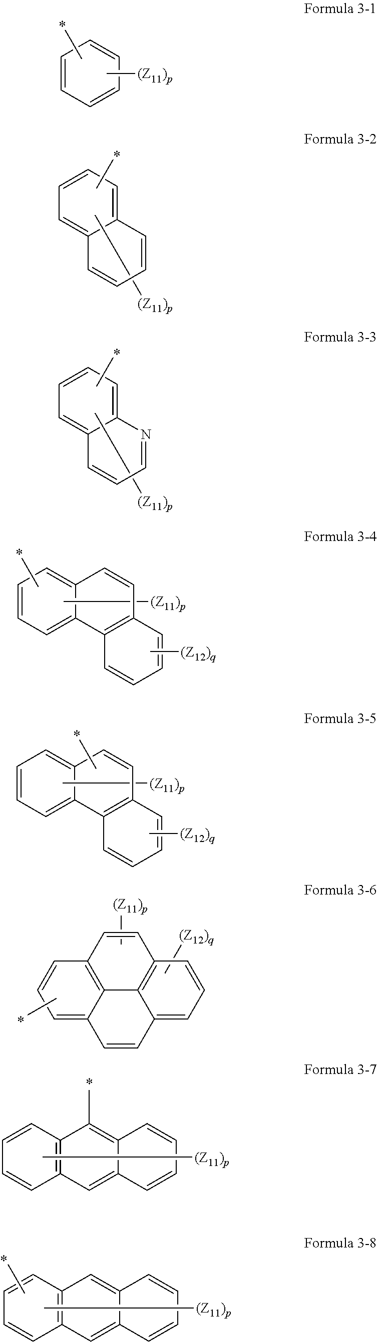 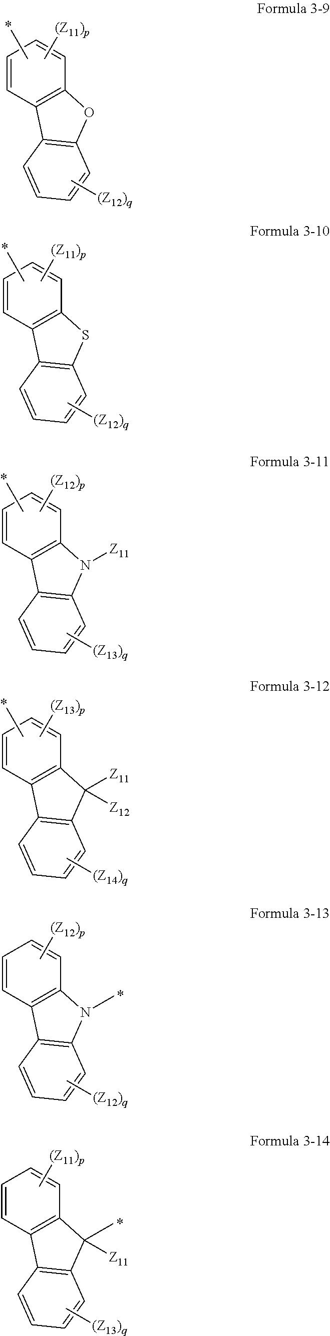 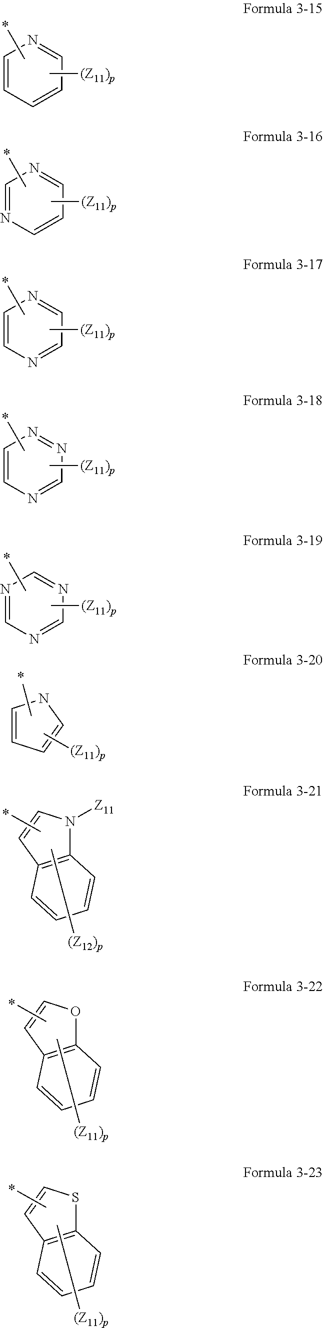 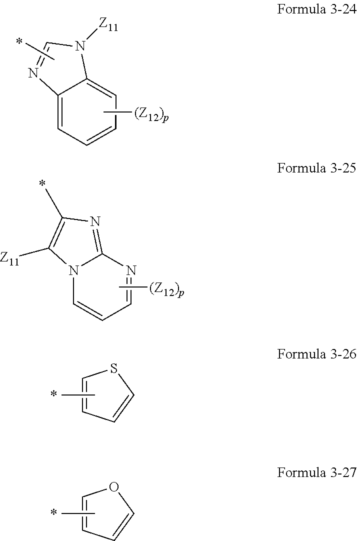
wherein Z₁₁ through Z₁₄ are each independently one of hydrogen; deuterium; a halogen atom; a hydroxyl group; a cyano group; a nitro group; an amino group; an amidino group; hydrazine; hydrazone; a carboxyl group or a salt thereof; a sulfonic acid group or a salt thereof; a phosphoric acid or a salt thereof; a C₁-C₁₀ alkyl group; a C₁-C₁₀ alkoxy group; a C₁-C₁₀ alkyl group and a C₁-C₁₀ alkoxy group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof; a phenyl group; a naphthyl group; a fluorenyl group; a phenanthrenyl group; an anthryl group; a pyrenyl group; a chrysenyl group; a phenyl group, a naphthyl group, a fluorenyl group; a phenanthrenyl group, an anthryl group, a pyrenyl group, and a chrysenyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, and a C₁-C₁₀ alkoxy group; an indolyl group; a benzoimidazolyl group; a carbazolyl group; an imidazolyl group; an imidazolinyl group; an imidazopyridinyl group; an imidazopyrimidinyl group; a pyridinyl group; a pyrimidinyl group; a triazinyl group; a quinolinyl group; an indolyl group, a benzoimidazolyl group, a carbazolyl group, an imidazolyl group, an imidazolinyl group, an imidazopyridinyl group, an imidazopyrimidinyl group, a pyridinyl group, a pyrimidinyl group, a triazinyl group, and a quinolinyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a phenyl group, and a naphthyl group; and —N(Q₁₁)(Q₁₂);
p is an integer of 1 to 9;
q is an integer of 1 to 4; and
Q₁₁ and Q₁₂ are each independently one of a phenyl group; a naphthyl group; a fluorenyl group; a phenanthrenyl group; an anthryl group; a pyrenyl group; a chrysenyl group; and a phenyl group, a naphthyl group, a fluorenyl group, a phenanthrenyl group, an anthryl group, a pyrenyl group, and a chrysenyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, and a C₁-C₁₀ alkoxy group.

6. The condensed-cyclic compound of claim 1, wherein R₁₁ and R₁₂ are each independently one of a methyl group; an ethyl group; a propyl group; a butyl group; a pentyl group; a hexyl group; a heptyl group; a methyl group, an ethyl group, a propyl group, a butyl group, a pentyl group, a hexyl group, and a heptyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof; and Formulae 4-1 through 4-41 below: 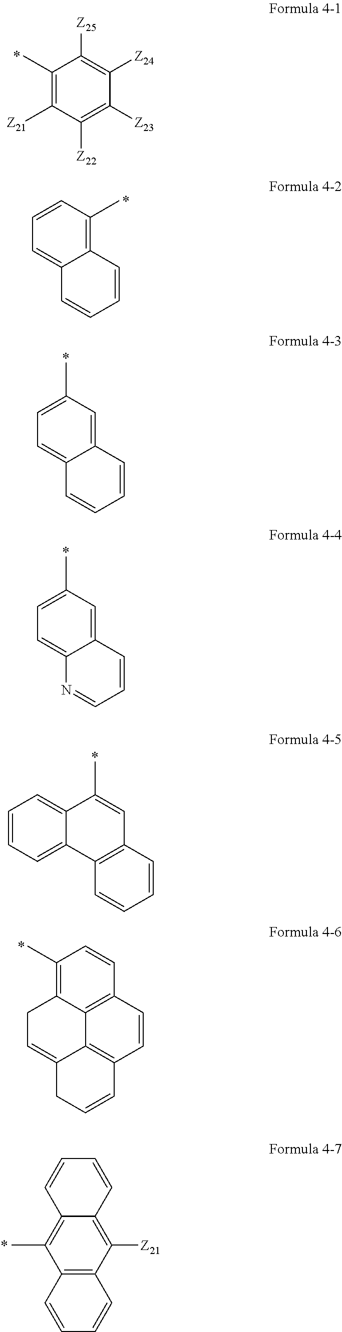 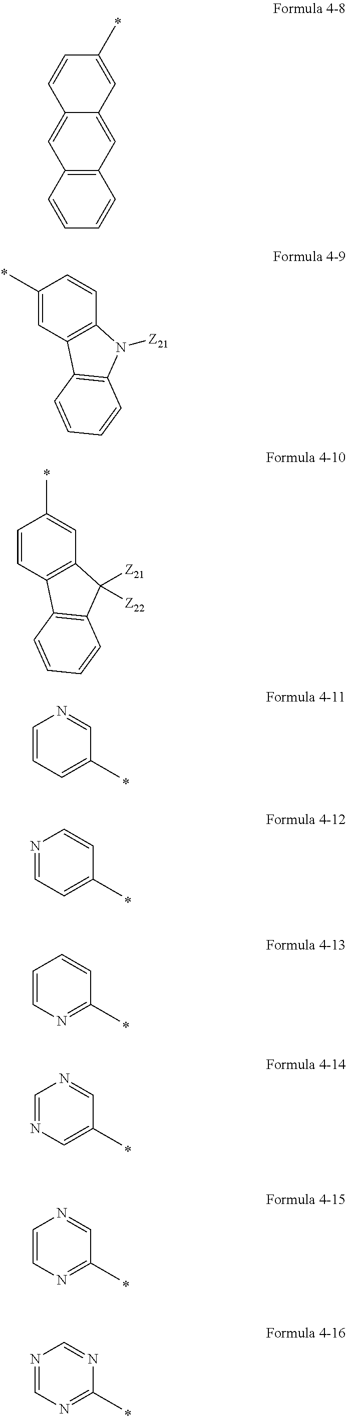 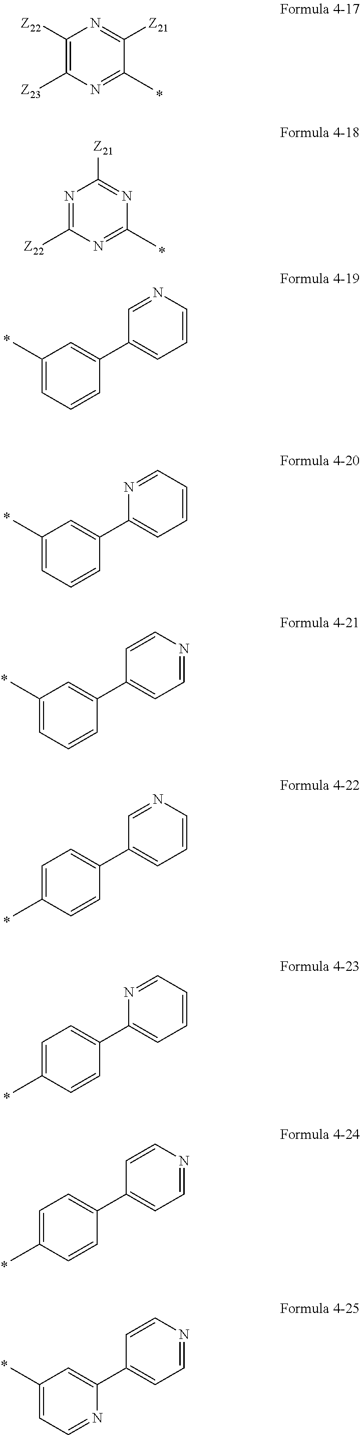 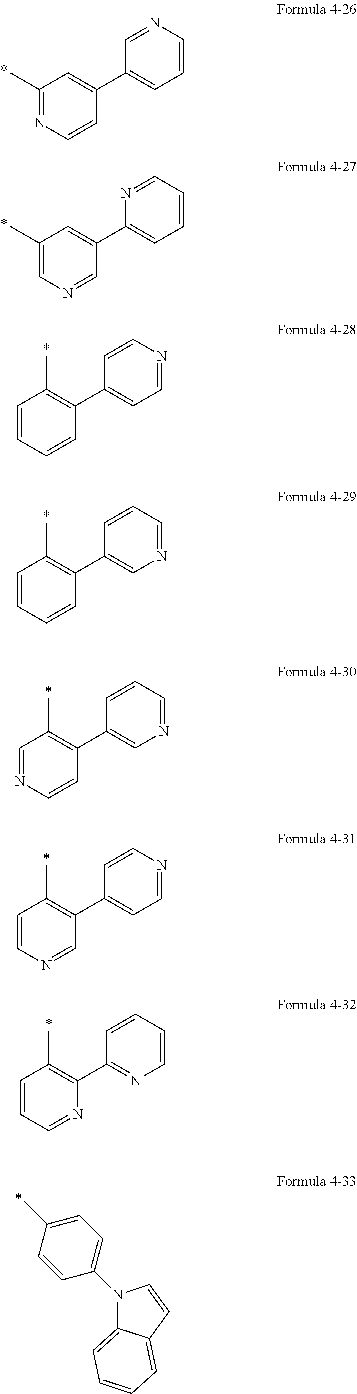 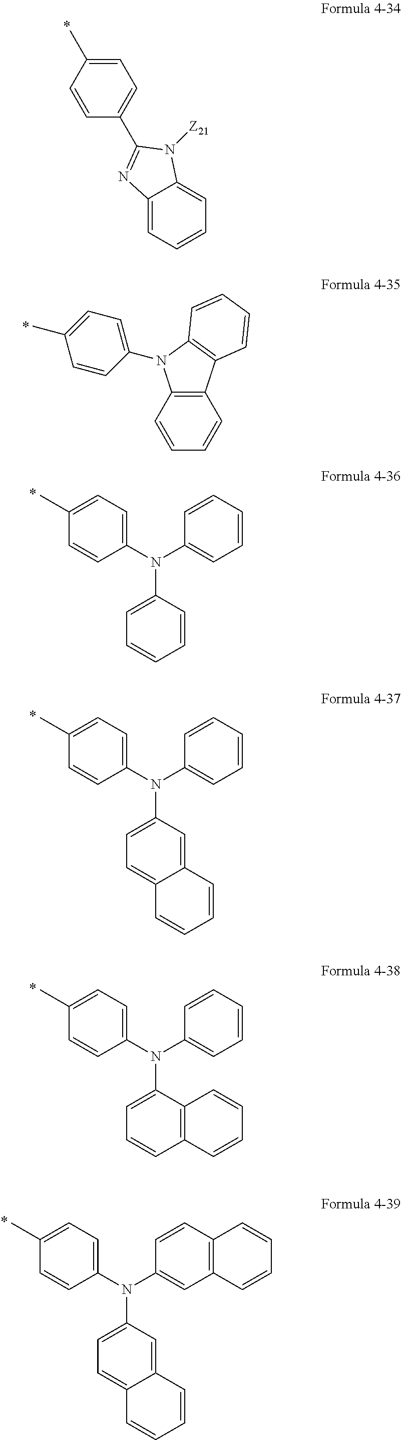 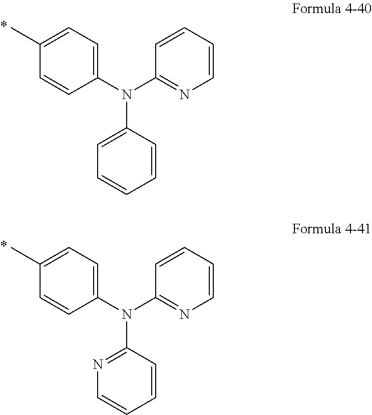 [SMILES: Cc1ccc(N(c2ccccc2)c2ccccn2)cc1 ; Cc1ccc(N(c2ccccn2)c2ccccn2)cc1]
wherein Z₂₁ through Z₂₅ are each independently hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a phenyl group, a naphthyl group, a fluorenyl group, a phenanthrenyl group, an anthryl group, a pyrenyl group, a chrysenyl group, an indolyl group, a benzoimidazolyl group, a carbazolyl group, an imidazolyl group, an imidazolinyl group, an imidazopyridinyl group, an imidazopyrimidinyl group, a pyridinyl group, a pyrimidinyl group, a triazinyl group, or a quinolinyl group.

7. The condensed-cyclic compound of claim 1, wherein R₁ and R₂ are each independently hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a phenyl group, a naphthyl group, a fluorenyl group, a phenanthrenyl group, an anthryl group, or a pyrenyl group.

8. The condensed-cyclic compound of claim 1, wherein L₁ and L₂ are each independently a substituted or unsubstituted phenylene group, a substituted or unsubstituted pentalenylene group, a substituted or unsubstituted indenylene group, a substituted or unsubstituted naphthylene group, a substituted or unsubstituted azulenylene group, a substituted or unsubstituted heptalenylene group, a substituted or unsubstituted indacenylene group, a substituted or unsubstituted acenaphthylene group, a substituted or unsubstituted fluorenylene group, a substituted or unsubstituted phenalenylene group, a substituted or unsubstituted phenanthrenylene group, a substituted or unsubstituted anthrylene group, a substituted or unsubstituted fluoranthenylene group, a substituted or unsubstituted triphenylenylene group, a substituted or unsubstituted pyrenylene group, a substituted or unsubstituted chrysenylene group, a substituted or unsubstituted naphthacenylene group, a substituted or unsubstituted picenylene group, a substituted or unsubstituted perylenylene group, a substituted or unsubstituted pentaphenylene group, a substituted or unsubstituted hexacenylene group, a substituted or unsubstituted pyrrolylene group, a substituted or unsubstituted pyrazolylene group, a substituted or unsubstituted imidazolylene group, a substituted or unsubstituted imidazolinylene group, a substituted or unsubstituted imidazopyridinylene group, a substituted or unsubstituted imidazopyrimidinylene group, a substituted or unsubstituted pyridinylene group, a substituted or unsubstituted pyrazinylene group, a substituted or unsubstituted pyrimidinylene group, a substituted or unsubstituted indolylene group, a substituted or unsubstituted purinylene group, a substituted or unsubstituted quinolinylene group, a substituted or unsubstituted phthalazinylene group, a substituted or unsubstituted indolizinylene group, a substituted or unsubstituted naphthyridinylene group, a substituted or unsubstituted quinazolinylene group, a substituted or unsubstituted cinnolinylene group, a substituted or unsubstituted indazolylene group, a substituted or unsubstituted carbazolylene group, a substituted or unsubstituted phenazinylene group, a substituted or unsubstituted phenanthridinylene group, a substituted or unsubstituted pyranylene group, a substituted or unsubstituted chromenylene group, a substituted or unsubstituted furanylene group, a substituted or unsubstituted benzofuranylene group, a substituted or unsubstituted thiophenylene group, a substituted or unsubstituted henzothiophenylene group, a substituted or unsubstituted isothiazolylene group, a substituted or unsubstituted benzoimidazolylene group, a substituted or unsubstituted isoxazolylene group, a substituted or unsubstituted dibenzothiophenylene group, a substituted or unsubstituted dibenzofuranylene group, a substituted or unsubstituted triazinylene group, or a substituted or unsubstituted oxadiazolylene group.

9. The condensed-cyclic compound of claim 1, wherein L₁ and L₂ are each independently selected from Formulae 2-1 through 2-8 below: 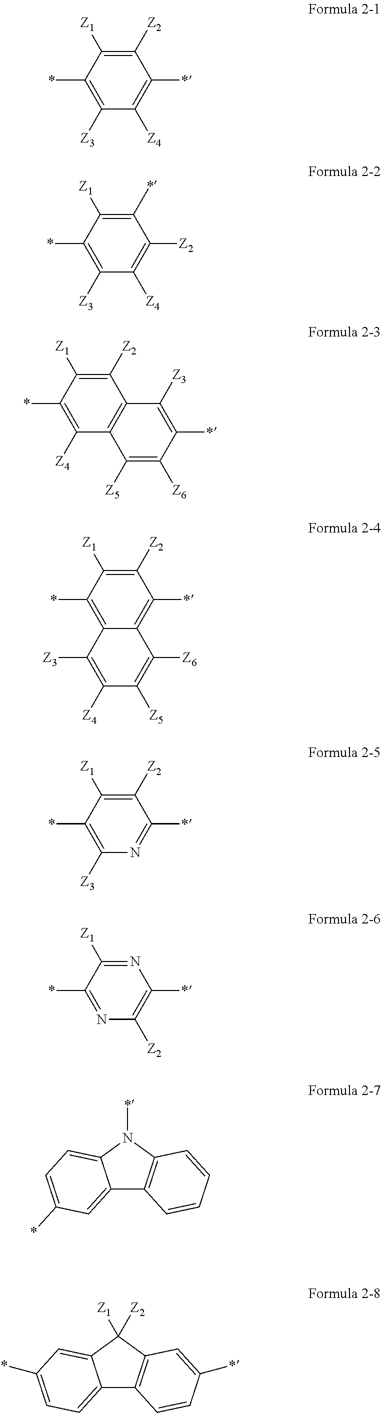
wherein Z₁ through Z₆ are each independently one of hydrogen; deuterium; a halogen atom; a hydroxyl group; a cyano group; a nitro group; an amino group; an amidino group; hydrazine; hydrazone; a carboxyl group or a salt thereof; a sulfonic acid group or a salt thereof; a phosphoric acid or a salt thereof; a C₁-C₁₀ alkyl group; a C₁-C₁₀ alkoxy group; a C₁-C₁₀ alkyl group and a C₁-C₁₀ alkoxy group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof; a phenyl group; a naphthyl group; a fluorenyl group; a phenanthrenyl group; an anthryl group; a pyrenyl group; a chrysenyl group; a phenyl group, a naphthyl group, a fluorenyl group, a phenanthrenyl group, an anthryl group, a pyrenyl group, and a chrysenyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, and a C₁-C₁₀ alkoxy group; an indolyl group; a benzoimidazolyl group; a carbazolyl group; an imidazolyl group; an imidazolinyl group; an imidazopyridinyl group; an imidazopyrimidinyl group; a pyridinyl group; a pyrimidinyl group; a triazinyl group; a quinolinyl group; and an indolyl group, a benzoimidazolyl group, a carbazolyl group, an imidazolyl group, an imidazolinyl group, an imidazopyridinyl group, an imidazopyrimidinyl group, a pyridinyl group, a pyrimidinyl group, a triazinyl group, and a quinolinyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a phenyl group, and a naphthyl group.

10. The condensed-cyclic compound of claim 1, wherein Ar₁ through Ar₄ are each independently a substituted or unsubstituted phenyl group, a substituted or unsubstituted pentalenyl group, a substituted or unsubstituted indenyl group, a substituted or unsubstituted naphtyl group, a substituted or unsubstituted azulenyl group, a substituted or unsubstituted heptalenyl group, a substituted or unsubstituted indacenyl group, a substituted or unsubstituted acenaphtyl group, a substituted or unsubstituted fluorenyl group, a substituted or unsubstituted spiro-fluorenyl group, a substituted or unsubstituted phenalenyl group, a substituted or unsubstituted phenanthrenyl group, a substituted or unsubstituted anthryl group, a substituted or unsubstituted fluoranthenyl group, a substituted or unsubstituted triphenylenyl group, a substituted or unsubstituted pyrenyl group, a substituted or unsubstituted chrysenyl group, a substituted or unsubstituted naphthacenyl group, a substituted or unsubstituted picenyl group, a substituted or unsubstituted perylenyl group, a substituted or unsubstituted pentaphenyl group, a substituted or unsubstituted hexacenyl group, a substituted or unsubstituted pyrrolyl group, a substituted or unsubstituted imidazolyl group, a substituted or unsubstituted pyrazolyl group, a substituted or unsubstituted pyridinyl group, a substituted or unsubstituted pyrazinyl group, a substituted or unsubstituted pyrimidinyl group, a substituted or unsubstituted pyridazinyl group, a substituted or unsubstituted isoindolyl group, a substituted or unsubstituted indolyl group, a substituted or unsubstituted indazolyl group, a substituted or unsubstituted purinyl group, a substituted or unsubstituted quinolinyl group, a substituted or unsubstituted benzoquinolinyl group, a substituted or unsubstituted phthalazinyl group, a substituted or unsubstituted naphthyridinyl group, a substituted or unsubstituted quinoxalinyl group, a substituted or unsubstituted quinazolinyl group, a substituted or unsubstituted cinnolinyl group, a substituted or unsubstituted carbazolyl group, a substituted or unsubstituted phenanthridinyl group, a substituted or unsubstituted acridity group, a substituted or unsubstituted phenanthrolinyl group, a substituted or unsubstituted phenazinyl group, a substituted or unsubstituted benzooxazolyl group, a substituted or unsubstituted benzoimidazolyl group, a substituted or unsubstituted furanyl group, a substituted or unsubstituted benzofuranyl group, a substituted or unsubstituted thiophenyl group, a substituted or unsubstituted benzothiophenyl group, a substituted or unsubstituted thiazolyl, a substituted or unsubstituted isothiazolyl group, a substituted or unsubstituted benzothiazolyl group, a substituted or unsubstituted isoxazolyl group, a substituted or unsubstituted oxazolyl group, a substituted or unsubstituted triazolyl group, a substituted or unsubstituted tetrazolyl group, a substituted or unsubstituted oxadiazolyl group, a substituted or unsubstituted triazinyl group, a substituted or unsubstituted benzooxazolyl group, a substituted or unsubstituted dibenzopuranyl group, a substituted or unsubstituted dibenzothiophenyl group, or a substituted or unsubstituted benzocarbazolyl group.

11. The condensed-cyclic compound of claim 1, wherein Ar₁ through Ar₄ are each independently one of Formulae 3-1 through 3-27 below: 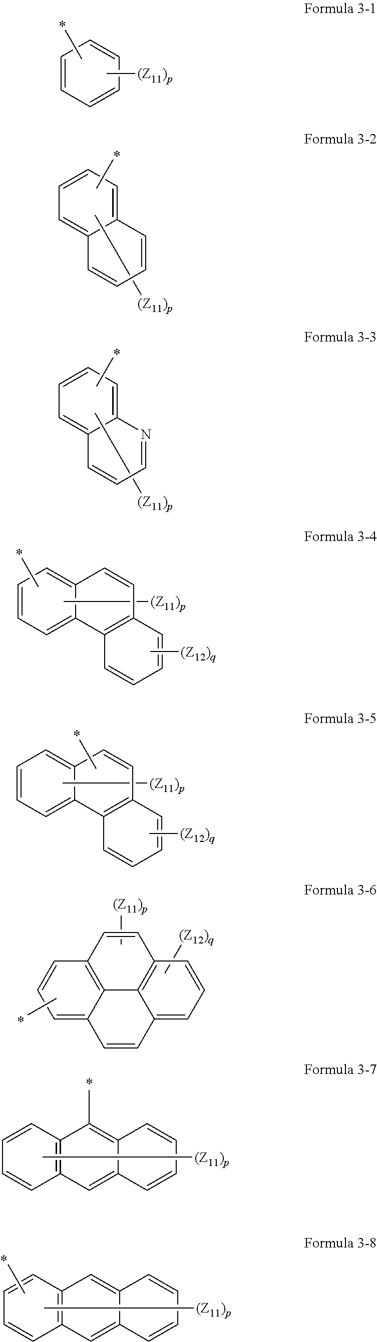 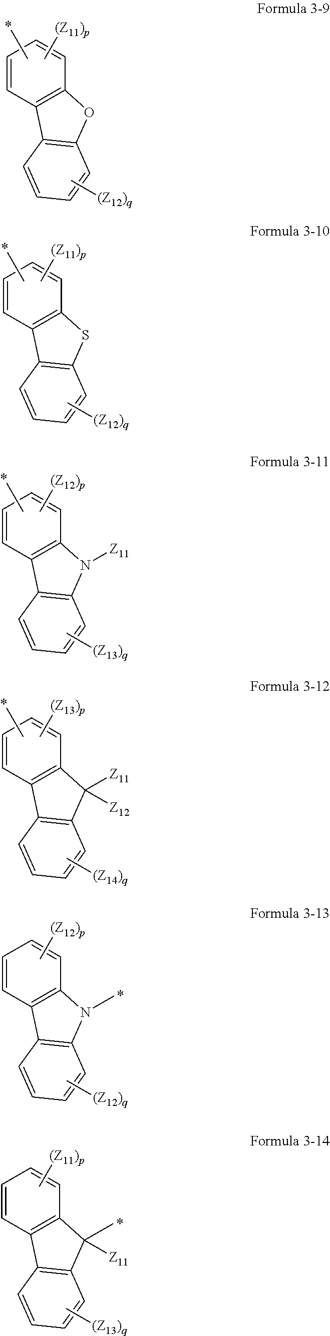 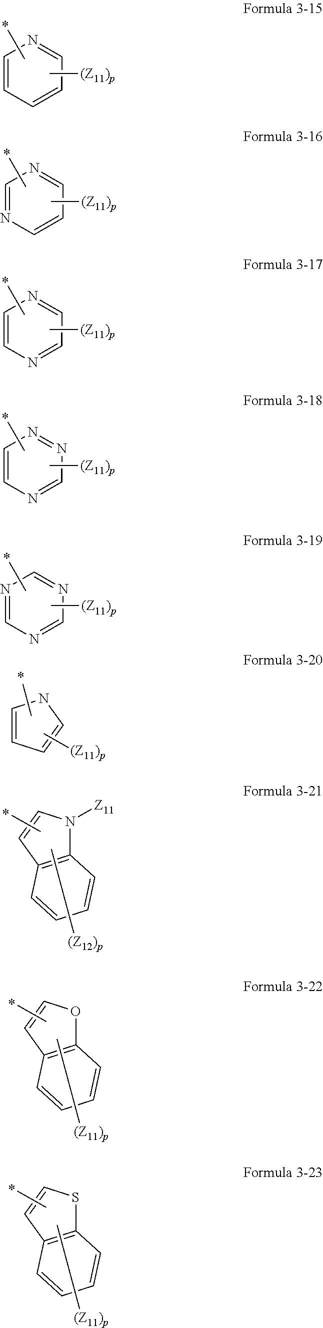 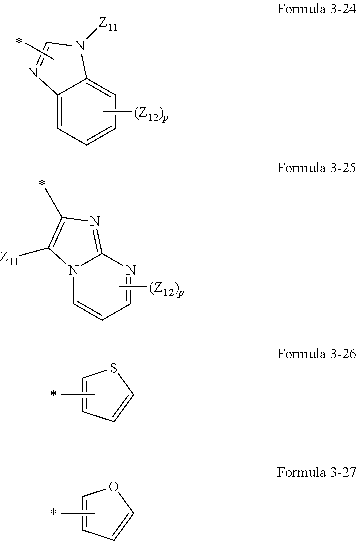
wherein Z₁₁ through Z₁₄ are each independently one of hydrogen; deuterium; a halogen atom; a hydroxyl group; a cyano group; a nitro group; an amino group; an amidino group; hydrazine; hydrazone; a carboxyl group or a salt thereof; a sulfonic acid group or a salt thereof; a phosphoric acid or a salt thereof; a C₁-C₁₀ alkyl group; a C₁-C₁₀ alkoxy group; a C₁-C₁₀ alkyl group and a C₁-C₁₀ alkoxy group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof; a phenyl group; a naphthyl group; a fluorenyl group; a phenanthrenyl group; an anthryl group; a pyrenyl group; a chrysenyl group; a phenyl group, a naphthyl group, a fluorenyl group, a phenanthrenyl group, an anthryl group, a pyrenyl group, and a chrysenyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, and a C₁-C₁₀ alkoxy group; an indolyl group; a benzoimidazolyl group; a carbazolyl group; an imidazolyl group; an imidazolinyl group; an imidazopyridinyl group; an imidazopyrimidinyl group; a pyridinyl group; a pyrimidinyl group; a triazinyl group; a quinolinyl group; and an indolyl group, a benzoimidazolyl group, a carbazolyl group, an imidazolyl group, an imidazolinyl group, an imidazopyridinyl group, an imidazopyrimidinyl group, a pyridinyl group, a pyrimidinyl group, a triazinyl group, and a quinolinyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a phenyl group, and a naphthyl group;
p is an integer of 1 to 9; and
q is an integer of 1 to 4.

12. The condensed-cyclic compound of claim 1, wherein a and b are each independently 1 or 2.

13. The condensed-cyclic compound of claim 1, wherein the condensed-cyclic compound is one of Compounds 1 through 68 below:

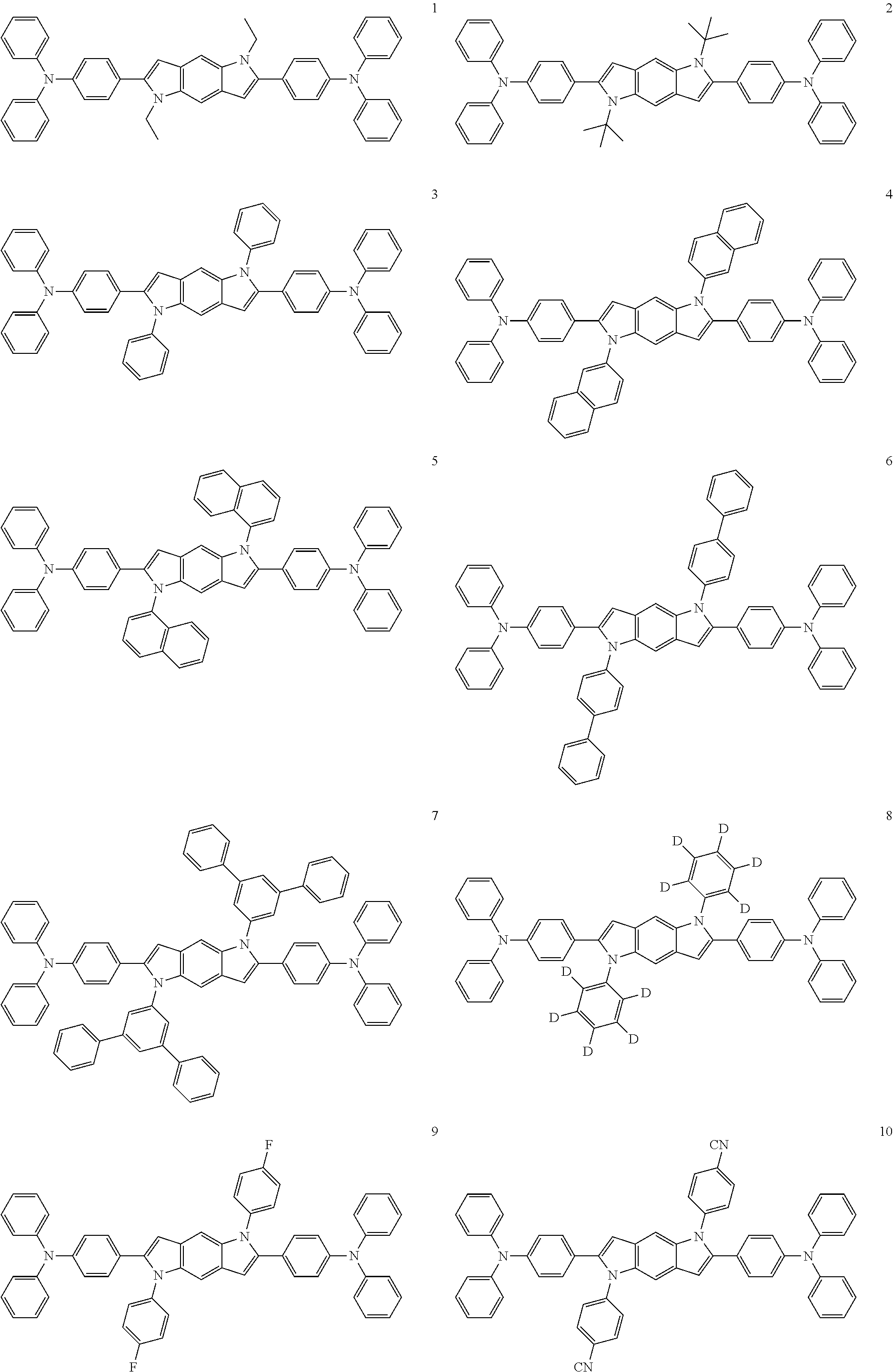

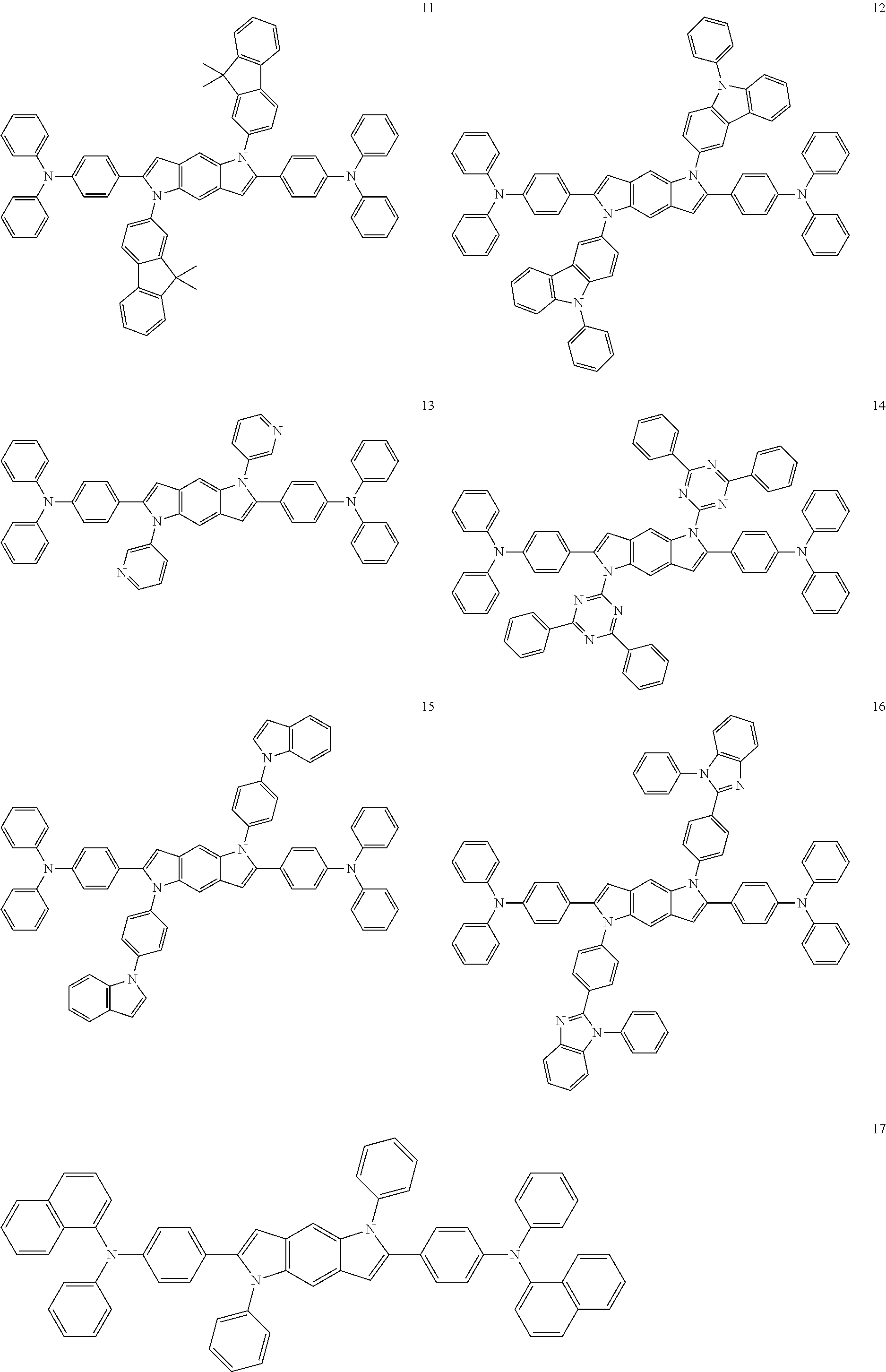

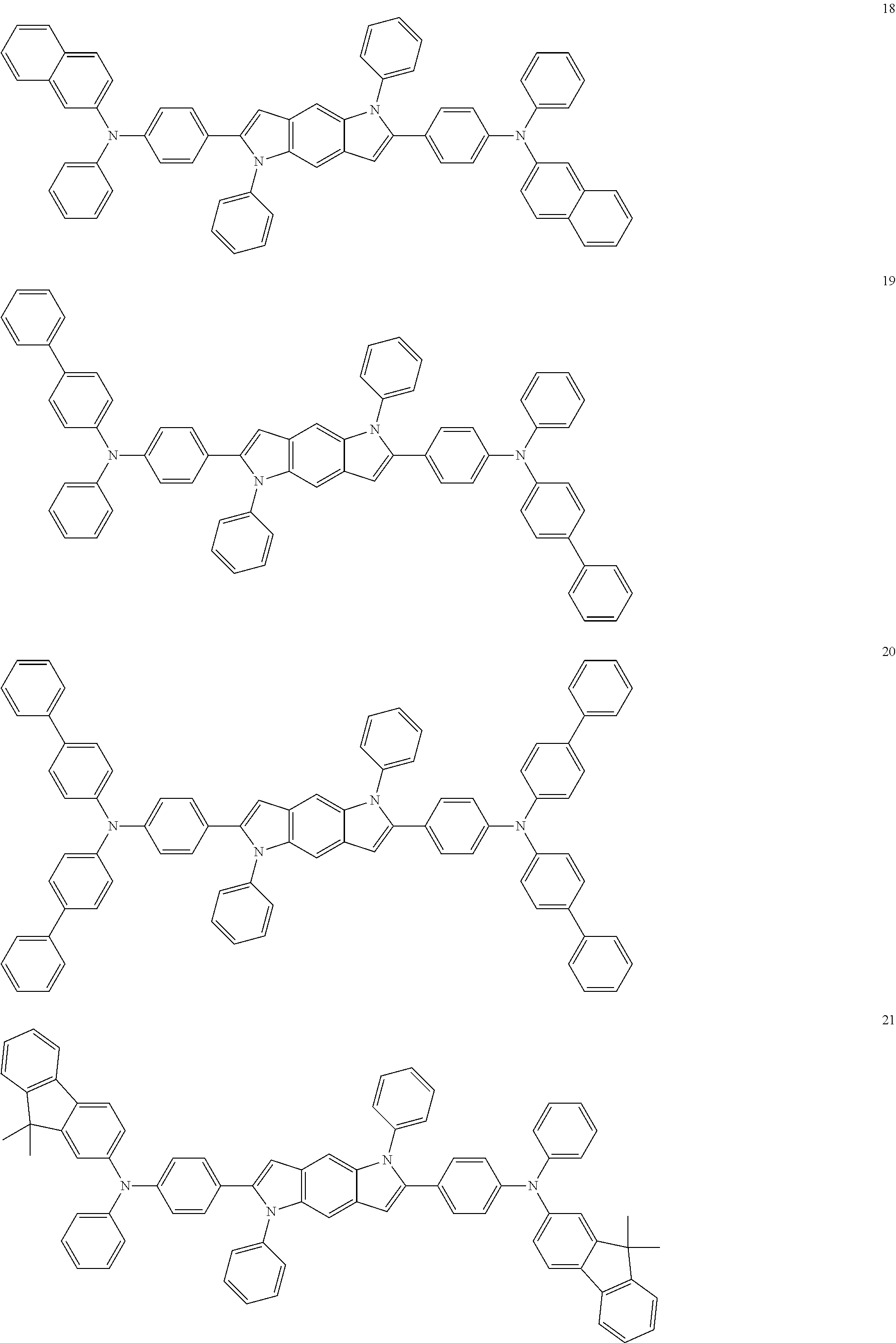

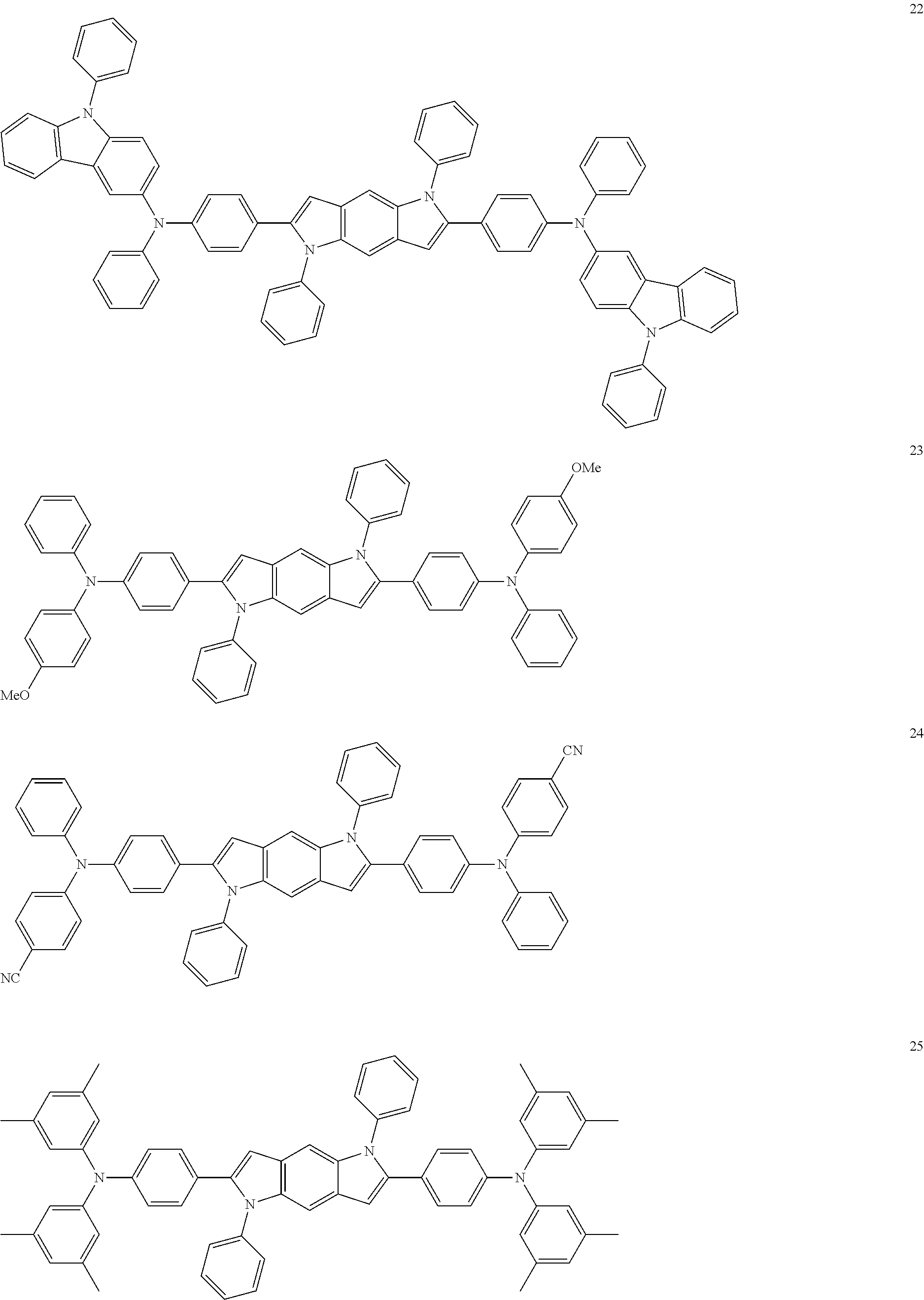

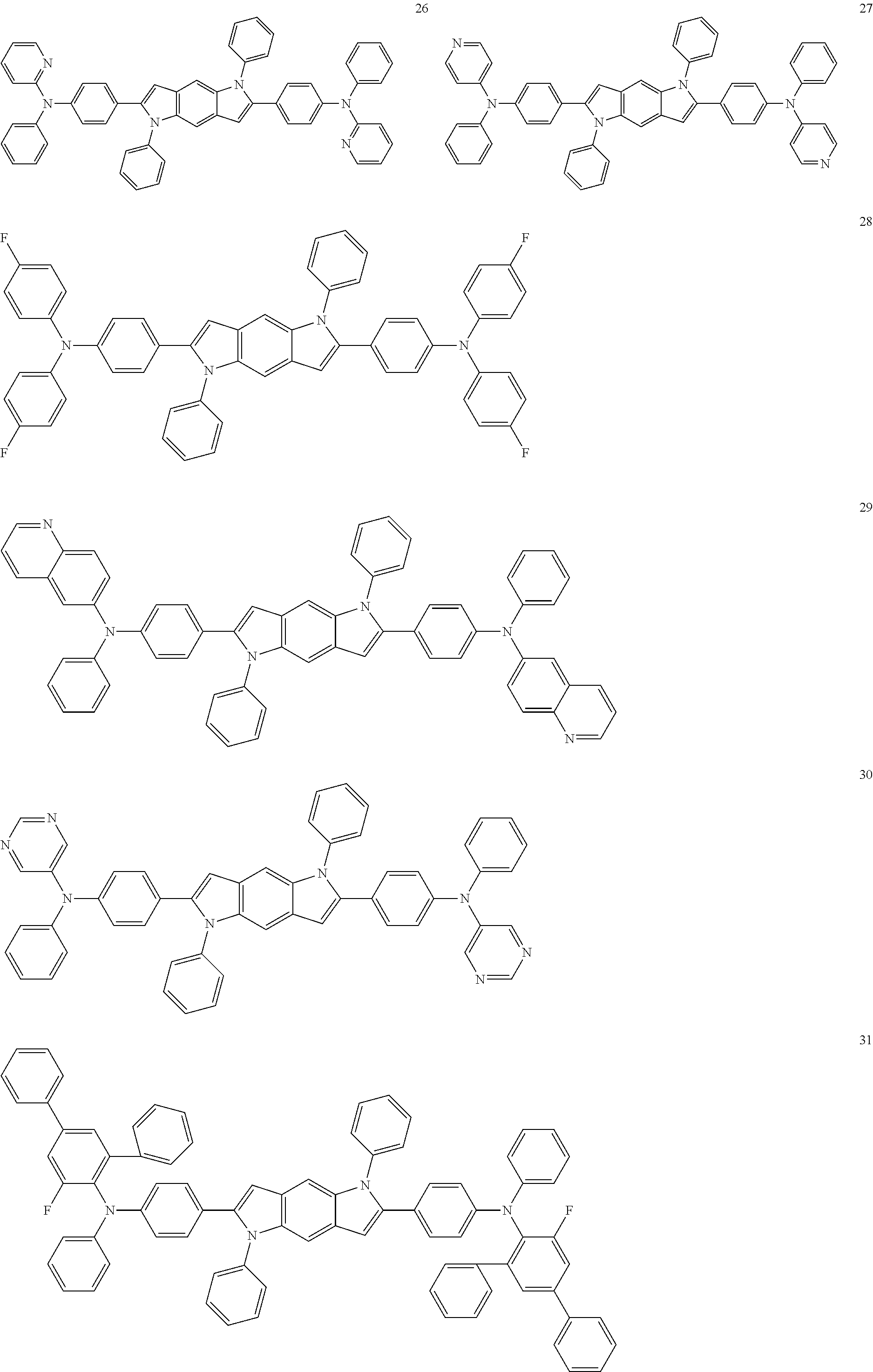

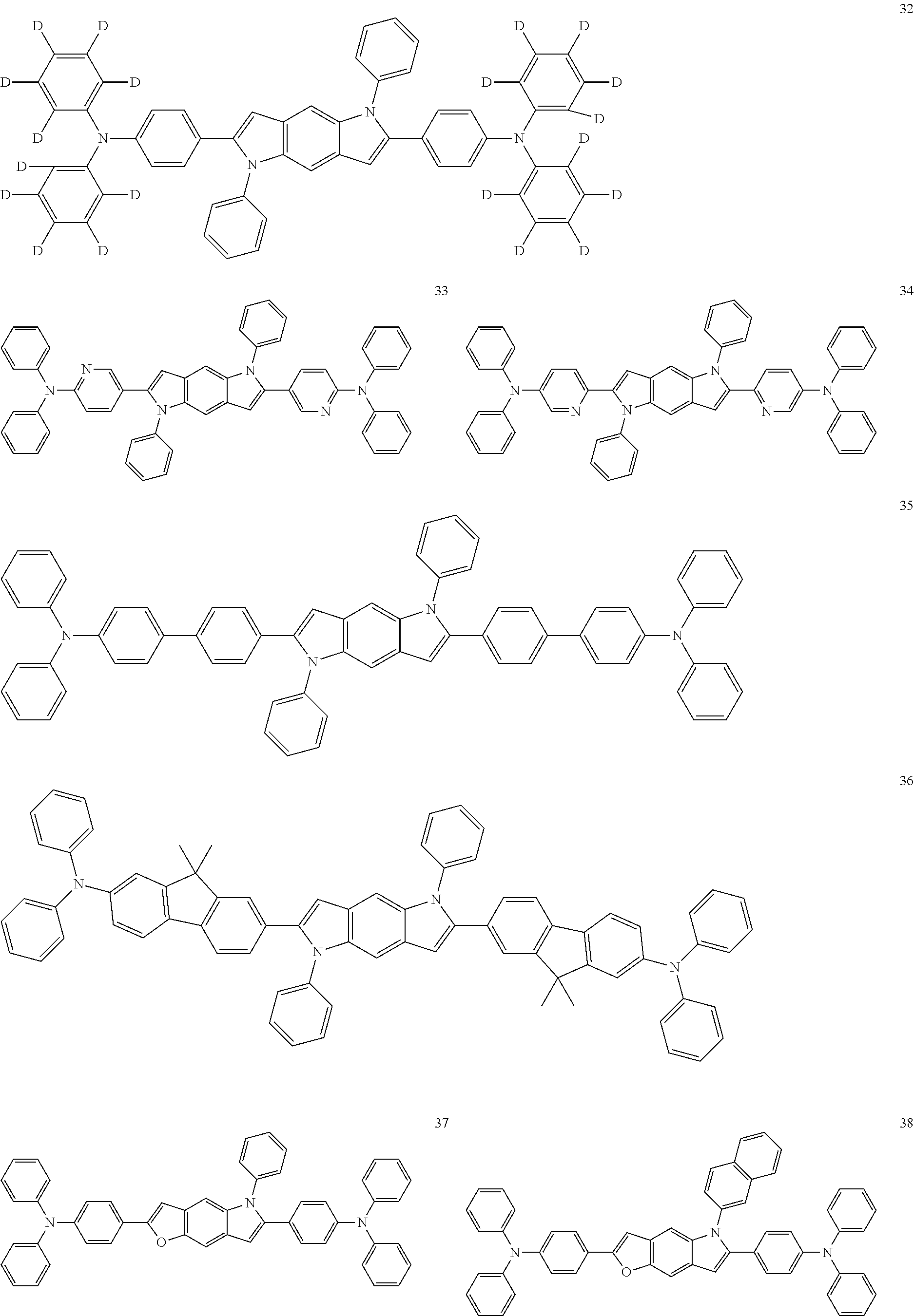

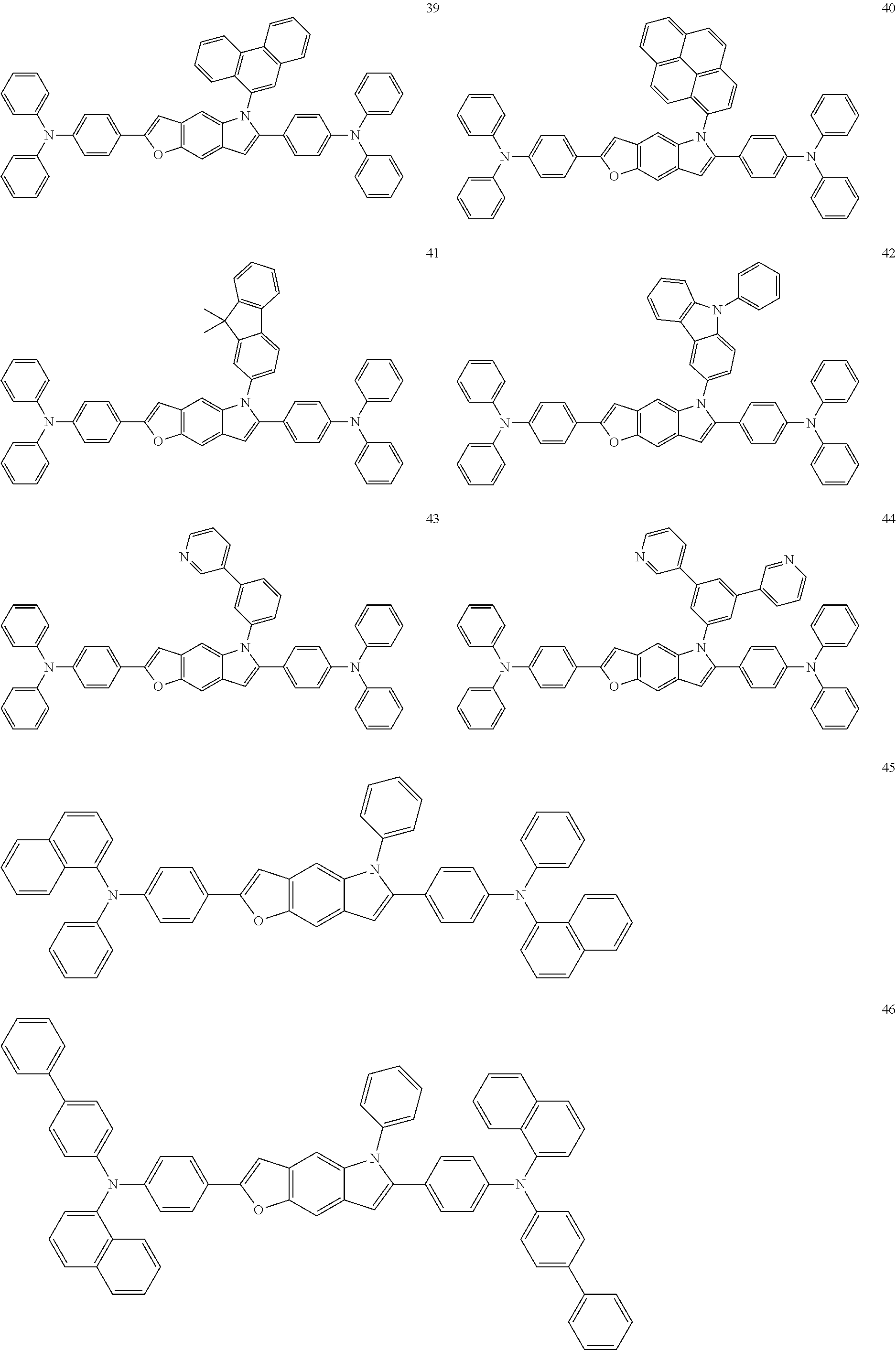

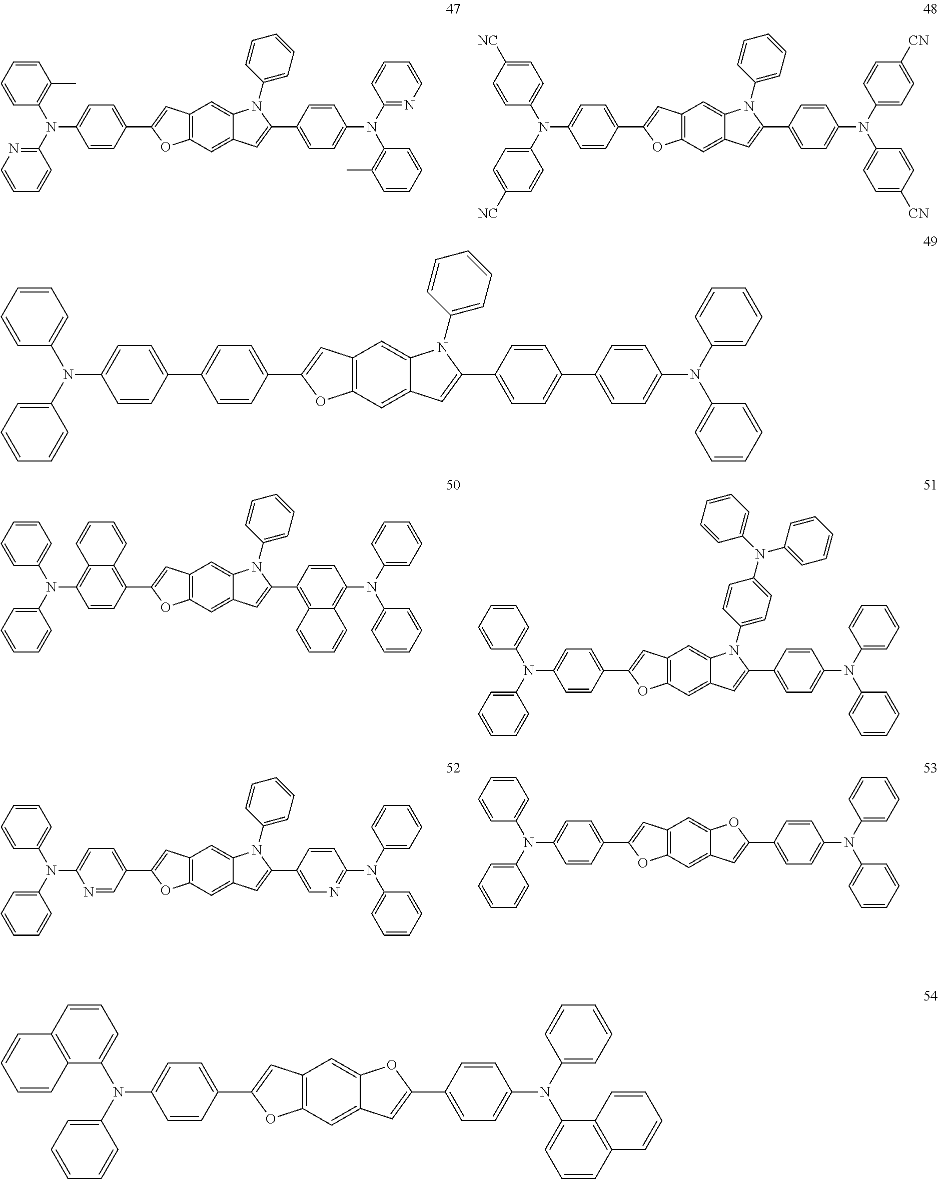

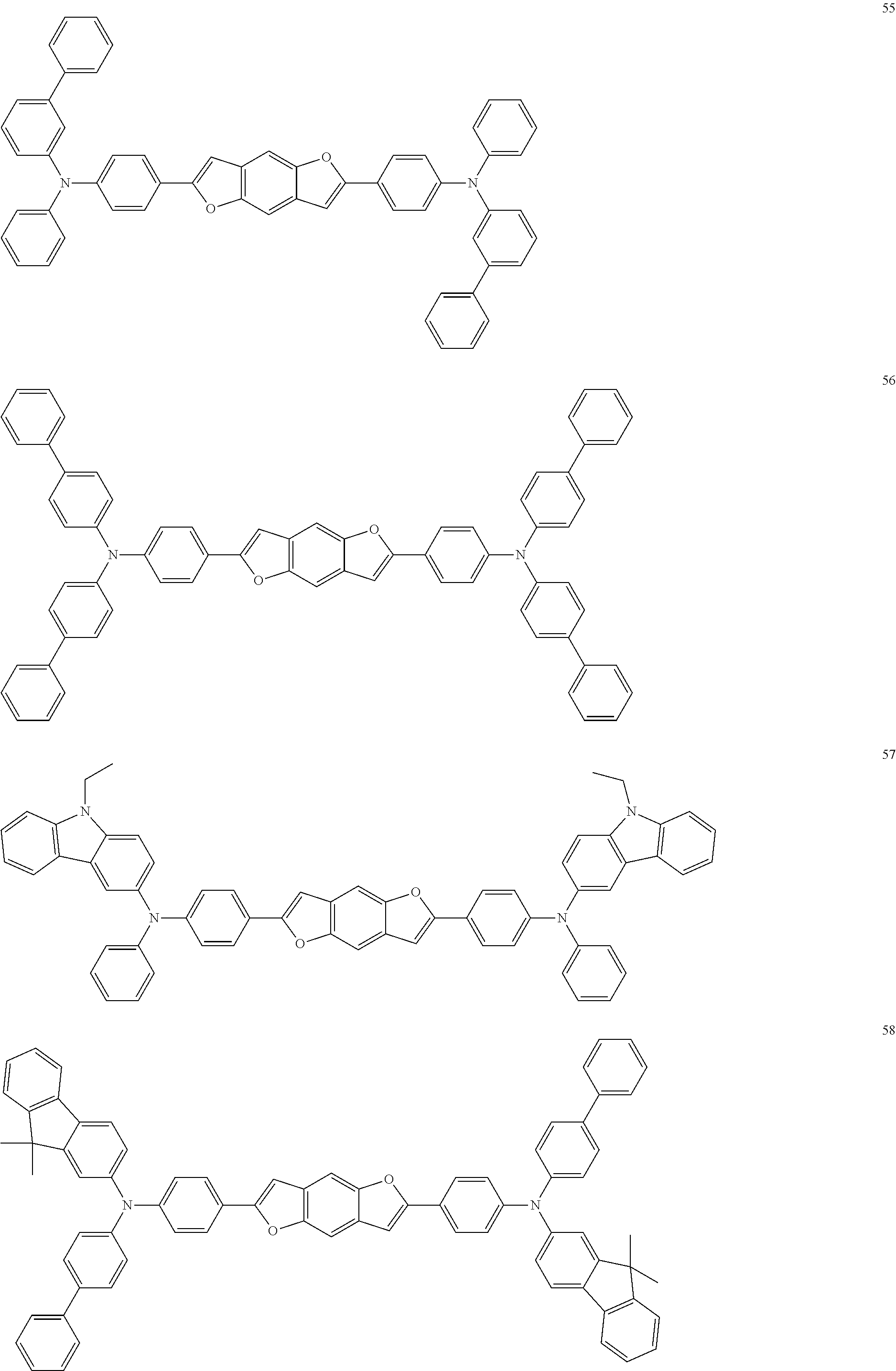

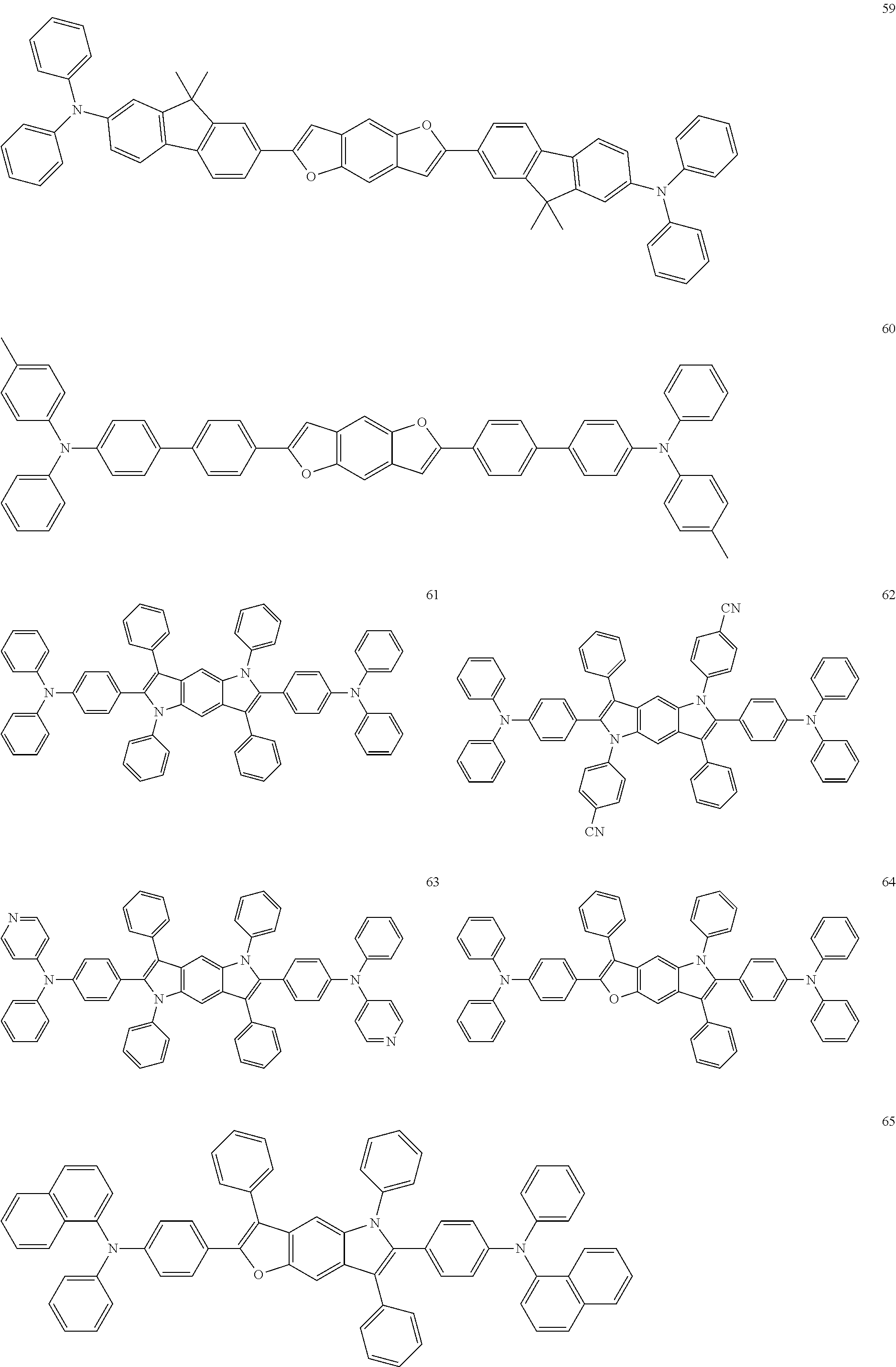

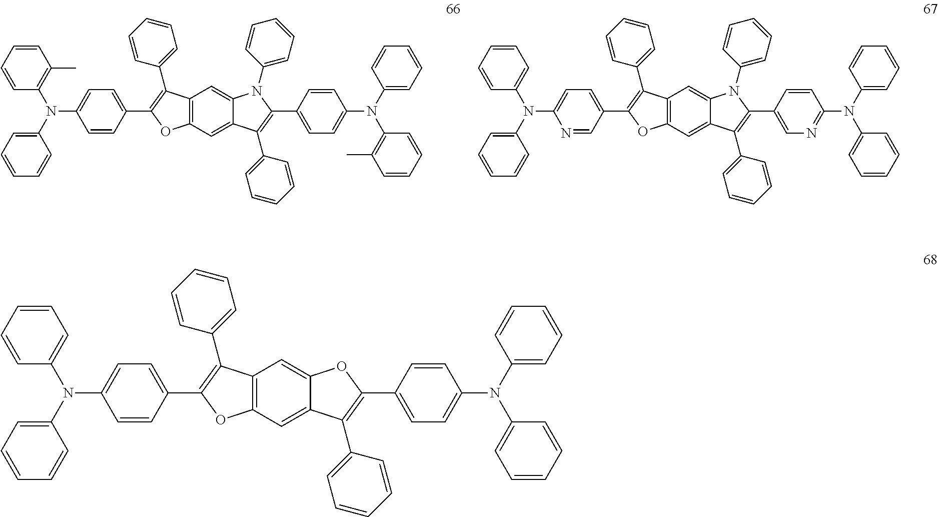 [SMILES: Cc1ccccc1N(c1ccccc1)c1ccc(-c2oc3cc4c(-c5ccccc5)c(-c5ccc(N(c6ccccc6)c6ccccc6C)cc5)n(-c5ccccc5)c4cc3c2-c2ccccc2)cc1 ; c1ccc(-c2c(-c3ccc(N(c4ccccc4)c4ccccc4)nc3)oc3cc4c(-c5ccccc5)c(-c5ccc(N(c6ccccc6)c6ccccc6)nc5)n(-c5ccccc5)c4cc23)cc1 ; c1ccc(-c2c(-c3ccc(N(c4ccccc4)c4ccccc4)cc3)oc3cc4c(-c5ccccc5)c(-c5ccc(N(c6ccccc6)c6ccccc6)cc5)oc4cc23)cc1]

14. An organic light-emitting device comprising
a first electrode;
a second electrode facing the first electrode; and
an organic layer interposed between the first electrode and the second electrode, wherein the organic layer comprises at least one of the condensed-cyclic compounds according to claim 1.

15. The organic light-emitting device of claim 14, wherein the organic layer comprises at least one of a hole injection layer, a hole transport layer, a functional layer having hole injection and hole transport abilities, a buffer layer, an electron blocking layer, an emission layer, a hole blocking layer, an electron transport layer, an electron injection layer, and a functional layer having electron injection and electron transport abilities.

16. The organic light-emitting device of claim 15, wherein the organic layer comprises at least one of a hole injection layer, a hole transport layer, and a functional layer having hole injection and hole transport abilities, wherein at least one of the hole injection layer, the hole transport layer, and the functional layer having hole injection and hole transport abilities comprises the condensed-cyclic compound.

17. The organic light-emitting device of claim 15, wherein the organic layer comprises an emission layer, wherein the emission layer comprises the condensed-cyclic compound.

18. The organic light-emitting device of claim 17, wherein the condensed-cyclic compound in the emission layer acts as a fluorescent dopant.

19. The organic light-emitting device of claim 18, wherein the emission layer further comprises at least one of an anthracene-based compound represented by Formula 400 below and an anthracene-based compound represented by Formula 401 below as a host: 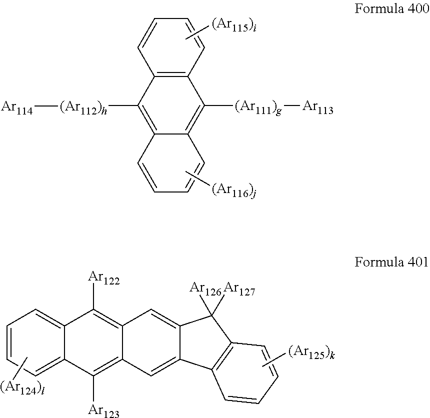
wherein Ar₁₁₁ and Ar₁₁₂ are each independently a substituted or unsubstituted C₅-C₆₀ arylene group;
A₁₁₃ through A₁₁₆ and Ar₁₂₂ through Ar₁₂₅ are each independently a substituted or unsubstituted C₁-C₁₀ alkyl group or a substituted or unsubstituted C₅-C₆₀ aryl group;
Ar₁₂₆ and Ar₁₂₇ are each independently C₁-C₁₀ alkyl group; and
g, h, i, j, k, and l are each independently an integer of 0 to 4.

20. The organic light-emitting device of claim 15, wherein the organic layer comprises at least one of a hole injection layer, a hole transport layer, and a functional layer having hole injection and hole transport abilities and an emission layer, wherein the at least one of a hole injection layer, a hole transport layer, and a functional layer having hole injection and hole transport abilities and an emission layer comprises the condensed-cyclic compound, wherein the condensed-cyclic compound in the at least one of a hole injection layer, a hole transport layer, and a functional layer having hole injection and hole transport abilities is different from the condensed-cyclic compound in the emission layer.

21. The organic light-emitting device of claim 20, wherein the condensed-cyclic compound in the emission layer acts as a fluorescent dopant.

22. The organic light-emitting device of claim 15, wherein the organic layer comprises an electron transport layer, wherein the electron transport layer comprises an electron transporting organic compound and a metal complex.
